(19) United States
(12) Patent Application Publication
(10) Pub. No.: US 2021/0369759 A1
(43) Pub. Date: Dec. 2, 2021

(54) METHODS AND COMPOSITIONS FOR NUCLEIC ACID EXPRESSION INVOLVING INHIBITION of NF-kB PATHWAYS AND/OR IRF PATHWAYS

(71) Applicant: Helix Nanotechnologies, Inc., Walnut, CA (US)

(72) Inventors: Nikolai Eroshenko, Boston, MA (US); Hannu Rajaniemi, San Francisco, CA (US); Nikhil Dhar, Boston, MA (US)

(21) Appl. No.: 17/163,168

(22) Filed: Jan. 29, 2021

Related U.S. Application Data
(60) Provisional application No. 62/967,341, filed on Jan. 29, 2020.

(51) Int. Cl. A61K 31/7105; C07K 14/47

(52) U.S. Cl. CPC A61K 31/7105; C07K 14/4703

(57) ABSTRACT

Nucleic acid expression systems for delivery of oligonucleotides, such as RNA oligonucleotides, to target cells and methods of using the same are provided herein. Exemplary nucleic acid expression systems for expression of a payload sequence include at least one composition that delivers an inhibitor of a NF-κB pathway and/or an inhibitor of an IRF pathway. Provided herein are also pharmaceutical compositions that deliver an inhibitor of a NF-κB pathway and/or an inhibitor of an IRF pathway, and methods of using the same for enhancing expression of an oligonucleotide, such as an RNA oligonucleotide.

### CROSS-REFERENCE TO RELATED APPLICATIONS

This application claims the benefit of U.S. Provisional Application No. 62/967,341 filed Jan. 29, 2020, the contents of which are hereby incorporated herein in their entirety.

### SEQUENCE LISTING

The instant application contains a Sequence Listing which has been submitted electronically in ASCII format and is hereby incorporated by reference in its entirety. Said ASCII copy, created on May 21, 2021s, is named 2012611-0024_SL.txt and is 10,335 bytes in size.

### BACKGROUND

In recent years, research progress has been made to gene therapy or RNA therapy for treating or improving a particular condition or disease. In particular, mRNA vectors have emerged as a robust therapeutic modality that can be used for gene therapy, vaccines, immuno-oncology, and other applications that involve high levels of transgene expression. However, mRNA-based therapies currently have significant technological limitations.

### SUMMARY

The present disclosure provides technologies for delivering oligonucleotides, e.g., in some embodiments, RNA oligonucleotides, to a subject or a target cell. The present disclosure, among other things, recognizes that at least one challenge associated with oligonucleotide delivery includes developing RNA therapies that do not activate the myriad innate immune sensors while still efficiently recognized by a translational machinery in a cell. For example, in vitro-synthesized mRNA molecules generally contain long dsRNA, uncapped products, and other impurities that can be detected by innate immune sensors, e.g., TLR3, TLR7, TLR8, MDA5, RIG-I, PKR, OAS, and/or other intracellular sensors.

Among other things, the present disclosure identifies the source of a problem with certain prior technologies including, for example, certain conventional approaches to reduce immunogenicity of synthetic RNA. For example, the present disclosure appreciates that many conventional approaches, e.g., based on chemically modified nucleotides and viral inhibitors of RNA sensors, to reduce the ability of individual innate immune sensors can be insufficient or inefficient to suppress innate immune response upon oligonucleotide delivery. In some embodiments, the present disclosure provides technologies (including systems, compositions, and methods) that address such problems, among other things, by targeting the signaling “bottleneck” between certain innate immune sensors and downstream amplification cascades.

Among other things, the present disclosure encompasses a recognition that signaling through certain innate immune sensors (e.g., TLR3, TLR7, TLR8, MDA5, and/or RIG-I) converges on two signaling pathways: Nuclear Factor kappa-light chain enhancer of activated B cells (NF-κB) and Interferon Regulatory Factors (IRF), which in many embodiments, can jointly induce an innate immune response and/or an anti-viral state in a target cell. The present disclosure, among other things, also recognizes that activation of NF-κB and/or IRF signaling (e.g., high levels of NF-κB and IRF signaling) can lead to induction of an innate immune response and/or an anti-viral state in the surrounding cells and/or tissue and/or reduce expression of an introduced nucleic acid molecule (e.g., an RNA oligonucleotide, such as a mRNA oligonucleotide).

The present disclosure provides, among other things, a unique approach for reducing the immunogenicity of synthetic nucleic acid molecules (e.g., synthetic RNA oligonucleotides) by targeting one or more signaling components downstream of certain innate immune sensors rather than the innate immune sensors themselves or secreted proteins associated with an innate immune response and/or an anti-viral state. In some embodiments, the present disclosure provides an approach for reducing the immunogenicity of a synthetic nucleic acid molecule (e.g., synthetic RNA oligonucleotide) by co-delivering a synthetic nucleic acid molecule (e.g., synthetic RNA oligonucleotide) with one or more agents that inhibit a NF-κB pathway and/or an IRF pathway. In some embodiments, the present disclosure provides nucleic acid expression systems and compositions for delivery of a nucleic acid (e.g., RNA oligonucleotide, e.g., mRNA) comprising a payload sequence with a composition that inhibits a NF-κB pathway and/or an IRF pathway.

In some embodiments, nucleic acid expression systems, compositions, and/or methods described herein may be useful for increasing expression of a payload oligonucleotide (e.g., RNA oligonucleotide, e.g., mRNA) and/or reducing innate immunity-trigger suppression of protein translation and/or reducing degradation of introduced payload oligonucleotide (e.g., RNA oligonucleotide, e.g., mRNA) in target cells. In some embodiments, nucleic acid expression systems, compositions, and/or methods described herein may be useful for enhancing viability of a target cell upon delivery of a payload oligonucleotide (e.g., RNA oligonucleotide, e.g., mRNA). In some embodiments, nucleic acid expression systems, compositions, and/or methods described herein may be useful for reducing non-specific toxicity induced in a target cell by an introduced payload oligonucleotide (e.g., RNA oligonucleotide, e.g., mRNA). In some embodiments, nucleic acid expression systems, compositions, and/or methods described herein may be useful in applications or circumstances where repeated dosing of a payload oligonucleotide (e.g., RNA oligonucleotide, e.g., mRNA) is desirable.

In one aspect, provided herein is a nucleic acid expression system comprising: (i) an oligonucleotide comprising a payload sequence, and (ii) at least one composition that delivers an inhibitor of a NF-κB pathway and/or an inhibitor of an IRF pathway. In some embodiments, an oligonucleotide comprising a payload sequence is an RNA oligonucleotide (e.g., mRNA oligonucleotide) comprising a payload sequence.

In some embodiments, an oligonucleotide (e.g., RNA oligonucleotide) comprising a payload sequence provided in a nucleic acid expression system is delivered in a separate composition from one or more compositions that deliver an inhibitor of a NF-κB pathway and/or an inhibitor of an IRF pathway. Accordingly, in some embodiments, a nucleic acid expression system comprises (i) a composition that includes an oligonucleotide comprising a payload sequence; and (ii) one or more compositions that deliver an inhibitor of a NF-κB pathway and/or an inhibitor of an IRF pathway.

In some embodiments, a nucleic acid expression system comprises: (i) an oligonucleotide (e.g., an RNA oligonucleotide such as, e.g., mRNA) comprising a payload sequence, and (ii) a composition that delivers an inhibitor of a NF-κB pathway. For example, in some such embodiments, a composition that delivers an inhibitor of a NF-κB pathway includes a polypeptide inhibitor of a NF-κB pathway or a nucleic acid (e.g., an RNA oligonucleotide) comprising a sequence that encodes an inhibitor of a NF-κB pathway. In some embodiments, a composition that delivers an inhibitor of a NF-κB pathway includes an RNA oligonucleotide comprising a sequence that encodes an inhibitor of a NF-κB pathway.

In some embodiments, a nucleic acid expression system comprises: (i) an oligonucleotide (e.g., an RNA oligonucleotide such as, e.g., mRNA) comprising a payload sequence, and (ii) a composition that delivers an inhibitor of an IRF pathway. For example, in some such embodiments, a composition that delivers an inhibitor of an IRF pathway includes a polypeptide inhibitor of an IRF pathway or a nucleic acid (e.g., an RNA oligonucleotide) comprising a sequence that encodes an inhibitor of an IRF pathway. In some embodiments, a composition that delivers an inhibitor of an IRF pathway includes an RNA oligonucleotide comprising a sequence that encodes an inhibitor of an IRF pathway.

In some embodiments, a nucleic acid expression system comprises: (i) an oligonucleotide (e.g., an RNA oligonucleotide such as, e.g., mRNA) comprising a payload sequence, and (ii) one or more compositions that deliver an inhibitor of a NF-κB pathway and an inhibitor of an IRF pathway. For example, in some such embodiments, one or more compositions that deliver an inhibitor of a NF-κB pathway and an inhibitor of an IRF pathway include a polypeptide inhibitor of a NF-κB pathway and a polypeptide inhibitor of an IRF pathway. In some embodiments, one or more compositions that deliver an inhibitor of a NF-κB pathway and an inhibitor of an IRF pathway include a polypeptide inhibitor of a NF-κB pathway and a nucleic acid (e.g., an RNA oligonucleotide) comprising a sequence that encodes an inhibitor of an IRF pathway. In some embodiments, one or more compositions that deliver an inhibitor of a NF-κB pathway and an inhibitor of an IRF pathway include a nucleic acid (e.g., an RNA oligonucleotide) comprising a sequence that encodes an inhibitor of a NF-κB pathway and a polypeptide inhibitor of an IRF pathway. In some embodiments, one or more compositions that deliver an inhibitor of a NF-κB pathway and an inhibitor of an IRF pathway include a nucleic acid (e.g., an RNA oligonucleotide) comprising a sequence that encodes an inhibitor of a NF-κB pathway and a nucleic acid (e.g., an RNA oligonucleotide) comprising a sequence that encodes an inhibitor of an IRF pathway. In some embodiments, one or more compositions that deliver an inhibitor of a NF-κB pathway and an inhibitor of an IRF pathway include an RNA oligonucleotide comprising a sequence that encodes an inhibitor of a NF-κB pathway and an RNA oligonucleotide comprising a sequence that encodes an inhibitor of an IRF pathway.

In some embodiments where a nucleic acid expression system comprises one or more compositions that deliver an inhibitor of a NF-κB pathway and an inhibitor of an IRF pathway, such inhibitors of a NF-κB pathway and of an IRF pathway may be delivered in separate compositions or in the same composition. For example, in some embodiments, an inhibitor of a NF-κB pathway and an inhibitor of an IRF pathway may be delivered in the same composition. In some embodiments, such a single composition comprising both inhibitors of a NF-κB pathway and of an IRF pathway may comprise (i) an RNA oligonucleotide comprising a sequence that encodes an inhibitor of a NF-κB pathway and (ii) an RNA oligonucleotide comprising a sequence that encodes an inhibitor of an IRF pathway. In some such embodiments, a sequence encoding an inhibitor of a NF-κB pathway and a sequence encoding an inhibitor of an IRF pathway may be present in the same RNA oligonucleotide. In some such embodiments, a sequence encoding an inhibitor of a NF-κB pathway and a sequence encoding an inhibitor of an IRF pathway may be present in different RNA oligonucleotides.

In some embodiments, an oligonucleotide comprising a payload sequence is an RNA oligonucleotide. In some embodiments, an oligonucleotide comprising a payload sequence is a synthetic RNA oligonucleotide. In some embodiments, an oligonucleotide comprising a payload sequence is a messenger (mRNA) oligonucleotide (e.g., a synthetic mRNA oligonucleotide). In some embodiments, such a synthetic RNA or mRNA oligonucleotide may be produced by in vitro transcription.

In some embodiments, an oligonucleotide comprising a sequence that encodes an inhibitor of a NF-κB pathway is an RNA oligonucleotide. In some embodiments, an oligonucleotide comprising a sequence that encodes an inhibitor of a NF-κB pathway is a synthetic RNA oligonucleotide. In some embodiments, an oligonucleotide comprising a sequence that encodes an inhibitor of a NF-κB pathway is a mRNA oligonucleotide (e.g., a synthetic mRNA oligonucleotide). In some embodiments, such a synthetic RNA or mRNA oligonucleotide may be produced by in vitro transcription.

In some embodiments, an oligonucleotide comprising a sequence that encodes an inhibitor of an IRF pathway is an RNA oligonucleotide. In some embodiments, an oligonucleotide comprising a sequence that encodes an inhibitor of an IRF pathway is a synthetic RNA oligonucleotide. In some embodiments, an oligonucleotide comprising a sequence that encodes an inhibitor of an IRF pathway is a mRNA oligonucleotide (e.g., a synthetic mRNA oligonucleotide). In some embodiments, such a synthetic RNA or mRNA oligonucleotide may be produced by in vitro transcription.

In some embodiments involving a composition that delivers an inhibitor of a NF-κB pathway, such an inhibitor of NF-κB pathway may be or comprise a viral polypeptide, which in some embodiments, may be delivered as a viral polypeptide inhibitor, or in some embodiments, may be delivered as a nucleic acid (e.g., an RNA oligonucleotide) comprising a sequence that encodes such a viral polypeptide inhibitor. In some embodiments, an inhibitor of a NF-κB pathway to be delivered is or comprises an agent (e.g., a polypeptide-based or nucleic acid-based agent) that inhibits activity and/or formation of IκB kinase (IKK) complex. For example, in some embodiments, such an agent that inhibits activity and/or formation of IKK complex is or comprises an agent (e.g., a polypeptide-based or nucleic acid-based agent) that binds to and/or inhibits activity and/or interaction of at least one of an IKKα subunit, an IKKβ subunit, and an IKKγ subunit. In some embodiments, an agent that binds to and/or inhibits activity and/or interaction of an IKKβ subunit is or comprises a Vaccinia Virus Protein B14 polypeptide (e.g., a wild-type B14 polypeptide) and/or a nucleic acid (e.g., an RNA oligonucleotide) comprising a sequence that encoding the same. In some embodiments, a Vaccinia Virus Protein B14 polypeptide includes an amino acid sequence that is at least about 80%, 85%, 90%, 95%, 96%, 97%, 98%, 99% or higher (including and up to 100%) identical to a wild-type Vaccinia Virus Protein B14. In some embodiments, an exemplary wild-type Vaccinia Virus protein B14 has the amino acid sequence of SEQ ID NO: 5. In some embodiments, an inhibitor of NF-κB signaling is or comprises a B14 polypeptide that comprises the amino acid sequence of SEQ ID NO: 5 or a nucleic acid (e.g., an RNA oligonucleotide) encoding such a B14 polypeptide.

In some embodiments involving a composition that delivers an inhibitor of an IRF pathway, such an inhibitor of an IRF pathway may be or comprise a viral polypeptide, which in some embodiments, may be delivered as a viral polypeptide inhibitor, or in some embodiments, may be delivered as a nucleic acid (e.g., an RNA oligonucleotide) comprising a sequence that encodes such a viral polypeptide inhibitor. In some embodiments, an inhibitor of an IRF pathway to be delivered is or comprises an agent (e.g., a polypeptide-based or nucleic acid-based agent) that inhibits activity and/or formation of a complex comprising TANK-binding kinase 1 (TBK1) and IκB kinase ε (IKKε). For example, in some embodiments, such an agent that inhibits activity and/or formation of the TBK1/IKKε complex is or comprises an agent (e.g., a polypeptide-based or nucleic acid-based agent) that binds to and/or inhibits activity and/or interaction of DEAD box protein 3 (DDX3) with the TBK1/IKKε complex, which, for example, in some embodiments, may be or comprise a Vaccinia Virus Protein K7 polypeptide (e.g., a wild-type K7 polypeptide) and/or a nucleic acid (e.g., an RNA oligonucleotide) comprising a sequence that encoding the same. In some embodiments, a Vaccinia Virus Protein K7 polypeptide includes an amino acid sequence that is at least about 80%, 85%, 90%, 95%, 96%, 97%, 98%, 99% or higher (including and up to 100%) identical to a wild-type Vaccinia Virus Protein K7. In some embodiments, an exemplary wild-type Vaccinia Virus Protein K7 has the amino acid sequence of SEQ ID NO: 9. In some embodiments, an inhibitor of IRF signaling is or comprises a K7 polypeptide that comprises the amino acid sequence of SEQ ID NO: 9 and/or a nucleic acid (e.g., an RNA oligonucleotide) encoding such a K7 polypeptide.

In some embodiments involving a composition that delivers an inhibitor of an IRF pathway, such an inhibitor of an IRF pathway is or comprises an inhibitor of a JAK-STAT pathway, which in some embodiments, may be delivered as a polypeptide inhibitor, or in some embodiments, may be delivered as a nucleic acid (e.g., an RNA oligonucleotide) comprising a sequence that encodes such a polypeptide inhibitor. In some embodiments, an inhibitor of a JAK-STAT pathway is or comprises an agent (e.g., a polypeptide-based or nucleic acid-based agent) that inhibits activity and/or formation of a complex comprising STAT1, STAT2, and IRF9. For example, in some embodiments, an agent that inhibits activity and/or formation of a STAT1/STAT2/IRF9 complex is or comprises an agent (e.g., a polypeptide-based or nucleic acid-based agent) that binds to and/or inhibits activity and/or interaction of at least one of STAT1, STAT2, and IRF9. In some embodiments, such an agent that inhibits activity and/or formation of a STAT1/STAT2/IRF9 complex is or comprises an agent (e.g., a polypeptide-based or nucleic acid-based agent) that binds to and/or inhibits activity of STAT2. In some embodiments, such an agent that binds to and/or inhibits activity of STAT2 is or comprises a Vaccinia Virus Protein C6 polypeptide (e.g., a wild-type C6 polypeptide) and/or a nucleic acid (e.g., an RNA oligonucleotide) comprising a sequence that encoding the same. In some embodiments, a Vaccinia Virus Protein C6 polypeptide includes an amino acid sequence that is at least about 80%, 85%, 90%, 95%, 96%, 97%, 98%, 99% or higher (including and up to 100%) identical to a wild-type Vaccinia Virus Protein C6. In some embodiments, an exemplary wild-type Vaccinia Virus protein C6 has the amino acid sequence of SEQ ID NO: 12. In some embodiments, an inhibitor of IRF signaling is or comprises a C6 polypeptide that comprises the amino acid sequence of SEQ ID NO: 12 and/or a nucleic acid (e.g., an RNA oligonucleotide) encoding such a C6 polypeptide.

In some embodiments involving one or more compositions that delivers at least one inhibitor of a NF-κB pathway and at least one inhibitor of an IRF pathway, such compositions may be formulated to comprise one or more compositions that deliver a Vaccinia Virus Protein B14 polypeptide and Vaccinia Virus Protein K7 polypeptide. In some embodiments, such compositions may be formulated to comprise one or more compositions that deliver a Vaccinia Virus Protein B14 polypeptide and Vaccinia Virus Protein C6 polypeptide. In some embodiments, such compositions may be formulated to comprise one or more compositions that deliver a Vaccinia Virus Protein K7 polypeptide and Vaccinia Virus Protein C6 polypeptide. In some embodiments, such compositions may be formulated to comprise one or more compositions that deliver a Vaccinia Virus Protein B14 polypeptide, a Vaccinia Virus Protein K7 polypeptide, and a Vaccinia Virus Protein C6 polypeptide.

Another aspect of the present disclosure provides compositions comprising any embodiments of nucleic acid expression systems as described herein, or one or more components thereof. In some embodiments, a composition comprising a nucleic acid expression system (e.g., ones described herein) or one or more components thereof is a pharmaceutical composition. In some embodiments, such a pharmaceutical composition may further comprise a pharmaceutically acceptable carrier.

Cells comprising a nucleic acid expression system (e.g., ones as described herein), or one or more components thereof are also within the scope of the present disclosure. In some embodiments, such a cell is an eukaryotic cell, which, for example, in some embodiments may be a mammalian cell.

In some embodiments, the present disclosure provides pharmaceutical compositions that deliver one or more inhibitors of a NF-κB pathway (e.g., ones as described herein) and/or one or more inhibitors of an IRF pathway (e.g., ones as described herein). In some embodiments, the present disclosure provides pharmaceutical compositions that deliver one or more inhibitors of a NF-κB pathway (e.g., ones as described herein) and one or more inhibitors of an IRF pathway (e.g., ones as described herein).

In some embodiments, a provided pharmaceutical composition that delivers one or more inhibitors of a NF-κB pathway and one or more inhibitors of an IRF pathway may comprise a polypeptide inhibitor of a NF-κB pathway and a polypeptide inhibitor of an IRF pathway. In some embodiments, a provided pharmaceutical composition that delivers one or more inhibitors of a NF-κB pathway and one or more inhibitors of an IRF pathway may comprise a polypeptide inhibitor of a NF-κB pathway and a nucleic acid (e.g., an RNA oligonucleotide) comprising a sequence that encodes an inhibitor of an IRF pathway. In some embodiments, a provided pharmaceutical composition that delivers one or more inhibitors of a NF-κB pathway and one or more inhibitors of an IRF pathway may comprise a nucleic acid (e.g., an RNA oligonucleotide) comprising a sequence that encodes an inhibitor of a NF-κB pathway and a polypeptide inhibitor of an IRF pathway. In some embodiments, a provided pharmaceutical composition that delivers one or more inhibitors of a NF-κB pathway and one or more inhibitors of an IRF pathway may comprise a nucleic acid (e.g., an RNA oligonucleotide) comprising a sequence that encodes an inhibitor of a NF-κB pathway and a nucleic acid (e.g., an RNA oligonucleotide) comprising a sequence that encodes an inhibitor of an IRF pathway. In some embodiments, a provided pharmaceutical composition that delivers one or more inhibitors of a NF-κB pathway and one or more inhibitors of an IRF pathway may comprise an RNA oligonucleotide comprising a sequence that encodes an inhibitor of a NF-κB pathway and an RNA oligonucleotide comprising a sequence that encodes an inhibitor of an IRF pathway.

In some embodiments where a provided pharmaceutical composition comprises one or more compositions that deliver an inhibitor of a NF-κB pathway and an inhibitor of an IRF pathway, such inhibitors of a NF-κB pathway and of an IRF pathway may be delivered in separate compositions or in the same composition. For example, in some embodiments, an inhibitor of a NF-κB pathway and an inhibitor of an IRF pathway may be delivered in the same composition. In some embodiments, such a single composition comprising both inhibitors of a NF-κB pathway and of an IRF pathway may comprise (i) an RNA oligonucleotide comprising a sequence that encodes an inhibitor of a NF-κB pathway and (ii) an RNA oligonucleotide comprising a sequence that encodes an inhibitor of an IRF pathway. In some such embodiments, a sequence encoding an inhibitor of a NF-κB pathway and a sequence encoding an inhibitor of an IRF pathway may be present in the same RNA oligonucleotide. In some such embodiments, a sequence encoding an inhibitor of a NF-κB pathway and a sequence encoding an inhibitor of an IRF pathway may be present in different RNA oligonucleotides.

In some embodiments, an oligonucleotide comprising a sequence that encodes an inhibitor of a NF-κB pathway is an RNA oligonucleotide. In some embodiments, an oligonucleotide comprising a sequence that encodes an inhibitor of a NF-κB pathway is a synthetic RNA oligonucleotide. In some embodiments, an oligonucleotide comprising a sequence that encodes an inhibitor of a NF-κB pathway is a mRNA oligonucleotide (e.g., a synthetic mRNA oligonucleotide). In some embodiments, such a synthetic RNA or mRNA oligonucleotide may be produced by in vitro transcription.

In some embodiments, an oligonucleotide comprising a sequence that encodes an inhibitor of an IRF pathway is an RNA oligonucleotide. In some embodiments, an oligonucleotide comprising a sequence that encodes an inhibitor of an IRF pathway is a synthetic RNA oligonucleotide. In some embodiments, an oligonucleotide comprising a sequence that encodes an inhibitor of an IRF pathway is a mRNA oligonucleotide (e.g., a synthetic mRNA oligonucleotide). In some embodiments, such a synthetic RNA or mRNA oligonucleotide may be produced by in vitro transcription.

In some embodiments involving a pharmaceutical composition that delivers an inhibitor of a NF-κB pathway, such an inhibitor of NF-κB pathway may be or comprise a viral polypeptide, which in some embodiments, may be delivered as a viral polypeptide inhibitor, or in some embodiments, may be delivered as a nucleic acid (e.g., an RNA oligonucleotide) comprising a sequence that encodes such a viral polypeptide inhibitor. In some embodiments, an inhibitor of a NF-κB pathway to be delivered is or comprises an agent (e.g., a polypeptide-based or nucleic acid-based agent) that inhibits activity and/or formation of IκB kinase (IKK) complex. For example, in some embodiments, such an agent that inhibits activity and/or formation of IKK complex is or comprises an agent (e.g., a polypeptide-based or nucleic acid-based agent) that binds to and/or inhibits activity and/or interaction of at least one of an IKKα subunit, an IKKβ subunit, and an IKKγ subunit. In some embodiments, an agent that binds to and/or inhibits activity and/or interaction of an IKKβ subunit is or comprises a Vaccinia Virus Protein B14 polypeptide (e.g., a wild-type B14 polypeptide) and/or a nucleic acid (e.g., an RNA oligonucleotide) comprising a sequence that encoding the same. In some embodiments, a Vaccinia Virus Protein B14 polypeptide includes an amino acid sequence that is at least about 80%, 85%, 90%, 95%, 96%, 97%, 98%, 99% or higher (including and up to 100%) identical to a wild-type Vaccinia Virus Protein B14. In some embodiments, an exemplary wild-type Vaccinia Virus protein B14 has the amino acid sequence of SEQ ID NO: 5. In some embodiments, an inhibitor of NF-κB signaling is or comprises a B14 polypeptide that comprises the amino acid sequence of SEQ ID NO: 5 or a nucleic acid (e.g., an RNA oligonucleotide) encoding such a B14 polypeptide.

In some embodiments involving a pharmaceutical composition that delivers an inhibitor of an IRF pathway, such an inhibitor of an IRF pathway may be or comprise a viral polypeptide, which in some embodiments, may be delivered as a viral polypeptide inhibitor, or in some embodiments, may be delivered as a nucleic acid (e.g., an RNA oligonucleotide) comprising a sequence that encodes such a viral polypeptide inhibitor. In some embodiments, an inhibitor of an IRF pathway to be delivered is or comprises an agent (e.g., a polypeptide-based or nucleic acid-based agent) that inhibits activity and/or formation of a complex comprising TANK-binding kinase 1 (TBK1) and IκB kinase ε (IKKε). For example, in some embodiments, such an agent that inhibits activity and/or formation of the TBK1/IKKε complex is or comprises an agent (e.g., a polypeptide-based or nucleic acid-based agent) that binds to and/or inhibits activity and/or interaction of DEAD box protein 3 (DDX3) with the TBK1/IKKε complex, which, for example, in some embodiments, may be or comprise a Vaccinia Virus Protein K7 polypeptide (e.g., a wild-type K7 polypeptide) and/or a nucleic acid (e.g., an RNA oligonucleotide) comprising a sequence that encoding the same. In some embodiments, a Vaccinia Virus Protein K7 polypeptide includes an amino acid sequence that is at least about 80%, 85%, 90%, 95%, 96%, 97%, 98%, 99% or higher (including and up to 100%) identical to a wild-type Vaccinia Virus Protein K7. In some embodiments, an exemplary wild-type Vaccinia Virus Protein K7 has the amino acid sequence of SEQ ID NO: 9. In some embodiments, an inhibitor of IRF signaling is or comprises a K7 polypeptide that comprises the amino acid sequence of SEQ ID NO: 9 and/or a nucleic acid (e.g., an RNA oligonucleotide) encoding such a K7 polypeptide.

In some embodiments involving a pharmaceutical composition that delivers an inhibitor of an IRF pathway, such an inhibitor of an IRF pathway is or comprises an inhibitor of a JAK-STAT pathway, which in some embodiments, may be delivered as a polypeptide inhibitor, or in some embodiments, may be delivered as a nucleic acid (e.g., an RNA oligonucleotide) comprising a sequence that encodes such a polypeptide inhibitor. In some embodiments, an inhibitor of a JAK-STAT pathway is or comprises an agent (e.g., a polypeptide-based or nucleic acid-based agent) that inhibits activity and/or formation of a complex comprising STAT1, STAT2, and IRF9. For example, in some embodiments, an agent that inhibits activity and/or formation of a STAT1/STAT2/IRF9 complex is or comprises an agent (e.g., a polypeptide-based or nucleic acid-based agent) that binds to and/or inhibits activity and/or interaction of at least one of STAT1, STAT2, and IRF9. In some embodiments, such an agent that inhibits activity and/or formation of a STAT1/STAT2/IRF9 complex is or comprises an agent (e.g., a polypeptide-based or nucleic acid-based agent) that binds to and/or inhibits activity of STAT2. In some embodiments, such an agent that binds to and/or inhibits activity of STAT2 is or comprises a Vaccinia Virus Protein C6 polypeptide (e.g., a wild-type C6 polypeptide) and/or a nucleic acid (e.g., an RNA oligonucleotide) comprising a sequence that encoding the same. In some embodiments, a Vaccinia Virus Protein C6 polypeptide includes an amino acid sequence that is at least about 80%, 85%, 90%, 95%, 96%, 97%, 98%, 99% or higher (including and up to 100%) identical to a wild-type Vaccinia Virus Protein C6. In some embodiments, an exemplary wild-type Vaccinia Virus protein C6 has the amino acid sequence of SEQ ID NO: 12. In some embodiments, an inhibitor of IRF signaling is or comprises a C6 polypeptide that comprises the amino acid sequence of SEQ ID NO: 12 and/or a nucleic acid (e.g., an RNA oligonucleotide) encoding such a C6 polypeptide.

In some embodiments involving a pharmaceutical composition that delivers at least one inhibitor of a NF-κB pathway and at least one inhibitor of an IRF pathway, such a pharmaceutical composition may be formulated to comprise one or more compositions that deliver a Vaccinia Virus Protein B14 polypeptide and Vaccinia Virus Protein K7 polypeptide. In some embodiments, such a pharmaceutical composition may be formulated to comprise one or more compositions that deliver a Vaccinia Virus Protein B14 polypeptide and Vaccinia Virus Protein C6 polypeptide. In some embodiments, such a pharmaceutical composition may be formulated to comprise one or more compositions that deliver a Vaccinia Virus Protein K7 polypeptide and Vaccinia Virus Protein C6 polypeptide. In some embodiments, such a pharmaceutical composition may be formulated to comprise one or more compositions that deliver a Vaccinia Virus Protein B14 polypeptide, a Vaccinia Virus Protein K7 polypeptide, and a Vaccinia Virus Protein C6 polypeptide.

In some embodiments, the present disclosure provides methods of using nucleic acid systems and/or compositions described herein. In some embodiments, a provided method comprises: contacting a target cell with at least one of: (a) an oligonucleotide (e.g., RNA oligonucleotide such as, e.g., mRNA) comprising a payload sequence; and (b) at least one composition that delivers an inhibitor of a NF-κB pathway (e.g., ones described herein) and/or an inhibitor of an IRF pathway (e.g., ones described herein), such that both (a) and (b) are delivered to the target cell. In some embodiments, a method comprises contacting a target cell with at least one of (a) and (b) such that the target cell is receiving a nucleic acid expression system (e.g., ones described herein).

In some embodiments, provided methods can be useful for enhancing expression and/or activity of a payload sequence in a target cell. In some embodiments, expression and/or activity of a payload sequence in a target cell is enhanced by at least 30% or more, as compared to the expression and/or activity of the payload sequence in a target cell in the absence of the composition that delivers an inhibitor of a NF-κB pathway and/or an inhibitor of an IRF pathway.

In some embodiments, provided methods can be useful for reducing immunogenicity of an oligonucleotide (e.g., an RNA oligonucleotide) comprising a payload sequence upon delivery to a target cell. In some embodiments, immunogenicity of an oligonucleotide (e.g., an RNA oligonucleotide) comprising a payload sequence upon delivery to a cell is reduced by at least 30% or more, as compared to the immunogenicity of the oligonucleotide (e.g., RNA oligonucleotide) comprising the payload sequence upon delivery to a target cell in the absence of the composition that delivers an inhibitor of a NF-κB pathway and/or an inhibitor of an IRF pathway. One of ordinary skill in the art will understand that immunogenicity of an oligonucleotide (e.g., RNA oligonucleotide) may be characterized by methods known in the art; for example, in some embodiments, immunogenicity of an RNA oligonucleotide comprising a payload sequence may be characterized by detecting level and/or activity of at least one or more pro-inflammatory cytokines (e.g., TNF-α or IL-6). In some embodiments, immunogenicity of an RNA oligonucleotide comprising a payload sequence is characterized by detecting degradation of the RNA oligonucleotide comprising a payload sequence upon delivery to the cell.

In some embodiments, provided methods can be useful for enhancing viability of a target cell upon receiving a nucleic acid expression system as described herein. In some embodiments, viability of a target cell upon receiving a nucleic acid expression system (e.g., ones described herein) is enhanced by at least 30% or more, as compared to the viability of a target cell upon contacting with an oligonucleotide (e.g., an RNA oligonucleotide) comprising the payload sequence in the absence of the composition that delivers an inhibitor of a NF-κB pathway and/or an inhibitor of an IRF pathway.

In some embodiments, provided methods can be useful for reducing non-specific toxicity induced in a target cell by an oligonucleotide (e.g., an RNA oligonucleotide) comprising a payload sequence. In some embodiments, non-specific toxicity induced in a target cell by an oligonucleotide comprising a payload sequence is reduced by at least 30% or more, as compared to the non-specific toxicity induced in a target cell by an oligonucleotide comprising the payload sequence in the absence of the composition that delivers an inhibitor of a NF-κB pathway and/or an inhibitor of an IRF pathway. In some embodiments, non-specific toxicity induced in a target cell by an RNA oligonucleotide comprising a payload sequence is reduced by at least 30% or more, as compared to the non-specific toxicity induced in a target cell by an RNA oligonucleotide comprising the payload sequence in the absence of the composition that delivers an inhibitor of a NF-κB pathway and/or an inhibitor of an IRF pathway.

In some embodiments, provided methods can be useful for enhancing persistence or uptake of an oligonucleotide (e.g., a RNA oligonucleotide) comprising a payload sequence in a target cell. In some embodiments, persistence or uptake of an oligonucleotide comprising a payload sequence in a target cell is enhanced by at least 30% or more, as compared to the persistence or uptake of an oligonucleotide comprising the payload sequence introduced into a target cell in the absence of the composition that delivers an inhibitor of a NF-κB pathway and/or an inhibitor of an IRF pathway. In some embodiments, persistence or uptake of an RNA oligonucleotide comprising a payload sequence in a target cell is enhanced by at least 30% or more, as compared to the persistence or uptake of an RNA oligonucleotide comprising the payload sequence introduced into a target cell in the absence of the composition that delivers an inhibitor of a NF-κB pathway and/or an inhibitor of an IRF pathway.

In some embodiments of any provided methods described herein, a target cell may be previously contacted at least one by one or more synthetic oligonucleotides (e.g., RNA oligonucleotides). In some embodiments, a target cell in a provided method may be repeatedly contacted with an oligonucleotide (e.g., an RNA oligonucleotide) comprising a payload sequence, wherein the payload sequence may be the same or different in each contact.

In some embodiments of any provided methods described herein, a target cell may be concurrently receiving an oligonucleotide (e.g., RNA oligonucleotide) comprising a payload sequence and at least one composition that delivers an inhibitor of a NF-κB pathway and/or an inhibitor of an IRF pathway.

In some embodiments of any provided methods described herein, a target cell is receiving an oligonucleotide (e.g., RNA oligonucleotide) comprising a payload sequence and at least one composition that delivers an inhibitor of a NF-κB pathway and/or an inhibitor of an IRF pathway separately or in a sequential manner. In some embodiments, a target cell is receiving an oligonucleotide (e.g., an RNA oligonucleotide) comprising a payload sequence and at least one composition that delivers an inhibitor of a NF-κB pathway and/or an inhibitor of an IRF pathway separately within 24 hours or less.

In some embodiments where a target cell is receiving one or more compositions that deliver an inhibitor of a NF-κB pathway and an inhibitor of an IRF pathway, such an inhibitor of a NF-κB pathway and an inhibitor of an IRF pathway may be concurrently delivered to the target cell. Alternatively, such an inhibitor of a NF-κB pathway and an inhibitor of an IRF pathway may be separately or sequentially delivered to the target cell

In some embodiments, methods provided herein can be applied to a mammalian subject, for example, where a target cell is present in a mammalian subject. In some such embodiments where a target cell is present in a mammalian subject, the contacting step of a provided method comprises administering to the mammalian subject an oligonucleotide (e.g., an RNA oligonucleotide) comprising a payload sequence. In some embodiments, the contacting step of a provided method comprises administering to a mammalian subject at least one composition that delivers an inhibitor of a NF-κB pathway and/or an inhibitor of an IRF pathway. In some embodiments, a mammalian subject may be previously administered one or more synthetic oligonucleotides (e.g., RNA oligonucleotides). In some embodiments, a mammalian subject in a provided method may be repeatedly administered an oligonucleotide (e.g., an RNA oligonucleotide) comprising a payload sequence, wherein the payload sequence may be the same or different in each administration.

Also within the scope of the present disclosure are kits comprising a nucleic acid expression system as described herein, or one or more components thereof. For example, a kit of one aspect provided herein comprises: (a) a container including an oligonucleotide (e.g., a RNA oligonucleotide) comprising a payload sequence, and (b) at least one container including at least one composition that delivers an inhibitor of a NF-κB pathway and/or an inhibitor of an IRF pathway. In some embodiments, such a provided kit comprises at least one container including at least one composition that delivers an inhibitor of a NF-κB pathway and an inhibitor of an IRF pathway. In some embodiments, such a provided kit comprises at least one container including a composition that delivers one or more inhibitors of a NF-κB pathway and one or more inhibitors of an IRF pathway. In some embodiments, such a provided kit comprises at least two containers, wherein a first container includes at least one composition that delivers one or more inhibitors of a NF-κB pathway and a second container includes at least one composition that delivers one or more inhibitors of an IRF pathway.

A kit of another aspect provided herein comprises (a) a container including at least one composition that delivers an inhibitor of a NF-κB pathway; and (b) a container including at least one composition that delivers an inhibitor of an IRF pathway.

These, and other aspects encompassed by the present disclosure, are described in more detail below and in the claims.

### BRIEF DESCRIPTION OF THE DRAWINGS

FIG. 1 depicts activation of a NF-κB luciferase reporter system in the presence of various candidate inhibitors of a NF-κB pathway (as labeled on horizontal axis). Relative levels of NF-κB reporter expression are shown, normalized to cell viability.

FIGS. 2A-2D depict characterization data of cells contacted with at least one composition that delivers an inhibitor of an IRF pathway or in combination with various candidate inhibitors of a NF-κB pathway (as labeled on horizontal axis). FIG. 2A depicts activation of an IRF luciferase reporter system. Relative levels of IRF reporter expression are shown, normalized to cell viability. FIG. 2B depicts activation of a NF-κB luciferase reporter system. Relative levels of NF-κB reporter expression are shown, normalized to cell viability.

FIG. 2C depicts viability of cells transfected with indicated compositions. FIG. 2D depicts FLuc expression levels in cells transfected with indicated compositions.

FIG. 3 provides a schematic representation of signaling pathways of certain innate immune sensors converging on a NF-κB pathway and an IRF pathway.

FIG. 4 provides schematic representations of interaction of NF-κB, IRF, and JAK/STAT signaling. Panel A depicts a schematic of IFN gene expression with IFNα under the control of NF-κB sites and IFNβ under the control of combination of NF-κB sites and Interferon Stimulated Response Element (ISRE) sites. Panel B provides a schematic representation of canonical interferon and JAK/STAT signaling pathway.

### CERTAIN DEFINITIONS

About or approximately: As used herein, the terms “about” and “approximately,” when used herein in reference to a value, refers to a value that is similar, in context to the referenced value. In general, those skilled in the art, familiar with the context, will appreciate the relevant degree of variance encompassed by “about” or “approximately” in that context. For example, in some embodiments, the term “about” or “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to administration of a composition to a subject to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Co-delivery: As used herein, the term “co-delivery” refers to use of both an oligonucleotide (e.g., an RNA oligonucleotide) comprising a payload sequence and at least one composition that delivers one or more inhibitors of a NF-κB pathway and/or one or more inhibitors of an IRF pathway. The combined use of an oligonucleotide (e.g., an RNA oligonucleotide) comprising a payload sequence and at least one composition that delivers one or more inhibitors of a NF-κB pathway and/or one or more inhibitors of an IRF pathway may be performed concurrently or separately (e.g., sequentially in any order). In some embodiments of a pharmaceutical composition described herein, both an oligonucleotide (e.g., an RNA oligonucleotide) comprising a payload sequence and at least one composition that delivers one or more inhibitors of a NF-κB pathway and/or one or more inhibitors of an IRF pathway may be combined in one pharmaceutically-acceptable carrier, or they may be placed in separate carriers and delivered to a target cell or administered to a subject at different times. Each of these situations is contemplated as falling within the meaning of “co-delivery” or “co-administration” or “combination,” provided that both an oligonucleotide (e.g., an RNA oligonucleotide) comprising a payload sequence and at least one composition that delivers one or more inhibitors of a NF-κB pathway and/or one or more inhibitors of an IRF pathway are delivered or administered sufficiently close in time that there is at least some temporal overlap in biological effect(s) generated by each on a target cell or a subject being treated.

Complementary: As used herein, the term “complementary” refers to nucleotides or nucleotide sequences that base-pair according to the standard Watson-Crick complementary rules (adenine “A” base pairs with thymine “T”, and guanine “G” base pairs with cytosine “C”). Nucleotide sequences that are “100% complementary” or which exhibit “100% complementarity” are nucleotide sequences which base-pair with one another across the entirety of at least one of the two nucleotide sequences. An oligonucleotide can be “100% complementary” to a template polynucleotide that is longer than the oligonucleotide (i.e., the oligonucleotide is “100% complementary” to the template polynucleotide if the entire sequence of the oligonucleotide base-pairs with a portion of the template polynucleotide). However, nucleic acid sequences that are “complementary” need not be 100% complementary. Generally, the term “complementary” with respect to two or more nucleic acid sequences refers to there being sufficient complementarity across the two nucleic acid sequences such that they hybridize in stringent conditions and/or at temperatures used during annealing phases of amplification methods, e.g., PCR or LCR.

Delivery/contacting: As used interchangeably herein, the term “delivery,” “delivering,” or “contacting” refers to introduction of an oligonucleotide (e.g., RNA oligonucleotide) into a target cell (e.g., cytosol of a target cell). A target cell can be cultured in vitro or ex vivo or be present in a subject (in vivo). Methods of introducing an oligonucleotide (e.g., an RNA oligonucleotide) into a target cell can vary with in vitro, ex vivo, or in vivo applications. In some embodiments, an oligonucleotide (e.g., an RNA oligonucleotide) can be introduced into a target cell in a cell culture by in vitro transfection. In some embodiments, an oligonucleotide (e.g., an RNA oligonucleotide) can be introduced into a target cell via delivery vehicles (e.g., nanoparticles, liposomes, and/or complexation with a cell-penetrating agent). In some embodiments, an oligonucleotide (e.g., an RNA oligonucleotide) can be introduced into a target cell in a subject by administering an oligonucleotide to a subject.

Homology: As used herein, the term “homology” or “homolog” refers to the overall relatedness between oligonucleotide molecules (e.g., DNA molecules and/or RNA molecules) and/or between polypeptide molecules. In some embodiments, oligonucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, oligonucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Identity: As used herein, the term “identity” refers to the overall relatedness between oligonucleotide molecules (e.g., DNA molecules and/or RNA molecules) and/or between polypeptide molecules. In some embodiments, oligonucleotide molecules (e.g., DNA molecules and/or RNA molecules) and/or between polypeptide molecules are considered to be “substantially identical” to one another if their sequences are at least 25%, 30%, 35%, 40%, 45%, 50%, 55%, 60%, 65%, 70%, 75%, 80%, 85%, 90%, 95%, 96%, 97%, 98%, or 99% identical. Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, at least 96%, at least 97%, at least 98%, at least 99%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller, 1989, which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Inhibit: The term “inhibit” or “inhibition” in the context of modulating a signaling pathway and/or expression and/or activity level of a target within a signaling pathway is not limited to only total inhibition. Thus, in some embodiments, partial inhibition or relative reduction is included within the scope of the term “inhibition.” In some embodiments, the term refers to a reduction in activation of a signaling pathway and/or expression and/or activity level of a target within a signaling pathway to a level that is reproducibly and/or statistically significantly lower than an initial or other appropriate reference level, which may, for example, be a baseline level of a signaling pathway and/or expression and/or activity level of a target within a signaling pathway in the absence or prior to administration of an inhibitor of a target signaling pathway (e.g., a NF-κB pathway and/or an IRF pathway). In some embodiments, the term refers to a reduction in activation of a signaling pathway and/or expression and/or activity level of a target within a signaling pathway to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of a signaling pathway and/or expression and/or activity level of a target within a signaling pathway in the absence or prior to administration of an inhibitor of a target signaling pathway (e.g., a NF-κB pathway and/or an IRF pathway).

Inhibitor: As used herein, the term “inhibitor” refers to an agent whose presence, level, or degree correlates with decreased level or activity of a target. In some embodiments, an inhibitor may be act directly (in which case it exerts its influence directly upon its target, for example by binding to the target); in some embodiments, an inhibitor may act indirectly (in which case it exerts its influence by interacting with and/or otherwise altering a regulator of a target, e.g., a component of a pathway in which the target is involved, so that level and/or activity of the target is reduced). In some embodiments, an inhibitor is one whose presence or level correlates with a target level or activity that is reduced relative to a particular reference level or activity (e.g., that observed under appropriate reference conditions, such as presence of a known inhibitor, or absence of the inhibitor as disclosed herein, etc.). In some embodiments, an inhibitor is a polypeptide agent. In some embodiments, an inhibitor is a nucleic acid agent. In some embodiments, an inhibitor of a NF-κB pathway refers to an agent whose presence, level, or degree correlates with a decreased level or activity of at least one component involved in or associated with NF-κB activation pathway. In some embodiments, an inhibitor of an IRF pathway refers to an agent whose presence, level, or degree correlates with a decreased level or activity of at least one component involved in or associated with IRF activation pathway.

Non-specific toxicity: In context of introduction of an oligonucleotide, e.g., an oligonucleotide, e.g., an RNA oligonucleotide, comprising a payload sequence, into a target cell, the term “non-specific toxicity” refers to cell toxicity induced by an oligonucleotide (e.g., an RNA oligonucleotide) independent of a function and/or activity of a payload sequence. For example, when an oligonucleotide (e.g., an RNA oligonucleotide) comprising a non-cytotoxic payload sequence causes comparable cell death (an exemplary indicator of cell toxicity) to that caused by an oligonucleotide (e.g., an RNA oligonucleotide) comprising a cytotoxic payload sequence, the cell death (or cell toxicity) is nonspecific because it is independent of the cytotoxic nature of a payload sequence. In some embodiments, “non-specific toxicity” also refers to cell toxicity induced in any cells including, e.g., both target and non-target cells (e.g., normal healthy cells), rather than induced in target cells only.

Nucleic acid/Oligonucleotide: As used herein, the terms “nucleic acid” and “oligonucleotide” are used interchangeably, and refer to a polymer of at least 3 nucleotides or more. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleotide: As used herein, the term “nucleotide” refers to its art-recognized meaning. When a number of nucleotides is used as an indication of size, e.g., of an RNA oligonucleotide, a certain number of nucleotides refers to the number of nucleotides on a single strand, e.g., of an RNA oligonucleotide.

Ortholog: As used herein, the term “ortholog” refers to its art-recognized meaning. An ortholog is a homology subtype, which is a polypeptide from one species that is a functional counterpart of a polypeptide from a reference species. Typically, sequence differences among orthologs are the result of speciation. For example, innate immune repressor polypeptides that inhibit NF-κB pathway and/or IRF pathway from different virus species are considered to be orthologs. In some embodiments, oligonucleotide molecules (e.g., DNA molecules and/or RNA molecules) and/or polypeptide molecules from different species are considered to be “orthologous” to one another if their sequences are functionally related and at least 15%, 20%, 25%, 30%, 35%, 40%, 45%, 50%, 55%, 60%, 65%, 70%, 75%, 80%, 85%, 90%, 95%, or 99% identical. In some embodiments, oligonucleotide molecules (e.g., DNA molecules and/or RNA molecules) and/or polypeptide molecules from different species are considered to be “orthologous” to one another if their sequences are functionally related and at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Polypeptide: The term “polypeptide”, as used herein, generally has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof.

RNA oligonucleotide: As used herein, the term “RNA oligonucleotide” refers to an oligonucleotide of ribonucleotides. In some embodiments, an RNA oligonucleotide is single stranded. In some embodiments, an RNA oligonucleotide is double stranded. In some embodiments, an RNA oligonucleotide comprises both single and double stranded portions. In some embodiments, an RNA oligonucleotide can comprise a backbone structure as described in the definition of “Nucleic acid/Oligonucleotide” above. An RNA oligonucleotide can be a regulatory RNA (e.g., siRNA, microRNA, etc.), or a messenger RNA (mRNA) oligonucleotide. In some embodiments where an RNA oligonucleotide is a mRNA oligonucleotide. In some embodiments where an RNA oligonucleotide is a mRNA oligonucleotide, a RNA oligonucleotide typically comprises at its 3′ end a poly(A) region. In some embodiments where an RNA oligonucleotide is a mRNA oligonucleotide, an RNA oligonucleotide typically comprises at its 5′ end an art-recognized cap structure, e.g., for recognizing and attachment of a mRNA to a ribosome to initiate translation. In some embodiments, a RNA oligonucleotide is a synthetic RNA oligonucleotide. Synthetic RNA oligonucleotides include RNA oligonucleotides that are synthesized in vitro (e.g., by enzymatic synthesis methods and/or by chemical synthesis methods).

Subject: As used herein, the term “subject” refers an organism, typically a mammal (e.g., a human). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered. In some embodiments, a subject is an individual (e.g., a human) who has undergone an RNA oligonucleotide therapy or a gene therapy at least once or more. In some embodiments, a subject is an individual (e.g., a human) who is undergoing an RNA oligonucleotide therapy or a gene therapy.

Synthetic: As used herein, the term “synthetic” refers to an entity that is artificial, or that is made with human intervention, or that results from synthesis rather than naturally occurring. For example, in some embodiments, a synthetic oligonucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic oligonucleotide refers to a nucleic acid molecule (e.g., an RNA oligonucleotide) that is produced by in vitro transcription using a template.

Standard techniques may be used for recombinant DNA, oligonucleotide synthesis, e.g., mRNA synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2nd ed., Cold Spring Harbor Laboratory Press, Cold Spring Harbor, N.Y. (1989)), which is incorporated herein by reference for any purpose.

### DETAILED DESCRIPTION OF CERTAIN EMBODIMENTS

mRNA vectors have emerged as a robust therapeutic modality for a variety of applications, including, e.g., gene therapy, vaccine, and/or immune-oncology. However, there are challenges of delivering mRNAs into subjects, e.g., high immunogenicity associated with foreign RNAs. For example, since many viruses generate either fully double stranded RNA (dsRNA) or single-stranded RNA (ssRNA) with secondary structures during their life cycles, humans have evolved sophisticated innate immune sensors to enable both professional antigen-presenting cells (APCs) and non-immune cells to detect double-stranded and improperly capped RNAs. Accordingly, there remains a need to develop methods and compositions for improving RNA therapeutics, e.g., so that the RNA does not activate an innate immune response, yet is still efficiently recognized by a translational machinery in a cell.

Prior approaches of reducing immunogenicity of synthetic RNAs focused on inhibiting individual innate immune sensors and/or secreted proteins associated with an anti-viral state, examples of which include chemically modified nucleotides (Karikó et al., Mol. Ther. 2008; 16(11):1833-40) that reduce the ability of sensors to detect synthetic RNA, and viral inhibitors of type I interferons (Warren et al., Cell Stem Cell 2010) or RNA sensors (Beissert et al., Human Gene Ther. 2017; 28(12):1138-1146). The contents of each of the aforementioned references are incorporated by reference in their entirety for purposes described herein. The present disclosure encompasses a recognition that signaling through certain innate immune sensors (e.g., TLR3, TLR7, TLR8, MDA5, and/or RIG-I) converges on two signaling pathways: Nuclear Factor kappa-light chain enhancer of activated B cells (NF-κB) and Interferon Regulatory Factors (IRF), which in some embodiments, can jointly induce an innate immune response and/or an anti-viral state in a target cell.

The present disclosure is based, at least in part, on the insight that targeting a signaling “bottleneck” between innate immune sensors and downstream amplification cascades can be more efficient in reducing immunogenicity associated with RNA oligonucleotide delivery, as compared to certain prior technologies that directly target individual innate immune sensors. For example, the present disclosure provides, among other things, a unique approach for reducing immunogenicity of RNA oligonucleotides by targeting one or more signaling components downstream of certain innate immune sensors that converge on a NF-κB pathway and/or IRF pathway, rather than the innate immune sensors themselves or secreted proteins associated with an anti-viral state. Without wishing to be bound by theory, the present disclosure provides the insight that targeting such a signaling “bottleneck” (e.g., through inhibition of NF-κB and/or IRF) may provide robust innate immune suppression.

The present disclosure is based, at least in part, on the insight that co-delivery (e.g., to a subject or target cell) of an oligonucleotide (e.g., an RNA oligonucleotide such as a messenger RNA (mRNA)) comprising a payload sequence with a composition that inhibits NF-κB pathway signaling and/or IRF pathway signaling may reduce innate immunity-triggered suppression of protein translation and/or reduce degradation of delivered oligonucleotides (e.g., RNA oligonucleotides). In some embodiments, a reduction in innate immunity-triggered suppression of protein translation and/or degradation of delivered oligonucleotides (e.g., RNA oligonucleotides) can, in turn, improve expression of an oligonucleotide (e.g., an RNA oligonucleotide such as mRNA) in target cells.

Accordingly, the present disclosure, among other things, provides nucleic acid expression systems and compositions for delivery of an oligonucleotide, e.g., an RNA oligonucleotide (e.g., mRNA), comprising a payload sequence and at least one composition that delivers an inhibitor of a NF-κB pathway and/or an inhibitor of an IRF pathway. Methods for using such nucleic acid expression systems and compositions are also provided herein.

### I. Nucleic Acid Expression Systems

In one aspect, the present disclosure provides nucleic acid expression systems for expressing payload sequences in cells. In some embodiments, provided is a nucleic acid expression system includes one or more compositions that deliver one or more inhibitors of a NF-κB pathway and/or one or more inhibitors of an IRF pathway. In some embodiments, a nucleic acid expression system includes a nucleic acid or oligonucleotide (e.g., RNA oligonucleotide) comprising a payload sequence and at least one composition that delivers at least one or more inhibitors of a NF-κB pathway and/or at least one or more inhibitors of an IRF pathway. In some embodiments, a nucleic acid expression system includes a nucleic acid or oligonucleotide (e.g., RNA oligonucleotide) comprising a payload sequence and at least one composition that delivers at least one or more NF-κB pathway inhibitor. In some embodiments, a nucleic acid expression system includes a nucleic acid or oligonucleotide (e.g., RNA oligonucleotide) comprising a payload sequence and at least one composition that delivers one or more inhibitors of an IRF pathway. In some embodiments, a nucleic acid expression system includes a nucleic acid or oligonucleotide (e.g., RNA oligonucleotide) comprising a payload sequence and one or more compositions that, when combined, deliver at least one inhibitor of a NF-κB pathway and at least one inhibitor of an IRF pathway. In some embodiments, a nucleic acid expression system includes a nucleic acid or oligonucleotide (e.g., RNA oligonucleotide) comprising a payload sequence and one or more compositions that, when combined, deliver at least one inhibitor of a NF-κB pathway and two or more inhibitors of an IRF pathway. In some embodiments, a nucleic acid expression system includes a nucleic acid or oligonucleotide (e.g., RNA oligonucleotide) comprising a payload sequence and one or more compositions that, when combined, deliver at least two inhibitors of a NF-κB pathway and at least one inhibitor of an IRF pathway.

In some embodiments, a nucleic acid expression system includes one or more compositions that, when combined, deliver (i) at least one nucleic acid (e.g., RNA oligonucleotide) comprising a payload sequence and (ii) at least one inhibitor of a NF-κB pathway and/or at least one inhibitor of an IRF pathway. In some embodiments, a nucleic acid expression system includes two or more compositions that, when combined, deliver (i) at least one nucleic acid (e.g., RNA oligonucleotide) comprising a payload sequence and (ii) at least one inhibitor of a NF-κB pathway and/or at least one inhibitor of an IRF pathway.

In some embodiments where a nucleic acid expression system includes a nucleic acid (e.g., an RNA oligonucleotide) comprising a payload sequence, such a nucleic acid may be present in a separate composition from one or more compositions that deliver at least one inhibitor of a NF-κB pathway and/or at least one inhibitor of an IRF pathway. For example, in some embodiments, a nucleic acid expression system includes (i) a composition comprising a nucleic acid (e.g., RNA oligonucleotide) that includes a payload sequence and (ii) one or more compositions that deliver at least one inhibitor of a NF-κB pathway and at least one inhibitor of an IRF pathway.

In some embodiments, a nucleic acid expression system comprises: (i) a nucleic acid (e.g., an RNA oligonucleotide such as, e.g., mRNA) comprising a payload sequence, and (ii) one or more compositions that deliver at least one or more inhibitors of a NF-κB pathway. For example, in some such embodiments, one or more compositions that deliver at least one or more inhibitors of a NF-κB pathway include a polypeptide inhibitor of a NF-κB pathway and/or a nucleic acid (e.g., an RNA oligonucleotide) comprising a sequence that encodes an inhibitor of a NF-κB pathway. In some embodiments, one or more compositions that deliver at least one or more inhibitors of a NF-κB pathway include an RNA oligonucleotide comprising a sequence that encodes an inhibitor of a NF-κB pathway.

In some embodiments, a nucleic acid expression system comprises: (i) a nucleic acid (e.g., an RNA oligonucleotide such as, e.g., mRNA) comprising a payload sequence, and (ii) one or more compositions that deliver at least one or more inhibitors of an IRF pathway. For example, in some such embodiments, one or more compositions that deliver at least one or more inhibitors of an IRF pathway include a polypeptide inhibitor of an IRF pathway and/or a nucleic acid (e.g., an RNA oligonucleotide) comprising a sequence that encodes an inhibitor of an IRF pathway. In some embodiments, one or more compositions that deliver at least one or more inhibitors of an IRF pathway include an RNA oligonucleotide comprising a sequence that encodes an inhibitor of an IRF pathway.

In some embodiments, a nucleic acid expression system comprises: (i) a nucleic (e.g., an RNA oligonucleotide such as, e.g., mRNA) comprising a payload sequence, and (ii) one or more compositions that deliver at least one or more inhibitors of a NF-κB pathway and at least one or more inhibitors of an IRF pathway. For example, in some such embodiments, one or more compositions that deliver at least one or more inhibitors of a NF-κB pathway and at least one or more inhibitors of an IRF pathway include a polypeptide inhibitor of a NF-κB pathway and a polypeptide inhibitor of an IRF pathway. In some embodiments, one or more compositions that deliver at least one or more inhibitors of a NF-κB pathway and at least one or more inhibitors of an IRF pathway include a polypeptide inhibitor of a NF-κB pathway and a nucleic acid (e.g., an RNA oligonucleotide) comprising a sequence that encodes an inhibitor of an IRF pathway. In some embodiments, one or more compositions that deliver at least one or more inhibitors of a NF-κB pathway and at least one or more inhibitors of an IRF pathway include a nucleic acid (e.g., an RNA oligonucleotide) comprising a sequence that encodes an inhibitor of a NF-κB pathway and a polypeptide inhibitor of an IRF pathway. In some embodiments, one or more compositions that deliver at least one or more inhibitors of a NF-κB pathway and at least one or more inhibitors of an IRF pathway include a nucleic acid (e.g., an RNA oligonucleotide) comprising a sequence that encodes an inhibitor of a NF-κB pathway and a nucleic acid (e.g., an RNA oligonucleotide) comprising a sequence that encodes an inhibitor of an IRF pathway. In some embodiments, one or more compositions that deliver at least one or more inhibitors of a NF-κB pathway and at least one or more inhibitors of an IRF pathway include an RNA oligonucleotide comprising a sequence that encodes an inhibitor of a NF-κB pathway and an RNA oligonucleotide comprising a sequence that encodes an inhibitor of an IRF pathway.

In some embodiments, a nucleic acid expression system includes one or more compositions that deliver (i) a polypeptide inhibitor of a NF-κB pathway or a nucleic acid comprising a sequence that encodes an inhibitor of a NF-κB pathway and/or (ii) polypeptide inhibitor of an IRF pathway or a nucleic acid comprising a sequence that encodes an inhibitor of an IRF pathway. In some embodiments, a nucleic acid expression system includes a composition that delivers one or more polypeptide inhibitors of a NF-κB pathway and/or one or more nucleic acids comprising a sequence that encodes an inhibitor of a NF-κB pathway. In some embodiments, a nucleic acid expression system includes a composition that delivers one or more polypeptide inhibitors of an IRF pathway and/or one or more nucleic acids comprising a sequence that encodes an inhibitor of an IRF pathway. In some embodiments, a nucleic acid expression system includes a nucleic acid comprising a payload sequence and a composition that delivers an inhibitor of a NF-κB pathway and an inhibitor of an IRF pathway, wherein the inhibitors are or comprise a polypeptide inhibitor and/or a nucleic acid encoding an inhibitor.

In some embodiments where inhibitor(s) of a NF-κB pathway and/or inhibitor(s) of an IRF pathway are delivered as a nucleic acid-based agent, a composition that delivers such inhibitors include a nucleic acid construct encoding an inhibitor of a NF-κB pathway and/or a nucleic acid construct encoding an inhibitor of an IRF pathway.

In some embodiments, a nucleic acid expression system includes one or more compositions that deliver an inhibitor of a NF-κB pathway and an inhibitor of an IRF pathway, where the one or more compositions together include two or more nucleic acid constructs encoding at least one inhibitor (e.g., one, two, or more) of a NF-κB pathway and at least one inhibitor (e.g., one, two, or more) of an IRF pathway. In some embodiments, a composition delivers an inhibitor of a NF-κB pathway and an inhibitor of an IRF pathway, where said composition includes a nucleic acid construct encoding an inhibitor of a NF-κB pathway and a separate nucleic acid construct encoding an inhibitor of an IRF pathway. In some embodiments, a composition delivers an inhibitor of a NF-κB pathway and an inhibitor of an IRF pathway, where said composition includes a nucleic acid construct encoding both an inhibitor of a NF-κB pathway and an inhibitor of an IRF pathway.

In some embodiments where a nucleic acid expression system includes one or more compositions that deliver at least one or more inhibitors of a NF-κB pathway and at least one or more inhibitors of an IRF pathway, such inhibitors of a NF-κB pathway and of an IRF pathway can be provided in the same composition or in separate compositions. For example, in some embodiments, a nucleic acid expression system includes two or more compositions that, when combined, deliver (i) one or more inhibitors of a NF-κB pathway and (ii) one or more inhibitors of an IRF pathway. In some embodiments, a nucleic acid expression system includes a composition that delivers at least one inhibitor of a NF-κB pathway and a separate composition that delivers at least one inhibitor of an IRF pathway. In some embodiments, a nucleic acid expression system includes a composition that delivers at least one inhibitor of a NF-κB pathway and one or more additional compositions that deliver two or more inhibitors of an IRF pathway. In some embodiments, a nucleic acid expression system includes one or more compositions that deliver two or more inhibitors of a NF-κB pathway and one or more additional compositions that deliver one or more inhibitors of an IRF pathway.

In some embodiments, a nucleic acid expression system includes one or more compositions that deliver (i) a nucleic acid comprising a sequence that encodes an inhibitor of a NF-κB pathway and/or (ii) a nucleic acid comprising a sequence that encodes an inhibitor of an IRF pathway. In some embodiments, a nucleic acid comprising a sequence that encodes an inhibitor of NF-κB pathway and/or IRF pathway is a RNA oligonucleotide. In some embodiments, a RNA oligonucleotide that encodes an inhibitor of NF-κB pathway and/or IRF pathway is a mRNA oligonucleotide. In some embodiments, a RNA oligonucleotide that encodes an inhibitor of NF-κB pathway and/or IRF pathway is a regulatory RNA (e.g., siRNA, microRNA, etc.).

In some embodiments, a nucleic acid expression system includes a nucleic acid (e.g., an RNA oligonucleotide) comprising a payload sequence and one or more sequences that encode an inhibitor of a NF-κB pathway and/or an inhibitor of an IRF pathway. In some embodiments, a nucleic acid expression system includes an RNA oligonucleotide comprising at least one internal ribosomal entry site (IRES) between a payload sequence and one or more sequences that encodes an inhibitor of a NF-κB pathway and/or an inhibitor of an IRF pathway. In some embodiments, a payload sequence, a sequence encoding an inhibitor of a NF-kB pathway, a sequence encoding an inhibitor of an IRF pathway, or a combination thereof can be under the control of an IRES. In some embodiments, a nucleic acid (e.g., an RNA oligonucleotide) provided in a nucleic acid expression system can include the following component sequences in the direction from 5′ to 3′: a cap-UTR-payload-IRES-inhibitor-UTR-polyadenyl sequence (pA), wherein the component “inhibitor” refers to one or more sequences encoding an inhibitor of a NF-kB pathway and/or an inhibitor of an IRF pathway. In some embodiments, a nucleic acid (e.g., an RNA oligonucleotide) provided in a nucleic acid expression system can include the following component sequences in the direction from 5′ to 3′: a cap-UTR-inhibitor-IRES-payload-UTR-pA, wherein the component “inhibitor” refers to one or more sequences encoding an inhibitor of a NF-kB pathway and/or an inhibitor of an IRF pathway. In some embodiments, a nucleic acid (e.g., an RNA oligonucleotide) provided in a nucleic acid expression system can include the following component sequences in the direction from 5′ to 3′: IRES-payload-IRES-inhibitor-UTR-pA, wherein the component “inhibitor” refers to one or more sequences encoding an inhibitor of a NF-kB pathway and/or an inhibitor of an IRF pathway. In some embodiments, a nucleic acid (e.g., an RNA oligonucleotide) provided in a nucleic acid expression system can include the following component sequences in the direction from 5′ to 3′: IRES-inhibitor-IRES-payload-UTR-pA, wherein the component “inhibitor” refers to one or more sequences encoding an inhibitor of a NF-kB pathway and/or an inhibitor of an IRF pathway.

In some embodiments, a nucleic acid expression system includes a nucleic acid (e.g., an RNA oligonucleotide) comprising a payload sequence and one or more nucleic acids encoding an inhibitor of a NF-κB pathway and/or an inhibitor of an IRF pathway. In some embodiments, a nucleic acid expression system includes an RNA oligonucleotide comprising a payload sequence and one or more RNA oligonucleotides encoding an inhibitor of a NF-κB pathway and/or an inhibitor of an IRF pathway. In some embodiments, a nucleic acid expression system includes an RNA oligonucleotide comprising a payload sequence and an RNA oligonucleotide encoding an inhibitor of a NF-κB pathway. In some embodiments, a nucleic acid expression system includes an RNA oligonucleotide comprising a payload sequence and an RNA oligonucleotide encoding an inhibitor of an IRF pathway. In some embodiments, a nucleic acid expression system includes an RNA oligonucleotide comprising a payload sequence and an RNA oligonucleotide encoding an inhibitor of a NF-κB pathway and an inhibitor of an IRF pathway.

In some embodiments, a nucleic acid expression system includes at least one or more nucleic acids (e.g., RNA oligonucleotide(s)) comprising a payload sequence (e.g., as described herein) and least one composition that delivers at least one inhibitor of a NF-κB pathway (e.g., as described herein) and/or at least one inhibitor of an IRF pathway (e.g., as described herein). In some such embodiments, a nucleic acid expression system includes a plurality of (e.g., 2, 3, 4, 5, 6, 7, 8, 9, 10, or more) nucleic acids (e.g., RNA oligonucleotides), each comprising one or more payload sequences, which may be directed to the same target or different targets. In some embodiments, a nucleic acid expression system includes a plurality of (e.g., at least 2, at least 3, at least 4, at least 5, at least 6, at least 7, at least 8, at least 9, at least 10, or more) compositions that each deliver a distinct inhibitor of a NF-κB pathway. In some embodiments, a nucleic acid expression system includes a plurality of (e.g., at least 2, at least 3, at least 4, at least 5, at least 6, at least 7, at least 8, at least 9, at least 10, or more) compositions that each deliver a distinct inhibitor of an IRF pathway. In some embodiments, a nucleic acid expression system includes a plurality of (e.g., at least 2, at least 3, at least 4, at least 5, at least 6, at least 7, at least 8, at least 9, at least 10, or more) compositions that, when combined, deliver at least one inhibitor of a NF-κB pathway and at least one inhibitor of an IRF pathway.

In some embodiments, inhibitor(s) of a NF-κB pathway and/or inhibitor(s) of an IRF pathway are delivered as nucleic acid-based agents. Accordingly, in some embodiments, a nucleic acid expression system includes at least one or more nucleic acid (e.g., an RNA oligonucleotide) comprising a payload sequence (e.g., as described herein) and least one nucleic acid (e.g., RNA oligonucleotide) comprising a sequence that encodes an inhibitor of a NF-κB pathway and/or an inhibitor of an IRF pathway (e.g., as described herein). In some such embodiments, a nucleic acid expression system includes a plurality of (e.g., 2, 3, 4, 5, 6, 7, 8, 9, 10, or more) nucleic acids (e.g., RNA oligonucleotides), each comprising one or more payload sequences, which may be directed to the same target or different targets. In some embodiments, a nucleic acid expression system includes a plurality of (e.g., at least 2, at least 3, at least 4, at least 5, at least 6, at least 7, at least 8, at least 9, at least 10, or more) nucleic acids (e.g., RNA oligonucleotides), each encoding a distinct inhibitor of a NF-κB pathway. In some embodiments, a nucleic acid expression system includes a plurality of (e.g., at least 2, at least 3, at least 4, at least 5, at least 6, at least 7, at least 8, at least 9, at least 10, or more) nucleic acids (e.g., RNA oligonucleotides) each encoding a distinct inhibitor of an IRF pathway. In some embodiments, a nucleic acid expression system includes a plurality of (e.g., at least 2, at least 3, at least 4, at least 5, at least 6, at least 7, at least 8, at least 9, at least 10, or more) nucleic acids (e.g., RNA oligonucleotides), which at least one encodes an inhibitor of a NF-κB pathway and at least one encodes an inhibitor of an IRF pathway.

In some embodiments involving RNA oligonucleotides (e.g., synthetic RNA oligonucleotides comprising a payload sequence and/or encoding an IRF inhibitor and/or a NF-κB inhibitor) of any aspects described herein), such RNA oligonucleotides are messenger RNA (mRNA) oligonucleotides. For example, in some embodiments, an RNA oligonucleotide comprising a payload sequence is a mRNA oligonucleotide. In some embodiments, an RNA oligonucleotide comprising a sequence that encodes an inhibitor of a NF-κB pathway and/or an inhibitor of an IRF pathway is a mRNA oligonucleotide.

In some embodiments, nucleic acids such as RNA oligonucleotides (e.g., comprising a payload sequence and/or encoding an IRF inhibitor and/or a NF-κB inhibitor) of any aspects described herein are synthetic oligonucleotides (e.g., synthetic RNA oligonucleotides). For example, in some embodiments, an RNA oligonucleotide comprising a payload sequence is a synthetic RNA oligonucleotide. In some embodiments, an RNA oligonucleotide comprising a sequence that encodes an inhibitor of a NF-κB pathway and/or an inhibitor of an IRF pathway is a synthetic RNA oligonucleotide Synthetic nucleic acids or oligonucleotides can be produced by any methods known in the art. For example, in some embodiments where synthetic RNA oligonucleotides are synthetic mRNA oligonucleotides, they can be produced, e.g., by in vitro transcription of a cDNA template, typically plasmid DNA (pDNA), using an RNA polymerase, e.g., a bacteriophage RNA polymerase. In some embodiments, a synthetic RNA oligonucleotide is produced by in vitro transcription of gBlock dsDNA.

In some embodiments, oligonucleotides (e.g., comprising a payload sequence and/or encoding an IRF inhibitor and/or a NF-κB inhibitor) of any aspects described herein can be in the context of a vector. Generally, vectors in the context of the present disclosure are capable of transferring or delivering oligonucleotide sequences to target cells. In some embodiments, a vector is a cloning vector. In some embodiments, a vector is an expression vector. In some embodiments, a vector is an integrating vector. In some embodiments, a vector is a non-viral vector. In some embodiments, a vector is a viral vector.

In some embodiments, a vector is a DNA vector. Various DNA vectors are known in the art; one skilled in the art reading the present disclosure will appreciate that suitable DNA vectors can be used in accordance with the present disclosure. In some embodiments, a vector is a non-viral DNA vector. In some embodiments, a vector is a viral DNA vector.

In some embodiments, a vector is an RNA vector. Various RNA vectors are known in the art; one skilled in the art reading the present disclosure will appreciate that suitable RNA vectors can be used in accordance with the present disclosure.

In some embodiments, a vector is a linearized vector. In some embodiments, a vector is a linear covalently closed (lcc) nucleic acid vector. In some embodiments, lcc vectors are DNA vectors. In some embodiments, lcc vectors are RNA vectors (e.g., an mRNA vector).

Payload Oligonucleotides

The present disclosure provides systems, compositions, and methods useful for the delivery of nucleic acids that include a payload sequence. Payload oligonucleotides of any aspects described herein are oligonucleotides that include a payload sequence. A payload sequence is generally a sequence of interest (e.g., comprising a sequence that encodes a target payload such as a target peptide or polypeptide) that is desired to be introduced into a cell, tissue, organ, organism, and/or system comprising cells.

In some embodiments, a payload sequence comprises a sequence that encodes a single target peptide or polypeptide. For example, in some embodiments, a payload sequence encodes a target polypeptide (e.g., an enzyme, cytokine, antibody, receptor, etc.). In some embodiments, a payload sequence comprises a sequence that encodes a plurality of (e.g., at least 2, at least 3, at least 4, at least 5, or above) target peptides or polypeptides. In some embodiments, a payload sequence comprises a sequence that encodes a fusion polypeptide and/or a chimeric polypeptide, e.g., a payload sequence encoding at least a portion of two or more peptides or polypeptides (e.g., a chimeric receptor). In some embodiments, a payload sequence comprises a sequence that encodes two or more polypeptides in the same oligonucleotide (e.g., two or more polypeptides are that controlled by the same or different regulatory elements).

In some embodiments, a payload sequence comprises a synthetic nucleic acid. For example, in some embodiments, payload oligonucleotides are DNA oligonucleotides that comprise a payload sequence. For example, in some embodiments, a DNA oligonucleotide comprising a payload sequence is a synthetic DNA oligonucleotide. In some embodiments, an oligonucleotide comprising a payload sequence is a viral DNA oligonucleotide. In some embodiments, an oligonucleotide comprising a payload sequence is a non-viral DNA oligonucleotide. In some embodiments, an oligonucleotide comprising a payload sequence is a single-stranded DNA (ssDNA) oligonucleotide. In some embodiments, an oligonucleotide comprising a payload sequence is a double-stranded DNA (dsDNA) oligonucleotide. In some embodiments, an oligonucleotide comprising a payload sequence is a DNA-RNA hybrid oligonucleotide.

In some embodiments, an oligonucleotide comprising a payload sequence is an RNA oligonucleotide (e.g., a mRNA). In some embodiments, an oligonucleotide comprising a payload sequence is a single-stranded RNA (ssRNA) oligonucleotide. In some embodiments, an oligonucleotide comprising a payload sequence is a double-stranded RNA (dsRNA) oligonucleotide.

In some embodiments, an RNA oligonucleotide comprising a payload sequence is a viral RNA oligonucleotide. In some embodiments, an RNA oligonucleotide comprising a payload sequence is a non-viral RNA oligonucleotide.

In some embodiments, an RNA oligonucleotide comprising a payload sequence is a synthetic RNA oligonucleotide. Synthetic RNA oligonucleotides can be produced by any methods known in the art. For example, in some embodiments where synthetic RNA oligonucleotides are synthetic mRNA oligonucleotides, they can be produced, e.g., by in vitro transcription of a DNA template.

In some embodiments, an RNA oligonucleotide comprising a payload sequence is a mRNA oligonucleotide. In some embodiments, a mRNA oligonucleotide comprises a target payload-encoding open reading frame (ORF), a poly(A) tail (e.g., at the 3′ end), and a “cap,” e.g., a 7-methyl-guanosine residue, for example, joined to the 5′-end via a 5′-5′ triphosphate.

In some embodiments of any aspects described, RNA oligonucleotides comprising a payload sequence include an extension sequence at their 5′ and/or 3′ ends. In some embodiments, RNA oligonucleotides comprising a payload sequence further comprise an additional element, including, but not limited to, spacers, binding motifs, etc.

In some embodiments, an oligonucleotide (e.g., RNA oligonucleotide) comprising a payload sequence comprises one or more of: a target-encoding region, a gene regulatory element, and a transcription terminator. Non-limiting examples of gene regulatory elements include promoters, transcriptional activators, enhancers, and polyadenylation signals. In some embodiments, a payload sequence comprises a target-encoding region, a gene regulatory element, and a transcription terminator, positioned relative to each other such that the target-encoding region is between the gene regulatory element and the transcription terminator.

In some embodiments, a target-encoding region encodes a gene product. In some embodiments, such a gene product is an RNA. In some embodiments, a target-encoding region encodes a polypeptide (such as a protein, such as a glycoprotein). In some embodiments, a target-encoding region encodes a fusion polypeptide and/or a chimeric polypeptide. In some embodiments, a target-encoding region encodes one gene product. In some embodiments, a target-encoding region encodes more than one gene product (e.g., 2, 3, 4, 5, 6, 7 or more gene products). In some embodiments, a target-encoding region encodes a regulatory RNA (e.g., a siRNA, microRNA, etc.).

In some embodiments, a payload sequence comprises one or more aptamer- or polypeptide-binding domains (e.g., transcription factor binding domains).

A payload sequence can be of any length, for example, between 2 and 100,000,000 nucleotides in length (or any integer value therebetween). In some embodiments, a payload sequence comprises at least 20 nucleotides, at least 50 nucleotides, at least 75 nucleotides, at least 100 nucleotides, at least 150 nucleotides, at least 200 nucleotides, at least 250 nucleotides, at least 300 nucleotides, at least 350 nucleotides, at least 400 nucleotides, at least 450 nucleotides, at least 500 nucleotides, at least 550 nucleotides, at least 600 nucleotides, at least 650 nucleotides, at least 700 nucleotides, at least 750 nucleotides, at least 800 nucleotides, at least 850 nucleotides, at least 900 nucleotides, at least 950 nucleotides, at least 1000 nucleotides, at least 1100 nucleotides, at least 1200 nucleotides, at least 1300 nucleotides, at least 1400 nucleotides, at least 1500 nucleotides, at least 1600 nucleotides, at least 1700 nucleotides, at least 1800 nucleotides, at least 2000 nucleotides, at least 2500 nucleotides, at least 3000 nucleotides, at least 3000 nucleotides, at least 4000 nucleotides, at least 5000 nucleotides, at least 6000 nucleotides, at least 7000 nucleotides, at least 8000 nucleotides, at least 9000 nucleotides, at least 10,000 nucleotides, at least 11,000 nucleotides, at least 12,000 nucleotides, at least 13,000 nucleotides, at least 14,000 nucleotides, at least 15,000 nucleotides, at least 16,000 nucleotides, at least 17,000 nucleotides, at least 18,000 nucleotides, at least 19,000 nucleotides, at least 20,000 nucleotides, at least 21,000 nucleotides, at least 22,000 nucleotides, at least 23,000 nucleotides, at least 24,000 nucleotides, or at least 25,000 nucleotides.

In some embodiments, an oligonucleotide comprising a payload sequence is between 50 and 25,000 nucleotides in length, between 100 and 20,000 nucleotides in length, between 500 and 10,000 nucleotides in length, between 1,000 and 8,000 nucleotides in length, and/or between 2,000 and 5,000 nucleotides in length.

In some embodiments, an oligonucleotide comprising a payload sequence is part of a vector.

In some embodiments, a payload oligonucleotide comprises a payload sequence and one or more sequences that encode at least one inhibitor of a NF-κB pathway and/or at least one inhibitor of an IRF pathway. In some such embodiments, a payload oligonucleotide comprises at least one internal ribosomal entry site (IRES) between a payload sequence and one or more sequences that encode at least one inhibitor of a NF-κB pathway and/or at least one inhibitor of an IRF pathway. In some embodiments, a payload sequence, a sequence encoding an inhibitor of a NF-kB pathway, a sequence encoding an inhibitor of an IRF pathway, or a combination thereof can be under the control of an IRES. In some embodiments, a payload oligonucleotide can include the following component sequences in the direction from 5′ to 3′: a cap-UTR-payload-IRES-inhibitor-UTR-polyadenyl sequence (pA), wherein the component “inhibitor” refers to one or more sequences encoding an inhibitor of a NF-kB pathway and/or an inhibitor of an IRF pathway. In some embodiments, a payload oligonucleotide can include the following component sequences in the direction from 5′ to 3′: a cap-UTR-inhibitor-IRES-payload-UTR-pA, wherein the component “inhibitor” refers to one or more sequences encoding an inhibitor of a NF-kB pathway and/or an inhibitor of an IRF pathway. In some embodiments, a payload oligonucleotide can include the following component sequences in the direction from 5′ to 3′: IRES-payload-IRES-inhibitor-UTR-pA, wherein the component “inhibitor” refers to one or more sequences encoding an inhibitor of a NF-kB pathway and/or an inhibitor of an IRF pathway. In some embodiments, a payload oligonucleotide can include the following component sequences in the direction from 5′ to 3′: IRES-inhibitor-IRES-payload-UTR-pA, wherein the component “inhibitor” refers to one or more sequences encoding an inhibitor of a NF-kB pathway and/or an inhibitor of an IRF pathway.

Inhibitors of NF-κB Pathway and Compositions that Deliver the Same

The present disclosure, among other things, provides the insight that inhibition of NF-κB signaling may reduce immunogenicity associated with RNA oligonucleotide delivery. A number of different inhibitors of a NF-κB pathway are known in the art, for example, in Gilmore & Herscovitch (2006) Oncogene, 25:6887-6899, the contents of which are incorporated herein by reference in their entirety for purposes described herein. In some embodiments, an inhibitor of a NF-κB pathway in accordance with the present disclosure is or comprises a polypeptide or a nucleic acid encoding a polypeptide.

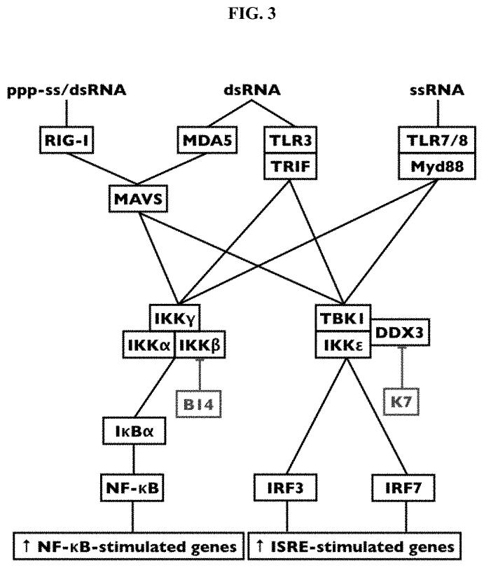

FIG. 3 is a schematic representation of an exemplary signaling network pathway showing certain elements that are involved in activation of innate immune sensors (e.g., TLR3, TLR7, TLR8, MDA5, RIG-I, etc.) by RNA (e.g., double-stranded RNA and/or single-stranded RNA) and downstream amplification cascades, for example, leading to activation of transcription of NF-κB-stimulated genes and interferon-stimulated genes. As depicted in FIG. 3, in some embodiments, NF-κB activation is mediated through IκB kinase (IKK) complex comprising an IKKα subunit, an IKKβ subunit, and an IKKγ subunit.

In some embodiments, an inhibitor of a NF-κB pathway is or comprises an agent (e.g., a polypeptide or nucleic acid agent) that inhibits activity of IKK complex. In some embodiments, an inhibitor of NF-κB signaling is or comprises an inhibitor of one or more components of IKK complex. In some embodiments, an inhibitor of a NF-κB pathway is or comprises an agent (e.g., a polypeptide or nucleic acid agent) that inhibits formation of IKK complex. In some embodiments, an inhibitor of a NF-κB pathway is or comprises an agent (e.g., a polypeptide or nucleic acid agent) that inhibits activity and/or formation of IKK complex. In some embodiments, an inhibitor of a NF-κB pathway is or comprises an agent (e.g., a polypeptide or nucleic acid agent) that binds to and/or inhibits activity and/or inhibits interaction of at least one of an IKKα subunit, an IKKβ subunit and/or an IKKγ subunit. In some embodiments, an inhibitor of a NF-κB pathway is or comprises an agent (e.g., a polypeptide or nucleic acid agent) that binds to at least one of an IKKα subunit, an IKKβ subunit and/or an IKKγ subunit. In some embodiments, an inhibitor of a NF-κB pathway is or comprises an agent (e.g., a polypeptide or nucleic acid agent) that inhibits activity of at least one of an IKKα subunit, an IKKβ subunit and/or an IKKγ subunit. In some embodiments, an inhibitor of a NF-κB pathway is or comprises an agent (e.g., a polypeptide or nucleic acid agent) that inhibits interaction of at least one of an IKKα subunit, an IKKβ subunit and/or an IKKγ subunit.

In some embodiments, an inhibitor of a NF-κB pathway is delivered as a polypeptide agent or as a nucleic acid agent. Accordingly, in some embodiments, a provided composition that delivers an inhibitor of a NF-κB pathway is or comprises a nucleic acid encoding a polypeptide agent that inhibits activity and/or formation of IKK complex. In some embodiments, a composition that delivers an inhibitor of a NF-κB pathway is or comprises a polypeptide agent that inhibits activity and/or formation of IKK complex. In some embodiments, such a polypeptide agent that inhibits activity and/or formation of IKK complex binds to and/or inhibits activity and/or inhibits interaction of at least one of an IKKα subunit, an IKKβ subunit and/or an IKKγ subunit.

In some embodiments, a composition that delivers an inhibitor of a NF-κB pathway is or comprises a nucleic acid encoding a NF-κB pathway inhibitor. In some embodiments, a nucleic acid encoding a NF-κB pathway inhibitor is or comprises a DNA oligonucleotide that encodes a NF-κB pathway inhibitor. In some embodiments, a nucleic acid encoding a NF-κB pathway inhibitor is or comprises an RNA oligonucleotide that encodes a NF-κB pathway inhibitor. In some embodiments, a RNA oligonucleotide encoding a NF-κB pathway inhibitor is or comprises a mRNA oligonucleotide. In some embodiments, a RNA oligonucleotide that encodes an inhibitor of a NF-κB pathway is or comprises a regulatory RNA (e.g., siRNA, microRNA, etc.). In some embodiments, a nucleic acid encoding a NF-κB pathway inhibitor is or comprises a vector (e.g., a DNA vector or an RNA vector). In some embodiments, a nucleic acid (e.g., a DNA oligonucleotide or an RNA oligonucleotide such as e.g., mRNA) encoding a NF-κB pathway inhibitor encodes a polypeptide that inhibits activity and/or formation of IKK complex and/or binds to and/or inhibits activity and/or interaction of at least one of an IKKα subunit, an IKKβ subunit and/or an IKKγ subunit. In some embodiments, a nucleic acid encoding a NF-κB pathway inhibitor is a regulatory RNA (e.g., siRNA, microRNA, etc.) that reduces expression of least one of an IKKα subunit, an IKKβ subunit and/or an IKKγ subunit. In some embodiments, a nucleic acid encoding a polypeptide that inhibits activity and/or formation of IKK complex and/or binds to and/or inhibits activity and/or interaction of at least one of an IKKα subunit, an IKKβ subunit and/or an IKKγ subunit is or comprises a vector (e.g., a DNA vector or an RNA vector).

In some embodiments, an inhibitor of a NF-κB pathway is or comprises an inhibitor of an IKKβ subunit. An IKKβ subunit is a component of an IKK complex, which, when activated, phosphorylates the IKBα protein, which is a regulator of the transcription factor NF-κB. Without wishing to be bound by theory, IKBα can inhibit NF-κB, for example, by masking the nuclear localization signals (NLS) of NF-κB dimes and/or by blocking the ability of NF-κB transcription factors to bind to DNA. However, following cell stimulation by extracellular stimuli, e.g., introduction of double stranded RNA and/or single-stranded RNA, which may lead to formation of an IKK complex, IKBα is phosphorylated through a cascade of inducible protein kinases that involve IKK complex, ubiquitinated, and/or degraded. This IKBα activation can lead to it dissociating from NF-κB and unmasking NLS of the NF-κB dimers, which are then are able to translocate to the nucleus and stimulate transcription of NF-κB-dependent or -driven genes including, e.g., anti-viral genes and/or pro-inflammatory genes. See, e.g., Vancurova and Vancura, “Regulation and function of nuclear IKBα in inflammation and cancer” Am J. Clin. Exp. Immunol (2012) 1: 56-66 (the contents of which are incorporated herein by reference in their entirety for purposes described herein) for additional information regarding IKBα function.

In some embodiments, a composition that delivers an inhibitor of a NF-κB pathway is or comprises a nucleic acid encoding a polypeptide agent that inhibits IKKβ. In some embodiments, a composition that delivers an inhibitor of a NF-κB pathway is or comprises a polypeptide agent that inhibits IKKβ.

In some embodiments, an inhibitor of a NF-κB pathway is or comprises a viral polypeptide or a nucleic acid encoding such a viral polypeptide. In some embodiments, an inhibitor of a NF-κB pathway is or comprises a viral innate immune repressor polypeptide or a nucleic acid encoding such a polypeptide. In some embodiments, an inhibitor of a NF-κB pathway can be obtained or derived from dsRNA viruses (e.g., Adenoviruses, Herpesviruses, Poxviruses), ssDNA viruses (e.g., Parvoviruses), dsRNA viruses (e.g., Reoviruses), (+)ssRNA viruses (single-stranded positive-sense RNA viruses, e.g., Picornaviruses, Togaviruses), (−)ssRNA viruses (single-stranded negative-antisense RNA viruses, e.g., Orthomyxoviruses, Rhabdoviruses), ssRNA-RT viruses (single-stranded positive-sense RNA viruses with reverse transcriptase (RT) and/or DNA intermediates in life-cycle (e.g., Retroviruses), dsDNA-RT viruses (double-stranded reverse transcribing viruses with RNA intermediates in life-cycle, e.g., Hepadnaviruses.

In some embodiments, an inhibitor of a NF-κB pathway is delivered as a polypeptide agent. For example, in some embodiments, a composition that delivers at least one inhibitor of a NF-κB pathway is or comprises a polypeptide agent (e.g., an innate immune repressor polypeptide agent) derived or obtained from a dsRNA virus. For example, in some embodiments, a composition that delivers an inhibitor of a NF-κB pathway is or comprises a polypeptide agent (e.g., an innate immune repressor polypeptide agent) derived or obtained from a poxvirus. In some embodiments, a composition that delivers an inhibitor of a NF-κB pathway is or comprises a vaccinia virus polypeptide agent (e.g., a vaccinia virus innate immune repressor polypeptide agent). In some embodiments, a composition that delivers an inhibitor of a NF-κB pathway is or comprises a polypeptide agent (e.g., an innate immune repressor polypeptide agent) derived from a vaccinia virus, cowpox virus, and/or variola virus.

In some embodiments, an inhibitor of a NF-κB pathway is delivered as a nucleic acid agent. For example, in some embodiments, a composition that delivers an inhibitor of a NF-κB pathway is or comprises a nucleic acid encoding a polypeptide agent (e.g., an innate immune repressor polypeptide agent) derived or obtained from a dsRNA virus. In some embodiments, a composition that delivers at least one inhibitor of a NF-κB pathway is or comprises a nucleic acid encoding a polypeptide agent (e.g., an innate immune repressor polypeptide agent) derived or obtained from a poxvirus. In some embodiments, a composition that delivers an inhibitor of a NF-κB pathway is or comprises a nucleic acid encoding a vaccinia virus polypeptide agent (e.g., a vaccinia virus innate immune repressor polypeptide agent). In some embodiments, a composition that delivers an inhibitor of a NF-κB pathway is or comprises a nucleic acid encoding a NF-κB inhibitor, where said nucleic acid is derived or obtained from a vaccinia virus, cowpox virus, and/or variola virus. In some embodiments, a composition that delivers an inhibitor of a NF-κB pathway is or comprises a nucleic acid encoding NF-κB inhibitor, where said nucleic acid is derived or obtained from a vaccinia virus.

In some embodiments, a provided composition that delivers at least one inhibitor of a NF-κB pathway includes an oligonucleotide that is derived or obtained from one or more dsRNA viruses, which oligonucleotide can function as an inhibitor of a NF-κB pathway. For example, in some embodiments, such an oligonucleotide may be or comprise an oligonucleotide sequence from a dsRNA virus (e.g., vaccinia virus).

In some embodiments, an oligonucleotide (e.g., RNA oligonucleotide) encoding a polypeptide agent (e.g., an innate immune repressor polypeptide agent) derived or obtained from a virus (e.g., a dsRNA virus) also comprises one or more UTR sequences (e.g., 3′ UTR and/or 5′ UTR sequences).

As described in Example 1 below, cells expressing Vaccinia Virus Protein B14 show a reduced level of NF-κB activation, showing that a B14 polypeptide and a nucleic acid (e.g., an RNA oligonucleotide) encoding a B14 polypeptide can function as non-limiting exemplary inhibitors of NF-κB pathway. Mechanistic studies suggest that B14 polypeptide directly binds and inhibits IKKβ, as depicted in FIG. 3. (Tang et al. (2018) “Mechanism of vaccinia viral protein B14 mediated inhibition of IκB kinase β activation,” J. Biol. Chem. 293: 10344-10352, the contents of which are incorporated by reference in their entirety for purposes described herein).

Accordingly, in some embodiments, an inhibitor of NF-κB pathway is or comprises B14 viral protein or a nucleic acid encoding the same. In some certain embodiments, an inhibitor of NF-κB pathway is or comprises a B14 polypeptide from vaccinia virus or a nucleic acid encoding the same.

In some embodiments, an inhibitor of NF-κB pathway is or comprises a B14 polypeptide that includes an amino acid sequence that is at least about 50% (e.g., at least about 55%, 60%, 65%, 70%, 75%, 80%, 85%, 90%, 95%, 96%, 97%, 98%, 99%, or higher, including and up to 100%) identical to a wild-type vaccinia virus B14 polypeptide, or a nucleic acid encoding the same. In some certain embodiments, an exemplary wild-type vaccinia virus B14 polypeptide is set forth in SEQ ID NO: 5. In some embodiments, an inhibitor of NF-κB pathway is or comprises a B14 polypeptide that includes an amino acid sequence that is at least about 80%, 85%, 90%, 95%, 96%, 97%, 98%, or 99% identical to a wild-type vaccinia virus B14 polypeptide (e.g., as set forth in SEQ ID NO: 5), or a nucleic acid encoding the same. In some embodiments, an inhibitor of NF-κB pathway is or comprises a B14 polypeptide that comprises the amino acid sequence of SEQ ID NO: 5, or a nucleic acid encoding the same. In some embodiments, an inhibitor of NF-κB pathway is a homolog or ortholog of a B14 polypeptide (e.g., ones described herein). In some embodiments, an inhibitor of NF-κB pathway is or comprises a functional domain of a B14 polypeptide that inhibits NF-κB pathway, for example, a functional domain of a B14 polypeptide that inhibits activity and/or formation of IKK complex and/or binds to and/or inhibits activity and/or interaction of at least one of an IKKα subunit, an IKKβ subunit and/or an IKKγ subunit.

In some embodiments, an inhibitor of NF-κB pathway is or comprises a nucleic acid encoding a B14 polypeptide that includes an amino acid sequence that is at least about 50% (e.g., at least about 55%, 60%, 65%, 70%, 75%, 80%, 85%, 90%, 95%, 96%, 97%, 98%, 99%, or higher, including and up to 100%) identical to a wild-type vaccinia virus B14 polypeptide (e.g., as set forth in SEQ ID NO: 5). In some embodiments, an inhibitor of NF-κB pathway is or comprises a nucleic acid encoding a B14 polypeptide that includes an amino acid sequence that is at least about 80% (e.g., at least about 85%, 90%, 95%, 96%, 97%, 98%, 99%, or higher, including and up to 100%) identical to a wild-type vaccinia virus B14 polypeptide (e.g., as set forth in SEQ ID NO: 5). In some embodiments, an inhibitor of NF-κB pathway is or comprises a nucleic acid encoding a homolog or ortholog of a B14 polypeptide (e.g., ones described herein). In some embodiments, an inhibitor of NF-κB pathway is or comprises a nucleic acid encoding a functional domain of a B14 polypeptide that inhibits activity and/or formation of IKK complex and/or binds to and/or inhibits activity and/or interaction of at least one of an IKKα subunit, an IKKβ subunit and/or an IKKγ subunit. In some embodiments, an inhibitor of NF-κB pathway is or comprise an oligonucleotide encoding a B14 polypeptide, wherein the oligonucleotide comprises a sequence that is at least about 50% (e.g., at least about 55%, 60%, 65%, 70%, 75%, 80%, 85%, 90%, 95%, 96%, 97%, 98%, 99%, or higher, including and up to 100%) identical to SEQ ID NO: 4. In some embodiments, an inhibitor of NF-κB pathway is or comprises an oligonucleotide encoding a B14 polypeptide, wherein the oligonucleotide comprises a sequence that is at least about 80%, 85%, 90%, 95%, 96%, 97%, 98%, or 99% identical to SEQ ID NO: 4. In some embodiments, an oligonucleotide encoding a B14 polypeptide comprises the sequence of SEQ ID NO: 4.

In some embodiments, an oligonucleotide encoding a B14 polypeptide also includes UTR sequences (e.g., 3′ UTR and/or 5′ UTR sequences). In some embodiments, an oligonucleotide encoding a B14 polypeptide comprises a sequence that is at least about 50% (e.g., at least about 55%, 60%, 65%, 70%, 75%, 80%, 85%, 90%, 95%, 96%, 97%, 98%, 99%, or higher, including and up to 100%) identical to SEQ ID NO: 3. In some embodiments, an oligonucleotide encoding a B14 polypeptide comprises a sequence that is at least about 80%, 85%, 90%, 95%, 96%, 97%, 98%, or 99%) identical to SEQ ID NO: 3. In some embodiments, an oligonucleotide encoding a B14 polypeptide comprises the sequence of SEQ ID NO: 3.

In some embodiments, an inhibitor of a NF-κB inhibitor pathway is or includes a viral polypeptide (e.g., a viral innate immune repressor polypeptide agent) that is from an adenovirus, a hepatitis C virus, a papilloma virus, a cytomegalovirus, and/or a Mollusum contagiosum virus. In some embodiments, an inhibitor of a NF-κB pathway is or comprises a nucleic acid encoding NF-κB inhibitor polypeptide or a viral innate immune repressor polypeptide, where said nucleic acid is derived from an adenovirus, a hepatitis C virus, a papilloma virus, a cytomegalovirus, and/or a Mollusum contagiosum virus. For example, in some embodiments, an inhibitor of a NF-κB inhibitor pathway is or comprises a viral polypeptide derived or obtained from an adenovirus E3-14.7K, an adenovirus E3-10.4K/14.5K, a hepatitis C core protein, a papillomavirus E7, and/or a Mollusum contagiosum MC160, or a nucleic acid encoding the same.

In some embodiments, an inhibitor of a NF-κB pathway is from a non-viral polypeptide, or a nucleic acid encoding such a non-viral polypeptide. For example, in some embodiments, such an inhibitor of a NF-κB pathway may be a dominant-negative form of a mammalian (e.g., human) gene that is associated or interacts with a NF-κB pathway (e.g., as illustrated in FIG. 3), or a nucleic acid encoding such a dominant-negative form. In some embodiments, such an inhibitor of a NF-κB pathway may be or comprise a phosphorylation defective mutant of I kappa B alpha, or a nucleic acid encoding the same. In some embodiments, an inhibitor of a NF-κB pathway may be or comprise an exemplary phosphorylation defective I kappa B alpha that blocks NF-κB activity, e.g., as described in Fujioka et al. Oncogene (2003) 22(9): 1365-70, the contents of which are incorporated herein by reference in their entirety for purposes described herein, or a nucleic acid encoding the same. In some embodiments, an inhibitor of a NF-κB pathway may be or comprise a kinase defective mutant of IkappaB kinase (IKK)-1 and/or IKK-2, or a nucleic acid encoding the same. In some embodiments, an inhibitor of a NF-κB pathway may be or comprise an exemplary kinase defective IKK-1 and/or kinase defective IKK-2 that blocks NF-κB activity, e.g., as described in Mercurio et al. Science (1997) 278(5339): 860-6, the contents of which are incorporated herein by reference in their entirety for purposes described herein, or a nucleic acid encoding the same.

In some embodiments, a composition that delivers one or more inhibitors of a NF-κB pathway includes one or more nucleic acids (e.g., an RNA oligonucleotide such as, e.g., mRNA) encoding at least one or more (including, e.g., at least 2, at least 3, at least 4, at least 5, at least 6, at least 7, at least 8, at least 9, at least 10, or more) inhibitors of NF-κB pathway. In some certain embodiments where a composition delivers a plurality of (e.g., at least 2, at least 3, at least 4, at least 5, at least 6, at least 7, at least 8, at least 9, at least 10, or more) inhibitors of NF-κB pathway, such a plurality of inhibitors may be encoded in the same nucleic acid construct or encoded in two or more different nucleic acid constructs.

In some embodiments, a nucleic acid (e.g., an RNA oligonucleotide such as, e.g., mRNA) encoding at least one or more (including, e.g., at least 2, at least 3, at least 4, at least 5, at least 6, at least 7, at least 8, at least 9, at least 10, or more) inhibitors of NF-κB pathway can also comprise a sequence that encodes an innate immune repressor polypeptide that is associated with a different pathway, e.g., but not limited to an IRF pathway as described herein.

Inhibitors of IRF Pathway and Compositions that Deliver the Same

The present disclosure provides the insight that inhibition of IRF signaling may reduce immunogenicity associated with RNA oligonucleotide delivery. Interferon signaling is believed to be activated in response to viral infection (e.g., through recognition of viral DNA).

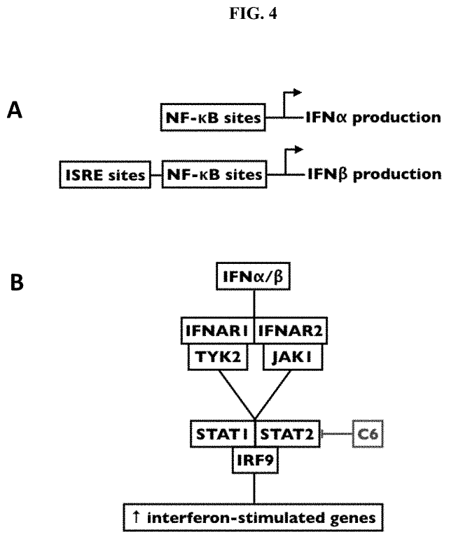

As depicted in FIG. 3, recognition of viral nucleic acids (e.g., double-stranded and/or single-stranded nucleic acids) leads to activation of innate immune sensors (e.g., TLR3, TLR7, TLR8, MDA5, RIG-I, etc.), which signaling is cascaded and leads to transcription of NF-κB-stimulated genes and interferon-stimulated genes. In some embodiments, activation of transcription of interferon-stimulated target genes is mediated through a complex of IKK-related kinases including TANK-binding kinase 1 (TBK1) and IκB kinase ε (IKKε, also known as IKKi). As shown in FIG. 3, TBK1 and IKK-ε complex interacts with DDX3, a DEAD-box ATP-dependent-RNA-helicase. Transcription of interferon-stimulated genes are also activated through canonical interferon signaling pathways through activation of Janus kinase (JAK)-signal transducer and activator of transcription (STAT) pathway. A schematic of an exemplary canonical interferon signaling pathway is depicted in FIG. 4, panel B.

In some embodiments, an inhibitor of an IRF pathway is or comprises an agent (e.g., a polypeptide or nucleic acid agent) that inhibits activity and/or formation of an IKK-related kinase complex and/or JAK-STAT signaling.

In some embodiments, an inhibitor of an IRF pathway is delivered as a polypeptide agent or as a nucleic acid agent. In some embodiments, a composition that delivers an IRF pathway inhibitor is or comprises a nucleic acid encoding an IRF pathway inhibitor. In some embodiments, a nucleic acid encoding an IRF pathway inhibitor is a DNA oligonucleotide that encodes an IRF pathway inhibitor. In some embodiments, a nucleic acid encoding an IRF pathway inhibitor is a RNA oligonucleotide that encodes an IRF pathway inhibitor. In some embodiments, a RNA oligonucleotide encoding an IRF pathway inhibitor is a mRNA oligonucleotide. In some embodiments, a RNA oligonucleotide that encodes an IRF pathway inhibitor is a regulatory RNA (e.g., siRNA, microRNA, etc.). In some embodiments, a nucleic acid encoding an IRF pathway inhibitor is or comprises a vector (e.g., a DNA vector or an RNA vector). In some embodiments, a nucleic acid (e.g., a DNA oligonucleotide or an RNA oligonucleotide such as e.g., mRNA) encoding an IRF pathway inhibitor encodes a polypeptide that inhibits activity and/or formation of an IKK-related kinase complex (comprising TBK1, DDX3, and/or IKKε) and/or JAK-STAT signaling. In some embodiments, nucleic acid encoding an IRF pathway inhibitor is a regulatory RNA (e.g., siRNA, microRNA, etc.) that reduces expression of least one component and/or activity of an IKK-related kinase complex (comprising TBK1, DDX3, and/or IKKε) and/or JAK-STAT signaling. In some embodiments, a nucleic acid (e.g., a DNA oligonucleotide or an RNA oligonucleotide such as e.g., mRNA) encoding a polypeptide that inhibits activity and/or formation of an IKK-related kinase complex (comprising TBK1, DDX3, and/or IKKε) and/or JAK-STAT signaling is or comprises a vector (e.g., a DNA vector or an RNA vector).

As described in Example 2 below, cells expressing Vaccinia Virus Protein K7 show a reduced level of IRF activation, showing that a K7 polypeptide and a nucleic acid (e.g., an RNA oligonucleotide) encoding a K7 polypeptide can function as non-limiting exemplary inhibitors of IRF pathway. Without wishing to be limited by theory, K7 is thought to inhibit DEAD box protein 3 (DDX3) and thereby prevent TBK1/IKKε activation of IRF-3 and IRF-7 (Schroder et al., (2008) EMBO J., 27(15):2147-57, the contents of which are incorporated by reference in their entirety for purposes described herein).

In some embodiments, an inhibitor of an IRF pathway is or comprises an agent (e.g., a polypeptide agent or a nucleic acid agent) that inhibits activity of DDX3, TBK1, and/or IKKε. In some embodiments, an inhibitor of a IRF pathway is or comprises an agent (e.g., a polypeptide agent or a nucleic acid agent) that inhibits association of DDX3, TBK1, and/or IKKε

In some embodiments, an inhibitor of an IRF pathway is or comprises a an agent (e.g., a polypeptide agent or a nucleic acid agent) that inhibits activity of a IKK-related kinase complex (including, e.g., TBK1 and IKKε). In some embodiments, an inhibitor of an IRF pathway is or comprises an agent (e.g., a polypeptide agent or a nucleic acid agent) that inhibits activity and/or formation of a complex comprising TBK1 and IKKε. In some embodiments, an inhibitor of an IRF pathway is or comprises an agent (e.g., a polypeptide agent or a nucleic acid agent) that binds to TBK1 and/or IKKε. In some embodiments, an inhibitor of an IRF pathway is or comprises an agent (e.g., a polypeptide agent or a nucleic acid agent) that inhibits activity or association and/or interaction of a TBK1/IKKε complex.

In some embodiments, an inhibitor of an IRF pathway is or comprises an agent (e.g., a polypeptide agent or a nucleic acid agent) that binds to DEAD box protein 3 (DDX3). In some embodiments, an inhibitor of an IRF pathway is or comprises an agent (e.g., a polypeptide agent or a nucleic acid agent) that inhibits activity of DDX3 and/or interaction of DDX3 with a TBK1/IKKε complex.

In some embodiments, a composition that delivers an inhibitor of an IRF pathway is or comprises a nucleic acid encoding a polypeptide agent that inhibits activity of DDX3 and/or association of DDX3 with a TBK1/IKKε complex. In some embodiments, a composition that delivers an inhibitor of an IRF pathway is or comprises a polypeptide agent that inhibits DDX3 and/or association of DDX3 with a TBK1/IKKε complex.

Example 2 below further describes identifying that cells expressing Vaccinia Virus Protein C6 show a reduced level of IRF activation, showing that a C6 polypeptide and a nucleic acid (e.g., an RNA oligonucleotide) encoding a C6 polypeptide can function as non-limiting exemplary inhibitors of IRF pathway. Without wishing to be limited by theory, C6 is thought to bind to STAT2 (Stuart et al., (2016) PLOS Pathogens 12(12): e1005955, the contents of which are incorporated by reference in their entirety for purposes described herein), which may allow it to inhibit interferon-dependent activation of JAK/STAT signaling. As depicted in FIG. 4, STAT-2 forms a complex with STAT-1 and IRF-9.

In some embodiments, an inhibitor of an IRF pathway is or comprises an agent (e.g., a polypeptide agent or a nucleic acid agent) that inhibits JAK-STAT signaling. In some embodiments, an inhibitor of an IRF pathway is or comprises an agent (e.g., a polypeptide agent or a nucleic acid agent) that inhibits activity of STAT1, STAT2, and/or IRF9. In some embodiments, an inhibitor of an IRF pathway is or comprises an agent (e.g., a polypeptide agent or a nucleic acid agent) that inhibits association and/or interaction of STAT1, STAT2, and/or IRF9. In some embodiments, an inhibitor of an IRF pathway is or comprises an agent (e.g., a polypeptide agent or a nucleic acid agent) that inhibits activity and/or formation of a complex comprising STAT1, STAT2, and IRF9. In some embodiments, an inhibitor of an IRF pathway is or comprises an agent (e.g., a polypeptide agent or a nucleic acid agent) that binds to STAT1, STAT2, and/or IRF9.

In some embodiments, an inhibitor of an IRF pathway is or comprises an agent (e.g., a polypeptide agent or a nucleic acid agent) that binds to STAT2. In some embodiments, an inhibitor of an IRF pathway is or comprises (e.g., a polypeptide agent or a nucleic acid agent) that inhibits activity of STAT2.

In some embodiments, an inhibitor of IRF pathway is or comprises a viral polypeptide or a nucleic acid encoding such a viral polypeptide. In some embodiments, an inhibitor of IRF pathway is or comprises a viral innate immune repressor polypeptide or a nucleic acid encoding such a polypeptide. In some embodiments, an inhibitor of IRF pathway can be obtained or derived from dsRNA viruses (e.g., Vacciniaviruses, Adenoviruses, Herpesviruses, Poxviruses), ssDNA viruses (e.g., Parvoviruses), dsRNA viruses (e.g., Reoviruses), (+)ssRNA viruses (single-stranded positive-sense RNA viruses, e.g., Picornaviruses, Togaviruses), (−)ssRNA viruses (single-stranded negative-antisense RNA viruses, e.g., Orthomyxoviruses, Rhabdoviruses), ssRNA-RT viruses (single-stranded positive-sense RNA viruses with reverse transcriptase (RT) and/or DNA intermediates in life-cycle (e.g., Retroviruses), dsDNA-RT viruses (double-stranded reverse transcribing viruses with RNA intermediates in life-cycle, e.g., Hepadnaviruses.

In some embodiments, an inhibitor of an IRF pathway is delivered as a polypeptide agent. For example, in some embodiments, a composition that delivers at least one inhibitor of an IRF pathway is or comprises a polypeptide agent (e.g., an innate immune repressor polypeptide agent) derived or obtained from a dsRNA virus. For example, in some embodiments, a composition that delivers an inhibitor of an IRF pathway is or comprises a polypeptide agent (e.g., an innate immune repressor polypeptide agent) derived or obtained from a poxvirus. For example, in some embodiments, a composition that delivers at least one inhibitor of an IRF pathway is or comprises a vaccinia virus polypeptide agent (e.g., a vaccinia virus innate immune repressor polypeptide agent). In some embodiments, a composition that delivers at least one inhibitor of an IRF pathway is or comprises a polypeptide (e.g., an innate immune repressor polypeptide agent) derived from a vaccinia virus, cowpox virus, and/or variola virus.

In some embodiments, an inhibitor of an IRF pathway is delivered as a nucleic acid agent. For example, in some embodiments, a composition that delivers an inhibitor of an IRF pathway is or comprises a nucleic acid encoding a polypeptide agent (e.g., an innate immune repressor polypeptide agent) derived or obtained from a dsRNA virus. In some embodiments, a composition that delivers at least one inhibitor of an IRF pathway is or comprises a nucleic acid encoding a polypeptide agent (e.g., an innate immune repressor polypeptide agent) derived or obtained from a poxvirus. In some embodiments, a composition that delivers an inhibitor of an IRF pathway is or comprises a nucleic acid encoding a vaccinia virus polypeptide agent (e.g., a vaccinia virus innate immune repressor polypeptide agent). In some embodiments, a composition that delivers an inhibitor of an IRF pathway is or comprises a nucleic acid encoding IRF inhibitor, where said nucleic acid is derived or obtained from a vaccinia virus, cowpox virus, and/or variola virus. In some embodiments, a composition that delivers an inhibitor of an IRF pathway is or comprises a nucleic acid encoding IRF inhibitor, where said nucleic acid is derived or obtained from a vaccinia virus.

In some embodiments, a provided composition that delivers at least one inhibitor of an IRF pathway includes an oligonucleotide that is derived or obtained from one or more dsRNA viruses, which oligonucleotide can function as an inhibitor of an IRF pathway. For example, in some embodiments, such an oligonucleotide may be or comprise an oligonucleotide sequence from a dsRNA virus (e.g., vaccinia virus).

In some embodiments, an oligonucleotide (e.g., RNA oligonucleotide) encoding a polypeptide agent (e.g., an innate immune repressor polypeptide agent) derived or obtained from a virus (e.g., a dsRNA virus) also comprises one or more UTR sequences (e.g., 3′ UTR and/or 5′ UTR sequences).

In some embodiments, an inhibitor of IRF pathway is or comprises viral protein K7 or a nucleic acid encoding the same. In some certain embodiments, an inhibitor of IRF pathway is or comprises a K7 polypeptide from vaccinia virus or a nucleic acid encoding the same.

In some embodiments, an inhibitor of IRF pathway is or comprises a K7 polypeptide that includes an amino acid sequence that is at least about 50% (e.g., at least about 55%, 60%, 65%, 70%, 75%, 80%, 85%, 90%, 95%, 96%, 97%, 98%, 99%, or higher, including and up to 100%) identical to a wild-type vaccinia virus K7 polypeptide, or a nucleic acid encoding the same. In some certain embodiments, an exemplary wild-type vaccinia virus K7 polypeptide is set forth in SEQ ID NO: 9. In some embodiments, an inhibitor of IRF pathway is or comprises a K7 polypeptide that includes an amino acid sequence that is at least about 80%, 85%, 90%, 95%, 96%, 97%, 98%, or 99% identical to a wild-type vaccinia virus K7 polypeptide (e.g., as set forth in SEQ ID NO: 9), or a nucleic acid encoding the same. In some embodiments, an inhibitor of IRF pathway is or comprises a K7 polypeptide that comprises the amino acid sequence of SEQ ID NO: 9, or a nucleic acid encoding the same. In some embodiments, an inhibitor of IRF pathway is or comprises a homolog or ortholog of a K7 polypeptide (e.g., ones described herein). In some embodiments, an inhibitor of IRF pathway is or comprises a functional domain of a K7 polypeptide that inhibits IRF pathway, e.g., a functional domain of a K7 polypeptide (e.g., ones described herein) that inhibits activity or association and/or interaction of a TBK1/IKKε complex and/or a functional domain of a K7 polypeptides (e.g., ones described herein) that binds to DDX3 and/or inhibits activity of DDX3 and/or interaction of DDX3 with a TBK1/IKKε complex.

In some embodiments, an inhibitor of IRF pathway is or comprises a nucleic acid encoding a K7 polypeptide that includes an amino acid sequence that is at least about 50% (e.g., at least about 55%, 60%, 65%, 70%, 75%, 80%, 85%, 90%, 95%, 96%, 97%, 98%, 99%, or higher, including and up to 100%) identical to a wild-type vaccinia virus K7 polypeptide (e.g., as set forth in SEQ ID NO: 9). In some embodiments, an inhibitor of IRF pathway is or comprises a nucleic acid encoding a K7 polypeptide that includes an amino acid sequence that is at least about 80% (e.g., at least about 85%, 90%, 95%, 96%, 97%, 98%, 99%, or higher, including and up to 100%) identical to a wild-type vaccinia virus K7 polypeptide (e.g., as set forth in SEQ ID NO: 9). In some embodiments, an inhibitor of IRF pathway is or comprises a nucleic acid encoding a homolog or ortholog of a K7 polypeptide (e.g., ones described herein). In some embodiments, an inhibitor of IRF pathway is or comprises a nucleic acid encoding a functional domain of a K7 polypeptide that inhibits IRF pathway, e.g., a functional domain of a K7 polypeptide (e.g., ones described herein) that inhibits activity or association and/or interaction of a TBK1/IKKε complex and/or a functional domain of a K7 polypeptides (e.g., ones described herein) that binds to DDX3 and/or inhibits activity of DDX3 and/or interaction of DDX3 with a TBK1/IKKε complex.

In some embodiments, an inhibitor of IRF is or comprises an oligonucleotide encoding a K7 polypeptide, wherein the oligonucleotide comprises a sequence that is at least about 50% (e.g., at least about 55%, 60%, 65%, 70%, 75%, 80%, 85%, 90%, 95%, 96%, 97%, 98%, 99%, or higher, including and up to 100%) identical to SEQ ID NO: 8. In some embodiments, an inhibitor of IRF signaling is or comprises an oligonucleotide encoding a K7 polypeptide, wherein the oligonucleotide comprises a sequence that is at least about 80%, 85%, 90%, 95%, 96%, 97%, 98%, or 99% identical to SEQ ID NO: 8. In some embodiments, an oligonucleotide encoding a K7 polypeptide comprises the sequence of SEQ ID NO: 8.

In some embodiments, an oligonucleotide encoding a K7 polypeptide also includes UTR sequences (e.g., 3′ UTR and/or 5′ UTR sequences). In some embodiments, an oligonucleotide encoding a K7 polypeptide comprises a sequence that is at least about 50% (e.g., at least about 55%, 60%, 65%, 70%, 75%, 80%, 85%, 90%, 95%, 96%, 97%, 98%, 99%, or higher, including and up to 100%) identical to SEQ ID NO: 7. In some embodiments, an oligonucleotide encoding a K7 polypeptide comprises a sequence that is at least about 80%, 85%, 90%, 95%, 96%, 97%, 98%, or 99% identical to SEQ ID NO: 7. In some embodiments, an oligonucleotide encoding a K7 polypeptide comprises the sequence of SEQ ID NO: 7.

In some embodiments, an inhibitor of IRF signaling is or comprises viral protein C6 or a nucleic acid encoding the same. In some certain embodiments, an inhibitor of IRF signaling is or comprises a C6 polypeptide from vaccinia virus or a nucleic acid encoding the same.

In some embodiments, an inhibitor of IRF pathway is or comprises a C6 polypeptide that includes an amino acid sequence that is at least about 50% (e.g., at least about 55%, 60%, 65%, 70%, 75%, 80%, 85%, 90%, 95%, 96%, 97%, 98%, 99%, or higher, including and up to 100%) identical to a wild-type vaccinia virus C6 polypeptide, or a nucleic acid encoding the same. In some certain embodiments, an exemplary wild-type vaccinia virus C6 polypeptide is set forth in SEQ ID NO: 12. In some embodiments, an inhibitor of IRF pathway is or comprises a C6 polypeptide that includes an amino acid sequence that is at least about 80%, 85%, 90%, 95%, 96%, 97%, 98%, or 99% identical to a wild-type vaccinia virus C6 polypeptide (e.g., as set forth in SEQ ID NO: 12). In some embodiments, an inhibitor of IRF pathway is or comprises a C6 polypeptide that comprises the amino acid sequence of SEQ ID NO: 12. In some embodiments, an inhibitor of IRF pathway is or comprises a homolog or ortholog of a C6 polypeptide (e.g., ones described herein). In some embodiments, an inhibitor of IRF pathway is or comprises a functional domain of a C6 polypeptide that inhibits JAK-STAT pathway, e.g., a functional domain of a C6 polypeptide (e.g., ones described herein) that inhibits activity and/or association and/or interaction of STAT1, STAT2, and/or IRF9.

In some embodiments, an inhibitor of IRF pathway is or comprises a nucleic acid encoding a C6 polypeptide that includes an amino acid sequence that is at least about 50% (e.g., at least about 55%, 60%, 65%, 70%, 75%, 80%, 85%, 90%, 95%, 96%, 97%, 98%, 99%, or higher, including and up to 100%) identical to a wild-type vaccinia virus C6 polypeptide (e.g., as set forth in SEQ ID NO: 12). In some embodiments, an inhibitor of IRF pathway is or comprises a nucleic acid encoding a C6 polypeptide that includes an amino acid sequence that is at least about 80% (e.g., at least about 85%, 90%, 95%, 96%, 97%, 98%, 99%, or higher, including and up to 100%) identical to a wild-type vaccinia virus C6 polypeptide (e.g., as set forth in SEQ ID NO: 12). In some embodiments, an inhibitor of IRF pathway is or comprises a nucleic acid encoding a homolog or ortholog of a C6 polypeptide (e.g., ones described herein). In some embodiments, an inhibitor of IRF pathway is or comprises a nucleic acid encoding a functional domain of a C6 polypeptide that inhibits JAK-STAT pathway, e.g., a functional domain of a C6 polypeptide (e.g., ones described herein) that inhibits activity and/or association and/or interaction of STAT1, STAT2, and/or IRF9.

In some embodiments, an inhibitor of IRF signaling is or comprises an oligonucleotide encoding a C6 polypeptide, wherein the oligonucleotide comprises a sequence that is at least about 50% (e.g., at least about 55%, 60%, 65%, 70%, 75%, 80%, 85%, 90%, 95%, 96%, 97%, 98%, 99%, or higher, including and up to 100%) identical to SEQ ID NO: 11. In some embodiments, an inhibitor of IRF signaling is or comprises an oligonucleotide encoding a C6 polypeptide, wherein the oligonucleotide comprises a sequence that is at least about 80%, 85%, 90%, 95%, 96%, 97%, 98%, or 99% identical to SEQ ID NO: 11. In some embodiments, an oligonucleotide encoding a C6 polypeptide comprises the sequence of SEQ ID NO: 11.

In some embodiments, an oligonucleotide encoding a C6 polypeptide also includes UTR sequences (e.g., 3′ UTR and/or 5′ UTR sequences). In some embodiments, an oligonucleotide encoding a C6 polypeptide comprises a sequence that is at least about 50% (e.g., at least about 55%, 60%, 65%, 70%, 75%, 80%, 85%, 90%, 95%, 96%, 97%, 98%, 99%, or higher, including and up to 100%) identical to SEQ ID NO: 10. In some embodiments, an oligonucleotide encoding a C6 polypeptide comprises a sequence that is at least about 80%, 85%, 90%, 95%, 96%, 97%, 98%, or 99% identical to SEQ ID NO: 10. In some embodiments, an oligonucleotide encoding a C6 polypeptide comprises the sequence of SEQ ID NO: 10.

In some embodiments, an inhibitor of an IRF pathway is from a non-viral polypeptide, or a nucleic acid encoding such a non-viral polypeptide. For example, in some embodiments, such an inhibitor of an IRF pathway may be a dominant-negative form of a mammalian (e.g., human) gene that is associated or interacts with an IRF pathway (e.g., as illustrated in FIG. 3), or a nucleic acid encoding such a dominant-negative form. In some embodiments, such an inhibitor of an IRF pathway may be or comprise a dominant negative mutant of IRF3, or a nucleic acid encoding the same. In some embodiments, an inhibitor of an IRF pathway may be or comprise an exemplary dominant negative mutant of IRF3, which lacks a portion of a DNA binding domain like IRF3a, e.g., as described in Kim et al. J. Biol. Chem (2003) 278(17): 15272-8, the contents of which are incorporated herein by reference in their entirety for purposes described herein, or a nucleic acid encoding the same. In some embodiments, an inhibitor of an IRF pathway may be or comprise a dominant negative mutant of IRF-7, or a nucleic acid encoding the same. In some embodiments, an inhibitor of an IRF pathway may be or comprise an exemplary dominant negative mutant of IRF-7, which has an amino-terminal deletion of a DNA binding domain and a transactivation domain, e.g., as described in Au et al. Virology (2001) 280 (2): 273-82, the contents of which are incorporated herein by reference in their entirety for purposes described herein, or a nucleic acid encoding the same.

In some embodiments, a composition that delivers one or more inhibitors of an IRF pathway includes one or more nucleic acids (e.g., an RNA oligonucleotide such as, e.g., mRNA) encoding at least one or more (including, e.g., at least 2, at least 3, at least 4, at least 5, at least 6, at least 7, at least 8, at least 9, at least 10, or more) inhibitors of an IRF pathway. In some certain embodiments where a composition delivers a plurality of (e.g., at least 2, at least 3, at least 4, at least 5, at least 6, at least 7, at least 8, at least 9, at least 10, or more) inhibitors of an IRF pathway, such a plurality of inhibitors may be encoded in the same nucleic acid construct or encoded in two or more different nucleic acid constructs.

In some embodiments, a nucleic acid (e.g., an RNA oligonucleotide such as, e.g., mRNA) encoding at least one or more (including, e.g., at least 2, at least 3, at least 4, at least 5, at least 6, at least 7, at least 8, at least 9, at least 10, or more) inhibitors of an IRF pathway can also comprise a sequence that encodes an innate immune repressor polypeptide that is associated with a different pathway, e.g., but not limited to a NF-κB pathway as described herein.

### II. Compositions

Other aspects of the present disclosure provides compositions comprising any component, or combination of components, of a nucleic acid expression system as described herein. In some embodiments, compositions described herein are useful for improving delivery of oligonucleotides (e.g., RNA oligonucleotides, e.g., mRNA oligonucleotides) comprising a payload sequence. In some embodiments, compositions described herein are useful for improving the effectiveness of oligonucleotide therapeutics and vaccines (e.g., based on RNA oligonucleotides). In some embodiments, compositions described herein are useful for reducing non-specific toxicity induced by oligonucleotide therapeutics and vaccines. In some embodiments, compositions described herein are useful for reducing innate immunity-triggered suppression of protein translation and/or mRNA degradation. In some embodiments, compositions described herein are useful for enhancing expression and/or activity of a payload sequence to be introduced into target cells.

In some aspects, the present disclosure provides one or more compositions that deliver at least one or more (e.g., at least two or more) inhibitors of a NF-κB pathway and/or at least one or more (e.g., at least two or more) inhibitors of an IRF pathway. In some embodiments, a composition is configured to deliver one or more inhibitors of a NF-κB pathway (e.g., ones as described herein such as, e.g., an inhibitor of one or more components of IKK complex). In some embodiments, a composition is configured to deliver one or more inhibitors of an IRF pathway (e.g., ones as described herein such as, e.g., an inhibitor of an IKK-like complex, and/or an inhibitor of a JAK-STAT pathway).

In some embodiments, a composition is configured to deliver at least one or more (e.g., 1, 2, 3, 4, 5, 6, 7, 8, 9, 10, or more) inhibitors of a NF-κB pathway and at least one or more (e.g., 1, 2, 3, 4, 5, 6, 7, 8, 9, 10, or more) inhibitors of an IRF pathway. In some embodiments, a composition comprises at least one or more polypeptide inhibitors of a NF-κB pathway and at least one or more polypeptide inhibitors of an IRF pathway. In some embodiments, a composition comprises at least one or more oligonucleotide inhibitors of a NF-κB pathway (e.g., an oligonucleotide encoding a polypeptide inhibitor) and at least one or more polypeptide inhibitors of an IRF pathway. In some embodiments, a composition comprises at least one or more polypeptide inhibitors of a NF-03 pathway and at least one or more oligonucleotide inhibitors of an IRF pathway (e.g., an oligonucleotide encoding a polypeptide inhibitor). In some embodiments, a composition comprises at least one or more oligonucleotide inhibitors of a NF-κB pathway and at least one or more oligonucleotide inhibitors of an IRF pathway (e.g., oligonucleotides encoding polypeptide inhibitors). In some certain embodiments, a composition comprises an oligonucleotide comprising a sequence that encodes at least two polypeptide inhibitors, which comprises at least one inhibitor of a NF-κB pathway and at least one inhibitor of an IRF pathway. In some certain embodiments, a composition comprises an oligonucleotide comprising a sequence that encodes at least two polypeptide inhibitors, one of which is or comprises at least one inhibitor of a NF-κB pathway. In some certain embodiments, a composition comprises an oligonucleotide comprising a sequence that encodes at least two polypeptide inhibitors, one of which is or comprises at least one inhibitor of an IRF pathway.

In some embodiments, a composition comprises an oligonucleotide encoding both an inhibitor of a NF-κB pathway and an inhibitor of an IRF pathway. In some embodiments, a composition comprises one or more oligonucleotides encoding an inhibitor of a NF-κB pathway and an inhibitor of an IRF pathway.

In some embodiments, a composition comprises a plurality of (e.g., at least 2, at least 3, at least 4, at least 5, at least 6, at least 7, at least 8, at least 9, at least 10, or more) oligonucleotides, wherein each oligonucleotide encodes at least one distinct inhibitor of a NF-κB pathway or at least one distinct inhibitor of an IRF pathway. In some certain embodiments, such a plurality of oligonucleotide may comprise an oligonucleotide, which comprises a sequence that encodes at least two innate immune repressor polypeptides, e.g., which in some embodiments may be or comprise an inhibitor of a NF-κB pathway (e.g., ones as described herein) and an inhibitor of an IRF pathway (e.g., ones as described herein).

In some embodiments, a composition is configured to deliver two or more inhibitors (e.g., as polypeptide agents and/or nucleic acid agents) selected from: (i) an inhibitor of IKK complex (e.g., an inhibitor of IKKα, IKKβ, and/or IKKγ such as ones described herein), (ii) an inhibitor of a IKK-like complex (e.g., an inhibitor of TBK1, IKKε, and/or DDX3 such as ones described herein), and (iii) an inhibitor of a JAK-STAT pathway (e.g., an inhibitor of STAT1, STAT2, and/or IRF9 such as ones described herein).

In some embodiments, a composition is configured to deliver (e.g., as polypeptide agents and/or a nucleic acid agents) at least one inhibitor of IKK complex (e.g., an inhibitor of IKKα, IKKβ, and/or IKKγ such as ones described herein) and one or more IRF pathway inhibitors selected from: (i) an inhibitor of a IKK-like complex (e.g., an inhibitor of TBK1, IKKε, and/or DDX3 such as ones described herein), and (ii) an inhibitor of a JAK-STAT pathway (e.g., an inhibitor of STAT1, STAT2, and/or IRF9 such as ones described herein).

In some embodiments, one or more compositions of the present disclosure is configured to deliver a Vaccinia Virus Protein B14 polypeptide and Vaccinia Virus Protein K7 polypeptide (e.g., as polypeptide agents and/or nucleic acid agents). In some embodiments, one or more compositions of the present disclosure deliver a Vaccinia Virus Protein B14 polypeptide and Vaccinia Virus Protein C6 polypeptide (e.g., as polypeptide agents and/or nucleic acid agents). In some embodiments, one or more compositions of the present disclosure deliver a Vaccinia Virus Protein K7 polypeptide and Vaccinia Virus Protein C6 polypeptide (e.g., as polypeptide agents and/or nucleic acid agents). In some embodiments, one or more compositions of the present disclosure deliver a Vaccinia Virus Protein B14 polypeptide, Vaccinia Virus Protein K7 polypeptide, and a Vaccinia Virus Protein C6 polypeptide (e.g., as polypeptide agents and/or nucleic acid agents).

In some aspects, the present disclosure provides a composition comprising at least one oligonucleotide comprising a payload sequence as described herein. In some embodiments, a composition comprises 1, 2, 3, 4, 5, 6, 7, 8, 9, or 10 oligonucleotides, each comprising a payload sequence.

In some embodiments, a composition comprises at least one oligonucleotide (e.g., RNA oligonucleotide such as, e.g., mRNA) comprising a payload sequence and one or more sequences that encode at least one inhibitor of a NF-κB pathway and/or at least one inhibitor of an IRF pathway. In some such embodiments, a composition comprises at least one oligonucleotide (e.g., RNA oligonucleotide such as, e.g., mRNA) comprising at least one internal ribosomal entry site (IRES) between a payload sequence and one or more sequences that encode at least one inhibitor of a NF-κB pathway and/or at least one inhibitor of an IRF pathway. In some embodiments, a payload sequence, a sequence encoding an inhibitor of a NF-kB pathway, a sequence encoding an inhibitor of an IRF pathway, or a combination thereof can be under the control of an IRES. In some embodiments, an oligonucleotide provided in a composition can include the following component sequences in the direction from 5′ to 3′: a cap-UTR-payload-IRES-inhibitor-UTR-polyadenyl sequence (pA), wherein the component “inhibitor” refers to one or more sequences encoding an inhibitor of a NF-kB pathway and/or an inhibitor of an IRF pathway. In some embodiments, an oligonucleotide provided in a composition can include the following component sequences in the direction from 5′ to 3′: a cap-UTR-inhibitor-IRES-payload-UTR-pA, wherein the component “inhibitor” refers to one or more sequences encoding an inhibitor of a NF-kB pathway and/or an inhibitor of an IRF pathway. In some embodiments, an oligonucleotide provided in a composition can include the following component sequences in the direction from 5′ to 3′: IRES-payload-IRES-inhibitor-UTR-pA, wherein the component “inhibitor” refers to one or more sequences encoding an inhibitor of a NF-kB pathway and/or an inhibitor of an IRF pathway. In some embodiments, an oligonucleotide provided in a composition can include the following component sequences in the direction from 5′ to 3′: IRES-inhibitor-IRES-payload-UTR-pA, wherein the component “inhibitor” refers to one or more sequences encoding an inhibitor of a NF-kB pathway and/or an inhibitor of an IRF pathway.

In some embodiments, a composition comprises at least one RNA oligonucleotide (e.g., mRNA oligonucleotide) comprising a payload sequence as described herein. In some embodiments, a composition comprises 1, 2, 3, 4, 5, 6, 7, 8, 9, or 10 RNA oligonucleotides, each comprising a payload sequence.

In some embodiments, a composition comprises any embodiment of a nucleic acid expression system described herein.

In some embodiments, a composition comprising a payload oligonucleotide further includes an inhibitor of a NF-κB pathway and/or an inhibitor of an IRF pathway. In some embodiments, a composition comprising a payload oligonucleotide further includes at least one inhibitor of IKK complex (e.g., an inhibitor of IKKα, IKKβ, and/or IKKγ), which in some embodiments may be a polypeptide agent or a nucleic acid agent. In some embodiments, a composition comprising a payload oligonucleotide further includes at least one IRF pathway inhibitor, which in some embodiments may be a polypeptide agent or a nucleic acid agent, selected from: (i) an inhibitor of a IKK-like complex (e.g., an inhibitor of TBK1, IKKε, and/or DDX3), and (ii) an inhibitor of a JAK-STAT pathway (e.g., an inhibitor of STAT1, STAT2, and/or IRF9). In some embodiments, a composition comprising a payload oligonucleotide further includes an inhibitor of a NF-κB pathway and an inhibitor of an IRF pathway (e.g., a polypeptide inhibitor and/or an oligonucleotide encoding an inhibitor).

In some embodiments, a composition comprises an oligonucleotide encoding an NF-κB pathway inhibitor and/or an oligonucleotide encoding an IRF pathway inhibitor. In some embodiments, a composition comprises an RNA oligonucleotide encoding an NF-κB pathway inhibitor and/or an RNA oligonucleotide encoding an IRF pathway inhibitor.

In some embodiments, one or more compositions of the present disclosure is configured deliver at least two innate immune repressor polypeptides as nucleic acid agents. For example, in some embodiments, one or more compositions of the present disclosure comprises an oligonucleotide (e.g., a RNA oligonucleotide) encoding a Vaccinia Virus Protein B14 polypeptide and an oligonucleotide (e.g., a RNA oligonucleotide) encoding Vaccinia Virus Protein K7 polypeptide. In some embodiments, one or more compositions of the present disclosure comprises an oligonucleotide (e.g., a RNA oligonucleotide) encoding a Vaccinia Virus Protein B14 polypeptide and an oligonucleotide (e.g., a RNA oligonucleotide) encoding a Vaccinia Virus Protein C6 polypeptide. In some embodiments, one or more compositions of the present disclosure comprises an oligonucleotide (e.g., a RNA oligonucleotide) encoding a Vaccinia Virus Protein K7 polypeptide and an oligonucleotide (e.g., a RNA oligonucleotide) encoding a Vaccinia Virus Protein C6 polypeptide. In some embodiments, one or more compositions of the present disclosure comprises an oligonucleotide (e.g., a RNA oligonucleotide) encoding a Vaccinia Virus Protein B14 polypeptide, an oligonucleotide (e.g., a RNA oligonucleotide) encoding a Vaccinia Virus Protein K7 polypeptide, and an oligonucleotide (e.g., a RNA oligonucleotide) encoding a Vaccinia Virus Protein C6 polypeptide.

RNA oligonucleotides (e.g., comprising a payload sequence and/or encoding an inhibitor of a NF-κB pathway and/or encoding an inhibitor of an IRF pathway) in any of nucleic acid expression systems and/or compositions described herein may be delivered as naked RNA oligonucleotides or complexed with a complexing agent, e.g., for protecting RNA oligonucleotides from degradation, and/or for facilitating cell delivery. Exemplary complexing agents include, but are not limited to lipids, polymers, or small arginine-rich peptide such as protamine. In some embodiments, RNA oligonucleotides (e.g., comprising a payload sequence and/or encoding an inhibitor of a NF-κB pathway and/or encoding an inhibitor of an IRF pathway) in any of nucleic acid expression systems and/or compositions described herein may be encapsulated, e.g., in liposomes or other suitable carriers.

In some embodiments, any of compositions described herein can be used in methods as described herein. For example, in some embodiments, a composition that delivers one or more inhibitors of a NF-κB pathway (e.g., an inhibitor of one or more components of IKK complex) can be for use in a method of enhancing expression and/or enhancing activity and/or reducing immunogenicity of a nucleic acid (e.g., an RNA oligonucleotide) comprising a payload sequence. In some embodiments, a composition that delivers one or more inhibitors of an IRF pathway (e.g., an inhibitor of a IKK-like complex, an inhibitor of a JAK-STAT pathway) can be used in a method of enhancing expression and/or enhancing activity and/or reducing immunogenicity of a nucleic acid (e.g., an RNA oligonucleotide) comprising a payload sequence. In some embodiments, a composition that delivers at least one inhibitor of a NF-κB pathway and at least one inhibitor of an IRF pathway is used in a method of enhancing expression and/or enhancing activity and/or reducing immunogenicity of a nucleic acid (e.g., an RNA oligonucleotide) comprising a payload sequence.

In some embodiments, any of compositions described herein can be a pharmaceutical composition.

Pharmaceutical Compositions

The present disclosure provides pharmaceutical compositions comprising any component, or combination of components, of a nucleic acid expression system as described herein. In some aspects, the present disclosure provides pharmaceutical compositions comprising an inhibitor of NF-κB pathway (e.g., as a polypeptide agent or a nucleic acid agent) and/or an inhibitor of an IRF pathway (e.g., as a polypeptide agent or a nucleic acid agent). In some embodiments, an RNA oligonucleotide encoding an inhibitor of NF-κB pathway and/or an inhibitor of an IRF pathway can be included in a pharmaceutical composition.

In some embodiments, a pharmaceutical composition comprises an inhibitor of NF-κB pathway and an inhibitor of an IRF pathway. In some embodiments, a pharmaceutical composition comprises an RNA oligonucleotide encoding an inhibitor of NF-κB pathway and an RNA oligonucleotide encoding an inhibitor of an IRF pathway.

In some embodiments, a pharmaceutical composition comprises at least one RNA oligonucleotide comprising a payload sequence and at least one inhibitor of a NF-κB pathway and/or IRF pathway.

In some embodiments, a pharmaceutical composition comprises any of the compositions described herein.

Pharmaceutical compositions as described herein can include a pharmaceutically acceptable carrier or excipient, which, can include any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference) discloses various excipients used in formulating pharmaceutical compositions and known techniques for the preparation thereof. Suitable pharmaceutically acceptable carriers include but are not limited to water, salt solutions (e.g., NaCl), saline, buffered saline, glycerol, sugars such as mannitol, sucrose, or others, dextrose, fatty acid esters, etc., as well as combinations thereof.

A pharmaceutical composition can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like), which do not deleteriously react with the active compounds or interfere with their activity. In certain embodiments, a water-soluble carrier suitable for intravenous administration is used. In some embodiments, a pharmaceutical composition can be sterile.

A suitable pharmaceutical composition, if desired, can also contain minor amounts of wetting or emulsifying agents, or pH buffering agents. A pharmaceutical composition can be a liquid solution, suspension, or emulsion.

A pharmaceutical composition can be formulated in accordance with the routine procedures as a pharmaceutical composition adapted for administration to human beings. The formulation of a pharmaceutical composition should suit the mode of administration. For example, in some embodiments, a composition for intravenous administration is typically a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where a pharmaceutical composition is to be administered by infusion, it can be dispensed with an infusion bottle containing sterile pharmaceutical grade water, saline or dextrose/water. Where a pharmaceutical composition is administered by injection, an ampule of sterile water for injection or saline can be provided so that the ingredients may be mixed prior to administration.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts or cells in vitro or ex vivo. Modification of pharmaceutical compositions suitable for administration to humans in order to render the compositions suitable for administration to various animals or cells in vitro or ex vivo is well understood, and the ordinarily skilled practitioner, e.g., a veterinary pharmacologist, can design and/or perform such modification with merely ordinary, if any, experimentation.

Formulations of the pharmaceutical compositions described herein may be prepared by any method known or hereafter developed in the art of pharmacology. In general, such preparatory methods include the step of bringing the active ingredient into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of a pharmaceutical composition described herein.

For example, a unit dose of a pharmaceutical composition comprises a predetermined amount of at least one RNA oligonucleotide comprising a payload sequence and/or at least one polypeptide or oligonucleotide inhibitor of a NF-κB pathway and/or at least one polypeptide or oligonucleotide inhibitor of an IRF pathway. In some embodiments, a unit dose of a pharmaceutical composition comprises at least one polypeptide or oligonucleotide inhibitor of a NF-κB pathway and/or at least one polypeptide or oligonucleotide inhibitor of an IRF pathway. In some embodiments, a unit dose of a pharmaceutical composition comprises at least one polypeptide or oligonucleotide inhibitor of a NF-κB pathway and at least one polypeptide or oligonucleotide inhibitor of an IRF pathway.

Relative amounts of any components in pharmaceutical compositions described herein, e.g., an RNA oligonucleotide comprising a payload sequence, at least one polypeptide or oligonucleotide inhibitor of a NF-κB pathway and/or at least one polypeptide or oligonucleotide IRF pathway (e.g., ones as described herein), a pharmaceutically acceptable excipient, and/or any additional ingredients can vary, depending upon the subject to be treated, target cells, and may also further depend upon the route by which the composition is to be administered.

Kits

Another aspect of the present disclosure further provides a pharmaceutical pack or kit comprising one or more containers filled with any component, or combination of components, of a nucleic acid expression system as described herein.

In some embodiments, a kit comprises one or more containers filled with a composition as described herein. For example, in some embodiments, a kit comprises a container including a composition that delivers an inhibitor of a NF-κB pathway (e.g., ones as described herein); and a container including a composition that delivers an inhibitor of an IRF pathway (e.g., ones as described herein).

In some aspects, a kit comprises a composition that delivers at least one oligonucleotide (e.g., RNA oligonucleotide) comprising a payload sequence and/or a composition that delivers at least one inhibitor of a NF-κB pathway and/or at least one inhibitor of an IRF pathway. In some embodiments, a kit comprises a composition that delivers at least one oligonucleotide (e.g., RNA oligonucleotide) comprising a payload sequence and/or a composition that delivers at least one oligonucleotide (e.g., RNA oligonucleotide) comprising a sequence that encodes an inhibitor of a NF-κB pathway and/or an inhibitor of an IRF pathway. In some embodiments, a kit comprises a composition that delivers at least one oligonucleotide (e.g., RNA oligonucleotide) comprising a payload sequence and/or a composition that delivers at least one polypeptide inhibitor of a NF-κB pathway and/or at least one polypeptide inhibitor of an IRF pathway.

In some aspects, a kit comprises a composition that delivers an inhibitor of a NF-κB pathway and/or an inhibitor of an IRF pathway. In some embodiments, a kit comprises a composition that delivers at least one oligonucleotide (e.g., RNA oligonucleotide) comprising a sequence that encodes an inhibitor of a NF-κB pathway and/or an inhibitor of an IRF pathway. In some embodiments, a kit comprises a composition that delivers a polypeptide inhibitor of a NF-κB pathway and/or at least one polypeptide inhibitor of an IRF pathway.

In some embodiments, a kit comprises a composition that delivers an inhibitor of a NF-κB pathway which inhibits IKK complex (e.g., an inhibitor of IKKα, IKKβ, and/or IKKγ). In some embodiments, a kit comprises a composition that delivers an IRF pathway inhibitor selected from: (i) an inhibitor of a IKK-like complex (e.g., an inhibitor of TBK1, IKKε, and/or DDX3), and (ii) an inhibitor of a JAK-STAT pathway (e.g., an inhibitor of STAT1, STAT2, and/or IRF9). In some embodiments, a kit comprises one or more compositions that deliver both an inhibitor of a NF-κB pathway (e.g., an inhibitor of IKK complex, e.g., an inhibitor of IKKα, IKKβ, and/or IKKγ) and an IRF pathway inhibitor selected from: (i) an inhibitor of a IKK-like complex (e.g., an inhibitor of TBK1, IKKε, and/or DDX3), and (ii) an inhibitor of a JAK-STAT pathway (e.g., an inhibitor of STAT1, STAT2, and/or IRF9).

In some embodiments, a kit comprises a composition including at least one oligonucleotide comprising a sequence that encodes an inhibitor of a NF-κB pathway and at least one oligonucleotide comprising a sequence that encodes an inhibitor of an IRF pathway. In some embodiments, a kit comprises a composition including an oligonucleotide encoding both a NF-κB pathway inhibitor and an IRF pathway inhibitor.

In some embodiments, a kit comprises a composition including at least one RNA oligonucleotide comprising a sequence that encodes an inhibitor of a NF-κB pathway and at least one RNA oligonucleotide comprising a sequence that encodes an inhibitor of a IRF pathway

Kits may be used in any applicable method, including, for example, cell treatment, therapeutically or diagnostically. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Cells

Cells comprising any embodiment of a nucleic acid expression system described herein are also provided herein. For example, in some embodiments, a cell comprises an oligonucleotide comprising a payload sequence (e.g., an RNA oligonucleotide) and an oligonucleotide comprising a sequence that encodes a NF-κB pathway inhibitor (e.g., ones described herein) and/or an IRF pathway inhibitor (e.g., ones described herein). In some embodiments, a payload sequence is introduced into cells via an RNA oligonucleotide comprising a payload sequence. In some embodiments, a NF-κB pathway inhibitor and/or an IRF pathway inhibitor introduced into cells via an RNA oligonucleotide. In some embodiments, a cell comprises an RNA oligonucleotide encoding a NF-κB pathway inhibitor and/or an IRF pathway inhibitor. In some embodiments, an RNA oligonucleotide encoding a NF-κB pathway inhibitor and/or an IRF pathway inhibitor is derived from a vaccinia virus.

Any cells can be chosen to express a payload sequence delivered via an oligonucleotide (e.g., an RNA oligonucleotide). In some embodiments, cells to be contacted with any of compositions or nucleic acid expression systems described herein can be wild-type cells, normal cells, diseased cells (e.g., cancer cells), or transgenic cells. In some embodiments, cells to be contacted with any of compositions or nucleic acid expression systems described herein can be eukaryotic cells (e.g., mammalian cells).

In some embodiments, cells as provided herein are cells that have been previously treated at least once or more (e.g., 1, 2, 3, 4, 5, 6, 7, 8, 9, 10 times or more) with one or more oligonucleotides. In some embodiments, oligonucleotides that are previously introduced into cells are DNA oligonucleotides. In some embodiments, oligonucleotides that are previously introduced into cells are RNA oligonucleotides (e.g., mRNA oligonucleotides).

### III. Methods of Use

The present disclosure provides, among other things, methods for using nucleic acid expression systems or compositions and/or components thereof as described herein. The present disclosure recognizes that challenges associated with cell treatment based on oligonucleotides involve high degradation of DNA oligonucleotides in cytoplasm and/or high immunogenicity associated with foreign RNA oligonucleotides to be introduced into cells.

The present disclosure also recognizes, among other things, that while using non-standard base chemistries may reduce immunogenicity of mRNA therapeutics, such modification may adversely affect efficiencies of translating mRNA to corresponding peptides or polypeptides in cells. Further, concerns with residual immune response that precludes repeated dosing and/or high-level dosing still remain. Therefore, there remains a need in the field for methods of delivering to target cells RNA oligonucleotides that minimize activation of myriad innate immune sensors while are still efficiently recognized by translational machinery.

The present disclosure, at least in part, addresses this need and provides methods by which innate immunity-triggered suppression of protein translation, mRNA degradation, and non-specific toxicity induced by oligonucleotides (e.g., RNA oligonucleotides) are reduced, thereby enhancing expression of oligonucleotides (e.g., RNA oligonucleotides) in cells. Further, higher doses and/or repeated doses of oligonucleotides (e.g., RNA oligonucleotides) can be applied to cells using any of methods described herein to improve or sustain expression of oligonucleotides (e.g., RNA oligonucleotides) without adversely inducing non-specific cell toxicity that would otherwise generally induced by any oligonucleotides (e.g., RNA oligonucleotides). These advantages can be beneficial for delivering and improving the effectiveness of oligonucleotide therapeutics (e.g., RNA therapeutics) and vaccines.

In some embodiments, a method comprises contacting a target cell with at least one of (i) an oligonucleotide (e.g., an RNA oligonucleotide) comprising a payload sequence (e.g., ones described herein); and (ii) a composition that delivers an NF-κB pathway inhibitor (e.g., ones described herein) and/or an IRF pathway inhibitor (e.g., ones described herein), such that the target cell receives both (i) and (ii). In some embodiments, a method comprises contacting a target cell with at least one of (i) an oligonucleotide (e.g., an RNA oligonucleotide) comprising a payload sequence (e.g., ones described herein); and (ii) a composition that delivers an NF-κB pathway inhibitor (e.g., ones described herein) and/or an IRF pathway inhibitor (e.g., ones described herein), so that the target cell is receiving a nucleic acid expression system of any embodiment in accordance with the present disclosure.

In some embodiments, a method comprises contacting a target cell with an oligonucleotide (e.g., an RNA oligonucleotide) comprising a payload sequence, where the target cell has previously been contacted with a composition (e.g., as described herein) that delivers a NF-κB pathway inhibitor and/or an IRF pathway inhibitor. In some embodiments, a method comprises contacting a target cell with composition that delivers an NF-κB pathway inhibitor and/or an IRF pathway inhibitor, where the target cell has previously been contacted with an oligonucleotide (e.g., an RNA oligonucleotide) comprising a payload sequence.

In some embodiments, a method comprises contacting a target cell with a composition that delivers at least one NF-κB pathway inhibitor (e.g., an IKK complex inhibitor, e.g., an inhibitor of IKKα, IKKβ, and/or IKKγ). In some embodiments, a method comprises contacting a target cell with one or more compositions that deliver at least one IRF pathway inhibitors, for example, in some embodiments selected from: (i) an inhibitor of an IKK-like complex (e.g., an inhibitor of TBK1, IKKε, and/or DDX3), and (ii) an inhibitor of a JAK-STAT pathway (e.g., an inhibitor of STAT1, STAT2, and/or IRF9). In some embodiments, a method comprises contacting a target cell with one or more compositions that deliver at least one inhibitor of a NF-κB pathway (e.g., an inhibitor of IKK complex, e.g., an inhibitor of IKKα, IKKβ, and/or IKKγ) and at least one IRF pathway inhibitor, for example in some embodiments selected from: (i) an inhibitor of an IKK-like complex (e.g., an inhibitor of TBK1, IKKε, and/or DDX3), and (ii) an inhibitor of a JAK-STAT pathway (e.g., an inhibitor of STAT1, STAT2, and/or IRF9).

In some embodiments, a method comprises contacting a target cell with a composition that delivers an oligonucleotide (e.g., an RNA oligonucleotide) comprising a payload sequence and with one or more compositions that deliver at least one NF-κB pathway inhibitor and/or at least one IRF pathway inhibitor. In some embodiments, a composition that delivers an oligonucleotide comprising a payload sequence and one or more compositions that deliver at least one NF-κB pathway inhibitor and/or at least one IRF pathway inhibitor are given to a target cell in a single composition. In some embodiments, a composition that delivers an oligonucleotide comprising a payload sequence and one or more compositions that deliver at least one NF-κB pathway inhibitor and/or at least one IRF pathway inhibitor are given to a target cell in separate compositions. In some embodiments, a method comprises contacting the target cell with 1, 2, 3, 4, 5, 6, 7, 8, 9, or 10 or more compositions, wherein each composition delivers one or more of: an oligonucleotide comprising a payload sequence; at least one NF-κB pathway inhibitor and/or at least one IRF pathway inhibitor.

In some embodiments, a method comprises contacting a target cell with a composition that delivers an oligonucleotide (e.g., an RNA oligonucleotide) comprising a payload sequence and with one or more compositions that deliver at least one NF-κB pathway inhibitor and at least one IRF pathway inhibitor. In some embodiments, at least one NF-κB pathway inhibitor and at least one IRF pathway inhibitor are delivered in the same composition. In some embodiments, at least one NF-κB pathway inhibitor and at least one IRF pathway inhibitor are delivered in separate compositions.

In some embodiments, provided herein is a method for enhancing expression and/or activity of a payload sequence delivered via an oligonucleotide (e.g., an RNA oligonucleotide), wherein the method comprises at least one of (i) contacting a target cell with an oligonucleotide (e.g., RNA oligonucleotide) comprising a payload sequence; and (ii) contacting the target cell with an oligonucleotide (e.g., RNA oligonucleotide) comprising a sequence that encodes at least one NF-κB pathway inhibitor and/or at least one IRF pathway inhibitor (e.g., ones described herein), such that the target cell receives both (i) and (ii).

In some embodiments, methods described herein are for enhancing expression and/or activity of a payload sequence in a target cell when the payload sequence is introduced into the target cell. In some embodiments, such a method includes contacting a target cell with one or more compositions that deliver at least one NF-κB pathway inhibitor (e.g., a IKK complex inhibitor, e.g., an inhibitor of IKKα, IKKβ, and/or IKKγ) and/or at least one IRF pathway inhibitor (e.g., an inhibitor of TBK1, IKKε, and/or DDX3; or e.g., an inhibitor of STAT1, STAT2, and/or IRF9). In some such embodiments, expression and/or activity of a payload sequence in a target cell is enhanced by at least 30% or more, including, e.g., at least 40%, at least 50%, at least 60%, at least 70%, at least 80%, at least 90%, at least 95%, or more, as compared to expression and/or activity of the same payload sequence in the target cell that has not been contacted with said one or more compositions that deliver at least one NF-κB pathway inhibitor and/or at least one IRF pathway inhibitor. In some embodiments, expression and/or activity of a payload sequence in a target cell is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to expression and/or activity of the same payload sequence in a target cell that has not been contacted with said one or more compositions that deliver at least one NF-κB pathway inhibitor and/or at least one IRF pathway inhibitor.

In some embodiments, provided herein is a method for reducing immunogenicity of an oligonucleotide (e.g., an RNA oligonucleotide) comprising a payload sequence upon delivery to a target cell, wherein the method comprises at least one of (i) contacting a target cell with an oligonucleotide (e.g., RNA oligonucleotide) comprising a payload sequence; and (ii) contacting the target cell with one or more compositions that deliver at least one NF-κB pathway inhibitor and/or at least one IRF pathway inhibitor (e.g., ones described herein), such that the target cell receives both (i) and (ii). In some embodiments, a target cell is contacted with one or more oligonucleotides (e.g., RNA oligonucleotides) each comprising a sequence that encodes at least one NF-κB pathway inhibitor and/or at least one IRF pathway inhibitor (e.g., ones described herein)

In some embodiments, methods described herein are for reducing immunogenicity an oligonucleotide (e.g., RNA oligonucleotide) comprising a payload sequence when the payload sequence is introduced into a target cell. In some embodiments, such methods include contacting a target cell with one or more compositions that deliver at least one NF-κB pathway inhibitor (e.g., a IKK complex inhibitor, e.g., an inhibitor of IKKα, IKKβ, and/or IKKγ) and/or at least one IRF pathway inhibitor (e.g., an inhibitor of TBK1, IKKε, and/or DDX3; or e.g., an inhibitor of STAT1, STAT2, and/or IRF9).

In some embodiments, immunogenicity of an oligonucleotide (e.g., RNA oligonucleotide) comprising a payload sequence is characterized by detecting level and/or activity of at least one or more pro-inflammatory cytokines. Pro-inflammatory cytokines include, but are not limited to TNF-α and IL-6. In some embodiments, immunogenicity of an oligonucleotide (e.g., RNA oligonucleotide) comprising the payload sequence is characterized by detecting degradation of an oligonucleotide (e.g., RNA oligonucleotide) comprising a payload sequence upon delivery to the cell. One of ordinary skill in the art will appreciate that other characterization methods for assessing immunogenicity of an oligonucleotide (e.g., RNA oligonucleotide) comprising a payload sequence may be used.

In some embodiments, a level or activity of a pro-inflammatory cytokine (e.g., TNF-α and/or IL-6) in a target cell is reduced by at least 30% or more, including, e.g., at least 40%, at least 50%, at least 60%, at least 70%, at least 80%, at least 90%, at least 95%, or more, as compared to a level of the same pro-inflammatory cytokine in a target cell contacted with the same payload sequence, which target cell has not been contacted with said one or more compositions that deliver at least one NF-κB pathway inhibitor and/or at least one IRF pathway inhibitor. In some embodiments, a level or activity of a pro-inflammatory cytokine in a target cell is reduced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to a level of the same pro-inflammatory cytokine in a target cell contacted with the same payload sequence, which target cell has not been contacted with said one or more compositions that deliver at least one NF-κB pathway inhibitor and/or at least one IRF pathway inhibitor.

In some embodiments, immunogenicity of an oligonucleotide (e.g., RNA oligonucleotide) comprising a payload sequence is characterized by detecting degradation of such an oligonucleotide (e.g., RNA oligonucleotide) comprising the payload sequence upon delivery to a cell. In some embodiments, a level of degradation of an oligonucleotide (e.g., RNA oligonucleotide) comprising a payload sequence in a target cell is reduced by at least 30% or more, including, e.g., at least 40%, at least 50%, at least 60%, at least 70%, at least 80%, at least 90%, at least 95%, or more, as compared to a level of degradation of an oligonucleotide (e.g., RNA oligonucleotide) comprising the same payload sequence, which target cell has not been contacted with said one or more compositions that deliver at least one NF-κB pathway inhibitor and/or at least one IRF pathway inhibitor. In some embodiments, a level of degradation of an oligonucleotide (e.g., RNA oligonucleotide) comprising a payload sequence in a target cell is reduced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to a level of degradation of an oligonucleotide (e.g., RNA oligonucleotide) comprising the same payload sequence, which target cell has not been contacted with said one or more compositions that deliver at least one NF-κB pathway inhibitor and/or at least one IRF pathway inhibitor.

In some embodiments, provided herein is a method for enhancing persistence or uptake of an oligonucleotide (e.g., RNA oligonucleotide) comprising a payload sequence upon delivery to a target cell, wherein the method comprises at least one of (i) contacting a target cell with an oligonucleotide (e.g., RNA oligonucleotide) comprising a payload sequence; and (ii) contacting the target cell with one or more compositions that deliver at least one NF-κB pathway inhibitor and/or at least one IRF pathway inhibitor (e.g., ones described herein), such that the target cell receives both (i) and (ii). In some embodiments, a target cell is contacted with one or more oligonucleotides (e.g., RNA oligonucleotides) each comprising a sequence that encodes at least one NF-κB pathway inhibitor and/or at least one IRF pathway inhibitor (e.g., ones described herein).

In some embodiments, methods described herein are for enhancing persistence or uptake of an oligonucleotide (e.g., RNA oligonucleotide) comprising a payload sequence in a target cell when a payload sequence is introduced into the target cell. In some embodiments, such methods include contacting a target cell with one or more compositions that deliver an NF-κB pathway inhibitor (e.g., a IKK complex inhibitor, e.g., an inhibitor of IKKα, IKKβ, and/or IKKγ) and/or an IRF pathway inhibitor (e.g., an inhibitor of TBK1, IKKε, and/or DDX3; or e.g., an inhibitor of STAT1, STAT2, and/or IRF9).

In some embodiments, persistence or uptake of an oligonucleotide comprising a payload sequence in a target cell is enhanced by at least 30% or more, including, e.g., at least 40%, at least 50%, at least 60%, at least 70%, at least 80%, at least 90%, at least 95%, or more, as compared to persistence or uptake of an oligonucleotide comprising the same payload sequence in a target cell, which target cell has not been contacted with said one or more compositions that deliver at least one NF-κB pathway inhibitor and/or at least one IRF pathway inhibitor. In some embodiments, persistence or uptake of an oligonucleotide comprising a payload sequence in a target cell is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to persistence or uptake of an oligonucleotide comprising the same payload sequence in a target cell, which target cell has not been contacted with said one or more compositions that deliver at least one NF-κB pathway inhibitor and/or at least one IRF pathway inhibitor. Accordingly, in some embodiments, provided herein is a method for enhancing persistence or uptake of an oligonucleotide comprising a payload sequence in a target cell comprising a payload sequence in a target cell, wherein the method comprises (a) contacting a target cell with an oligonucleotide (e.g., RNA oligonucleotide) comprising a payload sequence; and (b) contacting the target cell with an oligonucleotide (e.g., RNA oligonucleotide) comprising a sequence that encodes a NF-κB pathway inhibitor and/or an IRF pathway inhibitor (e.g., ones described herein).

In some embodiments, methods described herein are useful for enhancing viability of a target cell upon contacting with an oligonucleotide (e.g., an RNA oligonucleotide) comprising a payload sequence and one or more compositions that deliver at least one NF-κB pathway inhibitor and/or at least one IRF pathway inhibitor (e.g., ones as described herein). In some embodiments, such methods include contacting a target cell with one or more compositions (e.g., ones described herein) that deliver at least one NF-κB pathway inhibitor (e.g., an IKK complex inhibitor, e.g., an inhibitor of IKKα, IKKβ, and/or IKKγ) and/or at least one IRF pathway inhibitor (e.g., an inhibitor of TBK1, IKKε, and/or DDX3; or e.g., an inhibitor of STAT1, STAT2, and/or IRF9).

In some embodiments, viability of a target cell upon contacting with an oligonucleotide (e.g., RNA oligonucleotide) comprising a payload sequence and one or more compositions that delivers at least one NF-κB pathway inhibitor and/or at least one IRF pathway inhibitor (e.g., ones as described herein) is increased at least 30%, including, e.g., at least 40%, at least 50%, at least 60%, at least 70%, at least 80%, at least 90%, at least 95%, or more, as compared to viability of a target cell upon contacting with an oligonucleotide comprising the same payload sequence in the absence of one or more compositions that deliver the NF-κB pathway inhibitor and/or IRF pathway inhibitor. In some embodiments, viability of a target cell is enhanced by at least 1.1-fold or more, including, e.g., at least 1.5-fold, at least 2-fold, at least 2.5-fold, at least 3-fold, at least 3.5-fold, at least 4-fold, at least 5-fold, at least 10-fold, at least 20-fold, at least 30-fold, at least 40-fold, or more, as compared to viability of a target cell upon contacting with an oligonucleotide comprising the same payload sequence in the absence of one or more compositions that deliver the NF-κB pathway inhibitor and/or IRF pathway inhibitor.

In some embodiments, provided herein is a method for reducing non-specific toxicity induced in the target cell by an oligonucleotide comprising a payload sequence (e.g., an RNA oligonucleotide), wherein the method comprises at least one of (i) contacting a target cell with an oligonucleotide (e.g., RNA oligonucleotide) comprising a payload sequence; and (ii) contacting the target cell with one or more compositions that deliver at least one NF-κB pathway inhibitor and/or at least one IRF pathway inhibitor (e.g., ones described herein), such that the target cell receives both (i) and (ii). In some embodiments, a target cell is contacted with one or more oligonucleotides (e.g., RNA oligonucleotides) each comprising a sequence that encodes at least one NF-κB pathway inhibitor and/or at least one IRF pathway inhibitor (e.g., ones described herein).

In some embodiments, methods described herein are useful for reducing non-specific toxicity induced in the target cell by an oligonucleotide comprising a payload sequence (e.g., an RNA oligonucleotide). In some embodiments, such methods include contacting a target cell with one or more compositions (e.g., ones described herein) that deliver at least one NF-κB pathway inhibitor (e.g., an IKK complex inhibitor, e.g., an inhibitor of IKKα, IKKβ, and/or IKKγ) and/or at least one IRF pathway inhibitor (e.g., an inhibitor of TBK1, IKKε, and/or DDX3; or e.g., an inhibitor of STAT1, STAT2, and/or IRF9).

Methods described herein can be used for in vitro, ex vivo and in vivo applications. Thus, cells to which agents (e.g., oligonucleotide comprising a payload sequence and/or polypeptide and/or oligonucleotide inhibitor(s) of a NF-κB pathway and/or polypeptide and/or oligonucleotide inhibitor(s) of an IRF pathway) are delivered can be, for example, cells cultured in vitro or ex vivo, cells within a tissue, or cells present in a subject or organism. In some embodiments, said oligonucleotides are RNA oligonucleotides (e.g., mRNA oligonucleotides). In some embodiments, cells amenable to technologies provided herein can be cells that have been previously treated at least once or more (e.g., 1, 2, 3, 4, 5, 6, 7, 8, 9, 10 times or more) with one or more oligonucleotides. In some embodiments, oligonucleotides that are previously introduced into cells can be DNA oligonucleotides. In some embodiments, oligonucleotides that are previously introduced into cells can be RNA oligonucleotides (e.g., mRNA oligonucleotides).

Agents (e.g., oligonucleotide comprising a payload sequence and/or polypeptide and/or oligonucleotide inhibitor(s) of a NF-κB pathway and/or polypeptide and/or oligonucleotide inhibitor(s) of an IRF pathway) used in any methods described herein can be delivered to cells by any of known methods in the art, including, but not limited to, transfection into cells (e.g., via electroporation, chemical methods, etc.), delivery via particles (e.g., nanoparticles or liposomes), and/or administration to an organism (e.g., by any suitable administration route). In some embodiments, said oligonucleotides are RNA oligonucleotides (e.g., mRNA oligonucleotides).

In some embodiments, cells subjected to a method described herein are present in a subject. Therefore, in these embodiments, a target cell present in a subject is contacted with an oligonucleotide comprising a payload sequence by administering the oligonucleotide comprising the payload sequence to the subject. In some embodiments, a target cell present in a subject is contacted with a composition that delivers an inhibitor of a NF-κB pathway and/or an inhibitor of an IRF pathway.

In some embodiments, methods, nucleic acid expression systems, and/or compositions described herein can be used for delivering an oligonucleotide (e.g., RNA oligonucleotide) to a target cell for a gene therapy or RNA oligonucleotide therapy in a subject. In some embodiments, a subject is a mammalian subject. In some embodiments, a subject is a human subject.

In some embodiments, a target cell to be subjected to a method, nucleic acid expression system, and/or composition described herein is isolated from a subject. In some embodiments, a target cell can be autologous to a subject (i.e., from a subject). In some embodiments, a target cell can be non-autologous (i.e., allogeneic or xenogeneic) to a subject.

In some embodiments, a target cell (e.g., for in vitro, ex vivo, or in vivo applications described herein) is contacted with an oligonucleotide (e.g., RNA oligonucleotide) comprising a payload sequence and a composition that delivers an inhibitor of a NF-κB pathway and/or an inhibitor of an IRF pathway concurrently. In some embodiments, a target cell (e.g., for in vitro, ex vivo, or in vivo applications described herein) is contacted with an oligonucleotide (e.g., RNA oligonucleotide) comprising a payload sequence and composition that delivers an inhibitor of a NF-κB pathway and/or an inhibitor of an IRF pathway separately. For example, in some embodiments, an RNA oligonucleotide comprising a sequence that encodes a payload sequence is delivered to a target cell, and an RNA oligonucleotide encoding an inhibitor of a NF-κB pathway and/or an inhibitor of an IRF pathway is delivered to the target cell at a later time. In some embodiments, an RNA oligonucleotide comprising a payload sequence is delivered to a target cell during a time when innate immunity pathway is attenuated (e.g., temporarily attenuated by at least 10% or more including, e.g., at least 20%, at least 30%, at least 40%, or more) by a polypeptide or oligonucleotide inhibitor of a NF-κB pathway and/or a polypeptide or oligonucleotide inhibitor of an IRF pathway. For example, in some embodiments, an oligonucleotide (e.g., RNA oligonucleotide) comprising a payload sequence is delivered to a target cell 30 min, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 1 week, 2 weeks, 3 weeks, 4 weeks, 6 weeks, or 8 weeks after a polypeptide or oligonucleotide inhibitor of a NF-κB pathway and/or a polypeptide or oligonucleotide inhibitor of an IRF pathway is delivered.

In some embodiments, a composition comprising at least one RNA oligonucleotide sequence that encodes an inhibitor of a NF-κB pathway and/or an inhibitor of an IRF pathway is delivered to a target cell that has been contacted with an RNA oligonucleotide comprising a payload sequence, such that the target cell receives both.

In some embodiments, a composition comprising an RNA oligonucleotide comprising a payload sequence is administered to a target cell that has been contacted with at least one RNA oligonucleotide sequence that encodes an inhibitor of a NF-κB pathway and/or an inhibitor of an IRF pathway, such that the target cell receives both.

Other features of the invention will become apparent in the course of the following descriptions of exemplary embodiments, which are given for illustration of the invention and are not intended to be limiting thereof.

### EXEMPLIFICATION

### Example 1—Identification of Inhibitors of NF-κB pathway

The present Example describes identification of exemplary NF-κB pathway inhibitors. Specifically, a panel of candidate innate immune repressor proteins were screened for their ability to inhibit NF-κB pathway activity in cell culture using a NF-κB reporter construct. Cells expressing a NF-κB promoter reporter were transfected with an exemplary mRNA oligonucleotide encoding a candidate innate immune repressor protein. Reporter activation, toxicity and cell viability were each assessed.

mRNA Synthesis of Candidate Proteins

Transcription templates of candidate proteins (e.g., candidate immune repressor proteins) were synthesized as gBlock dsDNA fragments (Integrated DNA Technologies) and PCR amplified using a commercially available polymerase (e.g., Herculase II polymerase (Agilent)) and suitable primers. For example, amplification with the following primers and an annealing temperature of 50° C.:

T7-GGG fwd: gaattTAATACGACTCACTATAGGGcttgttctttttgcagaagc (SEQ ID NO: 1)

120pA_rev:
(SEQ ID NO: 2)
TTTTTTTTTTTTTTTTTTTTTTTTTTTTTTTTTTTTTTTTTTTTTTTTTT
TTTTTTTTTTTTTTTTTTTTTTTTTTTTTTTTTTTTTTTTTTTTTTTTTT
TTTTTTTTTTTTTTTTTTTTagaatgtgaagaaactttctttttattag

A panel of RNA oligonucleotides encoding exemplary vaccinia virus candidate innate immune repressor proteins were prepared. PCR products were cleaned using commercially available methods, such as a QIAquick Purification Kit (QIAGEN), prior to RNA transcription. RNA was synthesized from the DNA templates using an RNA polymerase (e.g., a T7 polymerase). In some embodiments, RNA purification can be carried out using any methods known in the art, for example: purification using the 500 μg capacity Monarch RNA Cleanup Kit (New England Biolabs), followed by treatment with DNAse I, and subsequent purification again using 500 μg Monarch columns.

RNA capping was performed using known methods including, e.g., co-transcriptional or post-transcriptional capping approaches. In some embodiments, co-transcriptional capping may be performed by inclusion of a known co-transcriptional capping agent, e.g., CleanCap® Reagent AG (TriLink BioTechnologies) in an in vitro transcription reaction. In some embodiments, post-transcriptional capping may be performed after RNA was synthetized. For example, in some embodiments, exemplary reaction conditions for post-transcriptional RNA capping can include the following: 20 μL reactions containing 6.5 μg RNA, 0.5 mM GTP, 0.2 mM S-adenosylmethionine, 10 units Vaccinia Capping Enzymes (New England Biolabs), 50 units Cap 2′-O-Methyltransferase (New England Biolabs), 5 mM KCl, 1 mM MgCl2, 2 mM DTT, and 50 mM Tris-HCl pH 8 for 1 hr at 37° C. Capped mRNAs can then be purified using commercially available methods, such as, for example, the 10 μg capacity Monarch RNA Cleanup Kit, treated with Quick CIP (New England Biolabs) for 10 min at 37° C., and purified again using 10 μg Monarch columns. mRNA concentrations were determined using methods known in the art, such as spectrophotometry, e.g., using a NanoDrop spectrophotometer (Thermo Fisher).

### NF-κB Reporter Assay Screen

Screening of exemplary RNA oligonucleotides encoding vaccinia virus candidate innate immune repressor proteins was performed using an in vitro cell culture assay using a NF-κB reporter construct. Any appropriate cells and culture conditions known in the art can be used for screening. For example, A549-Dual (InvivoGen) were cultured in high glucose GlutaMAX Dulbecco's Modified Eagle Medium (Thermo Fisher) supplemented with 10% heat-inactivated fetal bovine serum, 100 units/mL penicillin, 100 μg/mL streptomycin, 10 μg/mL blasticidin, and 100 μg/mL zeocin and maintained at 37° C. and 5% CO₂.

Cells were transfected with exemplary RNA oligonucleotides encoding vaccinia virus candidate innate immune repressor proteins using known methods. Exemplary reaction conditions include complexing 60 ng of each mRNA mixture with 90 nL MessengerMAX (Thermo Fisher) in 10 μL total volume of Opti-MEM (Thermo Fisher). Each mRNA mixture consisted of either 60 ng FLuc mRNA or 30 ng FLuc mRNA and 30 ng of the particular helper mRNA. Reverse transfections were carried out by mixing each mRNA/MessengerMAX complex with approximately 1,000 cells in a well of a 96-well plate. Transfections were performed in triplicate.

Immunogenicity was measured 37 hr after transfection. Viability and expression levels and reporter expression were assayed using commercially available methods. For example, viability and expression was assessed using a One-Glo+Tox Luciferase Reporter and Cell Viability Assay kit (Promega), NF-κB promoter activation was measured using a QUANTI-Blue assay (InvivoGen), and IRF reporter activation was measured using a QUANTI-Luc assay (Invivogen). Assay read-outs can be assessed using a commercially available spectrophotometer. For example, all measurements were taken on a Spectramax i3x plate reader.

Screening and Identification of Exemplary NF-κB pathway inhibitors

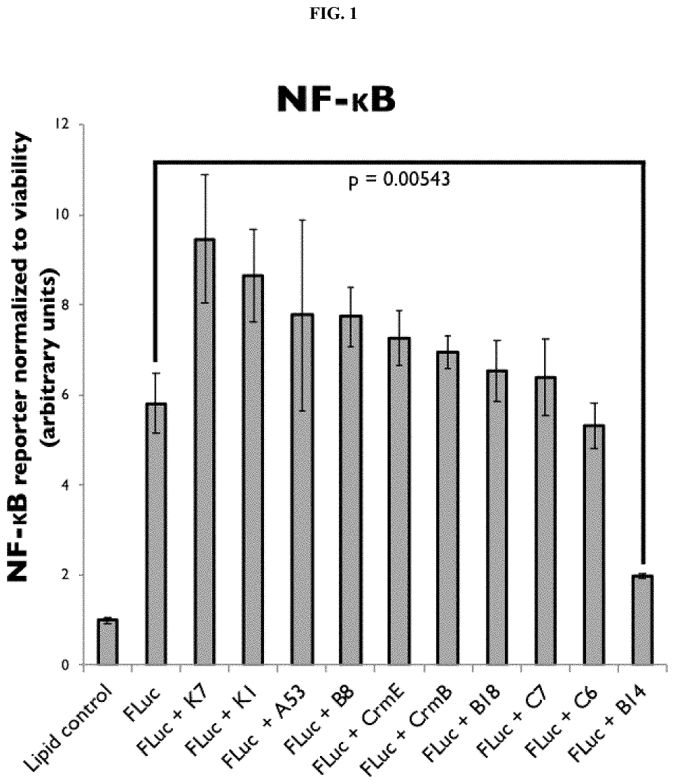

Using an exemplary screen as described above, B14-encoding mRNA was identified as a potent NF-κB inhibitor. Results are shown in FIG. 1. Mechanistic studies suggest that B14 protein directly binds and inhibits IKKβ. (Tang et al. (2018) “Mechanism of vaccinia viral protein B14 mediated inhibition of IκB kinase β activation,” J. Biol. Chem. 293: 10344-10352, the contents of which are incorporated by reference in their entirety for purposes described herein).

An exemplary B14 DNA template sequence (with UTRs) is shown as follows:

(SEQ ID NO: 3)
CTTGTTCTTTTTGCAGAAGCTCAGAATAAACGCTCAACTTTGGCCACCa
tgacggccaactttagtacccacgtcttttcaccacagcactgtggatg
tgacagactgaccagtattgatgacgtcagacaatgtttgactgaatat
atttattggtcgtcctatgcataccgcaacaggcaatgcgctggacaat
tgtattccacactcctctcttttagagatgatgcggaattagtgttcat
cgacattcgcgagctggtaaaaaatatgccgtgggatgatgtcaaagat
tgtgcagaaatcatccgttgttatataccggatgagcaaaaaaccatca
gagagatttcggccatcatcggactttgtgcatatgctgctacttactg
gggaggtgaagaccatcccactagtaacagtctgaacgcattgtttgtg
atgcttgagatgctcaattacgtggattataacatcatattccggcgta
tgaatTAATGATAGACCAGCCTCAAGAACACCCGAATGGAGTCTCTAAG
CTACATAATACCAACTTACACTTTACAAAATGTTGTCCCCCAAAATGTA
GCCATTCGTATCTGCTCCTAATAAAAAGAAAGTTTCTTCACATTCT

The capitalized sequences are the Xenopus laevis beta-globin 5′ and 3′ UTR sequences. The sequence shown in SEQ ID NO: 4 below was taken from the complete genome of vaccinia virus Western Reserve strain (GenBank accession AY243312.1).

An exemplary B14 DNA template sequence (lacking UTRs) is shown as follows:

(SEQ ID NO: 4)
atgacggccaactttagtacccacgtcttttcaccacagcactgtggat
gtgacagactgaccagtattgatgacgtcagacaatgtttgactgaata
tatttattggtcgtcctatgcataccgcaacaggcaatgcgctggacaa
ttgtattccacactcctctcttttagagatgatgcggaattagtgttca
tcgacattcgcgagctggtaaaaaatatgccgtgggatgatgtcaaaga
ttgtgcagaaatcatccgttgttatataccggatgagcaaaaaaccatc
agagagatttcggccatcatcggactttgtgcatatgctgctacttact
ggggaggtgaagaccatcccactagtaacagtctgaacgcattgtttgt
gatgcttgagatgctcaattacgtggattataacatcatattccggcgt
atgaat

An exemplary B14 amino acid sequence (e.g., from vaccinia virus) is shown as follows:

(SEQ ID NO: 5)
MTANFSTHVFSPQHCGCDRLTSIDDVRQCLTEYIYWSSYAYRNRQCAGQ
LYSTLLSFRDDAELVFIDIRELVKNMPWDDVKDCAEIIRCYIPDEQKTI
REISAIIGLCAYAATYWGGEDHPTSNSLNALFVMLEMLNYVDYNIIFRR
MN

The present disclosure encompasses a recognition that other inhibitors of IKKβ can substitute for B14 in inhibition of NF-κB signaling.

### Example 2—Identification of Inhibitors of IRF Pathway

The present Example describes identification of exemplary IRF pathway inhibitors. Specifically, a panel of candidate innate immune repressor proteins were screened for their ability to inhibit IRF pathway activity in cell culture using an IRF reporter construct. While B14 significantly reduced NF-κB activation caused by mRNA transfection as described in Example 1 above, significant activation of an IRF reporter was still observed. The present disclosure provides the insight that a second inhibitor that inhibits the IRF axis may enhance suppression of innate immune response and thus further reduce immunogenicity of nucleic acids (e.g., RNA oligonucleotides) introduced into cells. Accordingly, the present example describes a screen of pairwise combinations of candidate inhibitors of NF-κB and IRF.

Cells expressing an IRF promoter reporter and a NF-κB promoter reporter were transfected with an exemplary inhibitor of NF-κB signaling (e.g., a B14 mRNA oligonucleotide, e.g., as described in Example 1) and a second candidate innate immune repressor (e.g., vaccinia virus innate immune modulators). Pairwise combinations of candidate innate immune repressors were analyzed by reporter activation, toxicity and cell viability.

mRNA Synthesis of Candidate Proteins

Transcription templates of candidate proteins (e.g., candidate immune repressor proteins) were synthesized as gBlock dsDNA fragments (Integrated DNA Technologies) and PCR amplified using a commercially available polymerase (e.g., Herculase II polymerase (Agilent)) and suitable primers. For example, amplification with exemplary primers as follows and an annealing temperature of 50° C.

T7-AGG_fwd:
(SEQ ID NO: 6)
gaattTAATACGACTCACTATAAGGcttgttctttttgcagaagc

120 pA_rev primer as described above in Example 1 (SEQ ID NO: 2).

The PCR products were cleaned up using QIAquick Purification Kit prior to RNA transcription. RNA was synthesized from the DNA templates using an RNA polymerase (e.g., a T7 polymerase). Co-transcriptional capping was performed by inclusion of a co-transcriptional capping agent, e.g., CleanCap® Reagent AG (TriLink BioTechnologies) in an in vitro transcription reaction. RNA purification can be carried out using any methods known in the art. For example, the transcribed RNAs were purified using the 500 μg capacity Monarch RNA Cleanup Kit, treated with DNAse I, and purified again using 50 μg-capacity Monarch columns. mRNAs were then treated with Quick CIP for 10 min at 37° C. and purified using 50 μg Monarch columns. Concentrations were determined using a NanoDrop spectrophotometer.

A panel of RNA oligonucleotides encoding exemplary vaccinia virus candidate innate immune repressor proteins were prepared as discussed above.

mRNAs were then treated with Quick CIP for 10 min at 37° C. and purified using commercially available (50 μg Monarch) columns. Concentrations were determined by spectrophotometry (e.g., using a NanoDrop spectrophotometer (Thermo Fisher)).

### IRF and NF-κB Reporter Assay Screen

Pairwise screening of exemplary RNA oligonucleotides encoding vaccinia virus candidate innate immune repressor proteins in combination with an exemplary inhibitor of NF-κB signaling (e.g., a B14 mRNA oligonucleotide, e.g., as described in Example 1) was performed using an in vitro cell culture assay. Any appropriate cells and culture conditions known in the art can be used for screening.

For example, A549-Dual (InvivoGen) were cultured in high glucose GlutaMAX Dulbecco's Modified Eagle Medium (Thermo Fisher) supplemented with 10% heat-inactivated fetal bovine serum, 100 units/mL penicillin, 100 μg/mL streptomycin, 10 μg/mL blasticidin, and 100 μg/mL zeocin and maintained at 37° C. and 5% CO₂.

Cells were plated to a 96-well at 6,000 cells/well 1 d prior to transfection. Exemplary transfection conditions are as follows: 200 ng of each mRNA (either 200 ng FLuc mRNA or 100 ng FLuc and 100 ng of equimolar amounts of each helper mRNA combination) were transfected using TransIT-mRNA Transfection Kit (MirusBio) using 2 μL mRNA Boost Reagent: 1 μg mRNA and 2 μL TrainsIT-mRNA Reagent: 1 μg mRNA. Transfections were performed in duplicate.

Immunogenicity was measured 20 hr after transfection. Viability and expression levels and reporter expression were assayed using commercially available methods. For example, viability and expression levels were assayed using the One-Glo+Tox Luciferase Reporter and Cell Viability Assay kit, NF-κB promoter activation was measured using the QUANTI-Blue assay, and ISRE reporter activation was measured using the QUANTI-Luc assay. Assay read-outs can be assessed using a commercially available spectrophotometer. For example, all measurements were taken on a Spectramax i3x plate reader.

Screening and Identification of Exemplary IRF pathway inhibitors

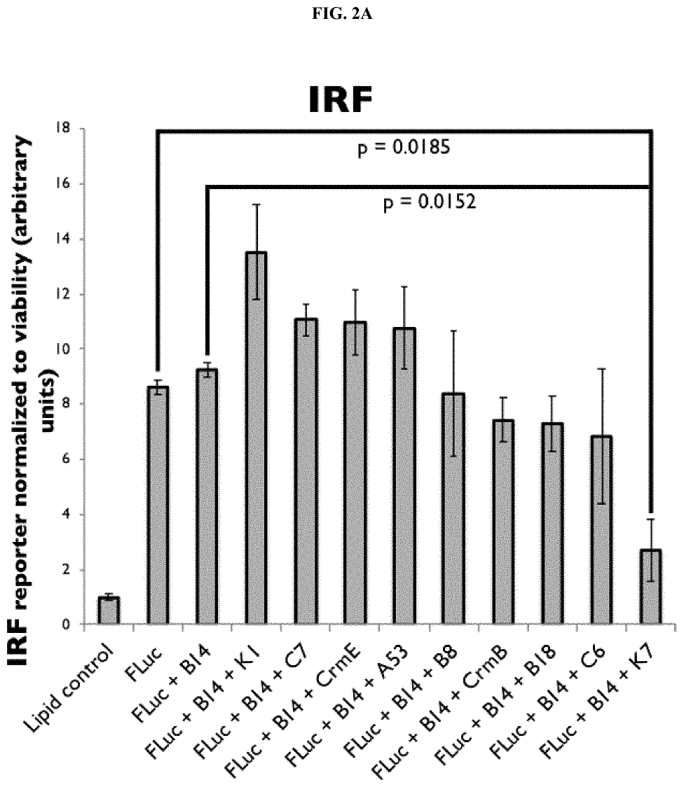

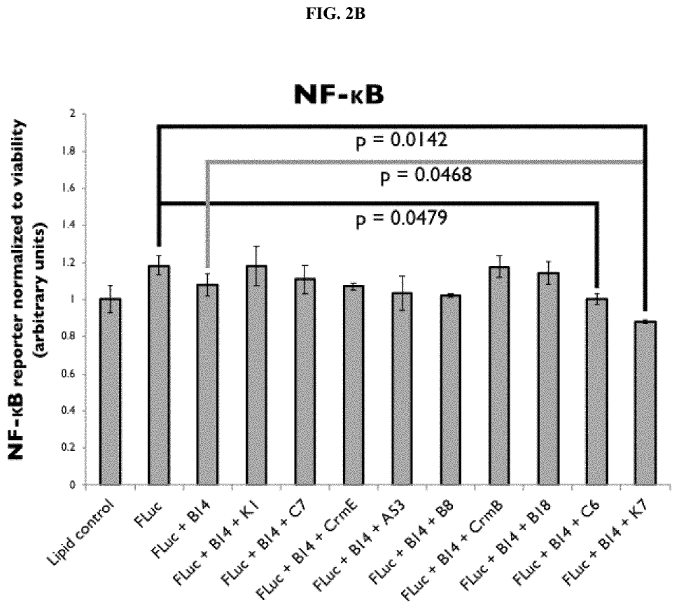

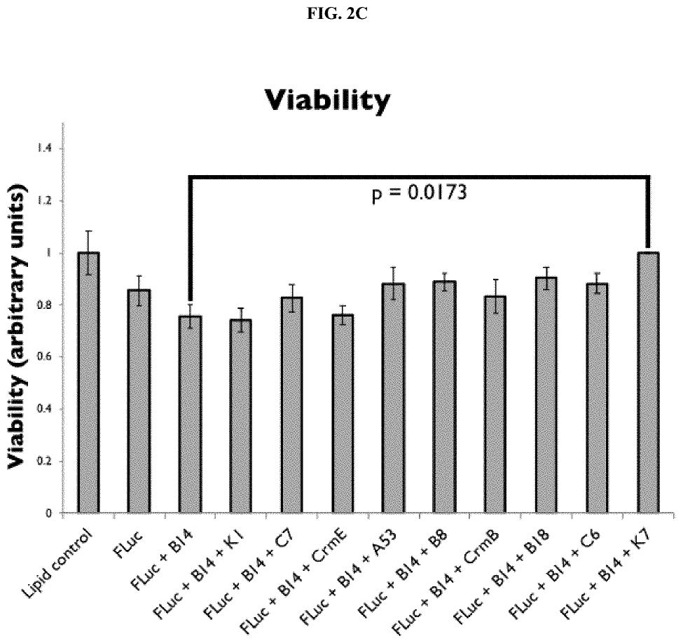

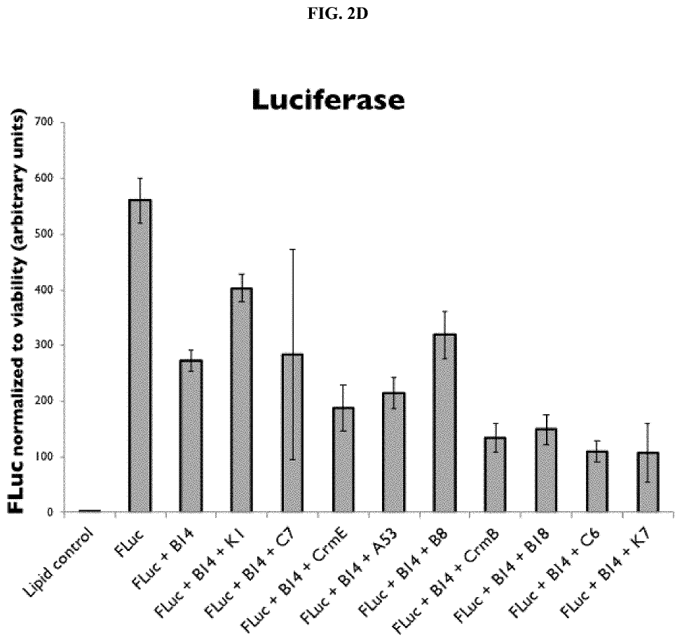

Using an exemplary screen as described above, it was demonstrated that a pairwise combination of B14 and K7 can be used to simultaneously suppress both NF-κB and IRF activation. Results are shown in FIGS. 2A-2D.

An exemplary K7 DNA template sequence (with UTRs) is shown as follows:

(SEQ ID NO: 7)
CTTGTTCTTTTTGCAGAAGCTCAGAATAAACGCTCAACTTTGGCCACCa
tggcgactaaattagattatgaggatgctgttttttactttgtggatga
tgataaaatatgtagtcgcgactccatcatcgatctaatagatgaatat
attacgtggagaaatcatgttatagtgtttaacaaagatattaccagtt
gtggaagactgtacaaggaattgatgaagttcgatgatgtcgctatacg
gtactatggtattgataaaattaatgagattgtcgaagctatgagcgaa
ggagaccactacatcaattttacaaaagtccatgatcaggaaagtttat
tcgctaccataggaatatgtgctaaaatcactgaacattggggatacaa
aaagatttcagaatctagattccaatcattgggaaacattacagatctg
atgaccgacgataatataaacatcttgatactttttctagaaaaaaaat
tgaatTAATGATAGACCAGCCTCAAGAACACCCGAATGGAGTCTCTAAG
CTACATAATACCAACTTACACTTTACAAAATGTTGTCCCCCAAAATGTA
GCCATTCGTATCTGCTCCTAATAAAAAGAAAGTTTCTTCACATTCT

An exemplary K7 DNA template sequence (lacking UTRs) is shown as follows:

(SEQ ID NO: 8)
atggcgactaaattagattatgaggatgctgttttttactttgtggatg
atgataaaatatgtagtcgcgactccatcatcgatctaatagatgaata
tattacgtggagaaatcatgttatagtgtttaacaaagatattaccagt
tgtggaagactgtacaaggaattgatgaagttcgatgatgtcgctatac
ggtactatggtattgataaaattaatgagattgtcgaagctatgagcga
aggagaccactacatcaattttacaaaagtccatgatcaggaaagttta
ttcgctaccataggaatatgtgctaaaatcactgaacattggggataca
aaaagatttcagaatctagattccaatcattgggaaacattacagatct
gatgaccgacgataatataaacatcttgatactttttctagaaaaaaaa
ttgaat

The sequence shown in SEQ ID NO: 8 above was taken from a partial vaccinia virus genomic sequence (GenBank accession D00382.1), and encodes the following amino acid sequence:

(SEQ ID NO: 9)
MATKLDYEDAVFYFVDDDKICSRDSIIDLIDEYITWRNHVIVFNKDITS
CGRLYKELMKFDDVAIRYYGIDKINEIVEAMSEGDHYINFTKVHDQESL
FATIGICAKITEHWGYKKISESRFQSLGNITDLMTDDNINILILFLEKK
LN

Without wishing to be bound by theory, K7 is believed to inhibit DEAD box protein 3 (DDX3) and thereby prevent TBK1/IKKε activation of IRF-3 and IRF-7 (Schroder et al., EMBO J 2008, the contents of which are incorporated by reference in their entirety for purposes described herein), as depicted in FIG. 3. The present disclosure encompasses a recognition that other inhibitors of DDX3, TBK1, or IKKε can substitute for K7 for use in inhibiting IRF activation by synthetic RNA.

Also shown in FIG. 2B, significant NF-κB repression was observed when B14 was jointly expressed with C6. Without wishing to be bound by theory, C6 is thought to bind to STAT2 (Stuart et al., PLoS Pathogens 2016, the contents of which are incorporated by reference in their entirety for purposes described herein), which may allow it to inhibit interferon-dependent activation of JAK/STAT signaling, as depicted in FIG. 4.

An exemplary C6 DNA template sequence (with UTRs) is shown as follows:

(SEQ ID NO: 10)
CTTGTTCTTTTTGCAGAAGCTCAGAATAAACGCTCAACTTTGGCCACCa
tgaatgcgtataataaagccgattcgttttctttagagtctgattctat
caaagatgttatacacgattatatttgttggctcagtatgactgatgaa
atgagaccatctatcggaaacgtctttaaagcgatggaaacgtttaaga
tagacgcggttagatattacgatggtaacatatatgaattagctaaaga
tataaatgcgatgtcgtttgacggttttataagatctctacaaactatc
gcttcaaagaaagataaactcactgtttatggaaccatgggactgctgt
ctattgtcgtagatattaacaaaggttgtgatatatccaatatcaagtt
cgctgccggaataatcattttaatggagtatatttttgatgacacggat
atgtctcatcttaaagtagcactctatcgtagaatacagagacgtgatg
atgtagatagaTAATGATAGACCAGCCTCAAGAACACCCGAATGGAGTC
TCTAAGCTACATAATACCAACTTACACTTTACAAAATGTTGTCCCCCAA
AATGTAGCCATTCGTATCTGCTCCTAATAAAAAGAAAGTTTCTTCACAT
TCT

An exemplary C6 DNA template sequence (lacking UTRs) is shown as follows:

(SEQ ID NO: 11)
atgaatgcgtataataaagccgattcgttttctttagagtctgattcta
tcaaagatgttatacacgattatatttgttggctcagtatgactgatga
aatgagaccatctatcggaaacgtctttaaagcgatggaaacgtttaag
atagacgcggttagatattacgatggtaacatatatgaattagctaaag
atataaatgcgatgtcgtttgacggttttataagatctctacaaactat
cgcttcaaagaaagataaactcactgtttatggaaccatgggactgctg
tctattgtcgtagatattaacaaaggttgtgatatatccaatatcaagt
tcgctgccggaataatcattttaatggagtatatttttgatgacacgga
tatgtctcatcttaaagtagcactctatcgtagaatacagagacgtgat
gatgtagataga

The sequence shown in SEQ ID NO: 11 above was taken from C6L sequence from vaccinia virus Western Reserve Strain (GenBank accession EF051270.1), and encodes the following amino acid sequence:

(SEQ ID NO: 12)
MNAYNKADSFSLESDSIKDVIHDYICWLSMIDEMRPSIGNVFKAMETFK
IDAVRYYDGNIYELAKDINAMSFDGFIRSLQTIASKKDKLIVYGTMGLL
SIVVDINKGCDISNIKFAAGIIILMEYIFDDTDMSHLKVALYRRIQRRD
DVDR

### Example 3—Expression of a Payload RNA Oligonucleotide when Co-Delivered with an Inhibitor of NF-κB Pathway and/or an Inhibitor of IFR Pathway

The present Example describes co-delivery of an RNA oligonucleotide comprising a model payload sequence to target cells with one or more compositions that deliver an inhibitor of NF-κB pathway signaling and/or an inhibitor of IFR pathway signaling.

In particular, this experiment assesses expression of a model reporter payload oligonucleotide in a target cell when co-transfected or sequentially transfected with oligonucleotide constructs encoding an inhibitor of NF-κB pathway signaling (e.g., a viral innate immune repressor such as e.g., B14) and/or an inhibitor of IFR pathway signaling (e.g., a viral innate immune repressor such as e.g., K7 and/or C6), a combination thereof, or a control oligonucleotide construct.

In some embodiments, co-delivery of a payload oligonucleotide with one or more RNA oligonucleotides (e.g., mRNA oligonucleotides) comprising a sequence that encodes an exemplary inhibitor of NF-κB pathway signaling (e.g., a viral innate immune repressor such as e.g., B14) alone or in combination with one or more inhibitors of IFR pathway signaling (e.g., a viral innate immune repressor such as e.g., K7 and/or C6) may improve expression of RNA oligonucleotide comprising a model payload sequence in cells in vitro. In some embodiments, co-delivery of a payload oligonucleotide with one or more RNA oligonucleotides (e.g., mRNA oligonucleotides) comprising a sequence that encodes an exemplary inhibitor of NF-κB pathway signaling (e.g., a viral innate immune repressor such as e.g., B14) alone or in combination with one or more inhibitors of IFR pathway signaling (e.g., a viral innate immune repressor such as e.g., K7 and/or C6) may improve cell viability in vitro after transfection, which in some embodiments, may allow repeated dosing of a payload oligonucleotide.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A nucleic acid expression system comprising:
(i) an RNA oligonucleotide comprising a payload sequence, and
(ii) at least one composition that delivers an inhibitor of a NF-κB pathway and/or an inhibitor of an IRF pathway.

2. The nucleic acid expression system of claim 1, wherein the RNA oligonucleotide (i) is present in a composition that is separate from the at least one composition that delivers an inhibitor of a NF-κB pathway and/or an inhibitor of an IRF pathway.

3. The nucleic acid expression system of claim 1, wherein the at least one composition that delivers an inhibitor of a NF-κB pathway and/or an inhibitor of an IRF pathway includes one of the following compositions:
a. a polypeptide inhibitor of a NF-κB pathway or a nucleic acid (e.g., an RNA oligonucleotide) comprising a sequence that encodes an inhibitor of a NF-κB pathway;
b. a polypeptide inhibitor of an IRF pathway or a nucleic acid (e.g., an RNA oligonucleotide) comprising a sequence that encodes an inhibitor of an IRF pathway;
c. a polypeptide inhibitor of a NF-κB pathway and a polypeptide inhibitor of an IRF pathway;
d. a polypeptide inhibitor of a NF-κB pathway and a nucleic acid (e.g., an RNA oligonucleotide) comprising a sequence that encodes an inhibitor of an IRF pathway;
e. a nucleic acid (e.g., an RNA oligonucleotide) comprising a sequence that encodes an inhibitor of a NF-κB pathway and a polypeptide inhibitor of an IRF pathway; and
f. a nucleic acid (e.g., an RNA oligonucleotide) comprising a sequence that encodes an inhibitor of a NF-κB pathway and a nucleic acid (e.g., an RNA oligonucleotide) comprising a sequence that encodes an inhibitor of an IRF pathway.

4. The nucleic acid expression system of claim 3, wherein the at least one composition that delivers an inhibitor of a NF-κB pathway and/or an inhibitor of an IRF pathway includes an RNA oligonucleotide comprising a sequence that encodes an inhibitor of a NF-κB pathway and an RNA oligonucleotide comprising a sequence that encodes an inhibitor of an IRF pathway.

5. The nucleic acid expression system of claim 1, wherein the RNA oligonucleotide comprising the sequence that encodes an inhibitor of a NF-κB pathway and the RNA oligonucleotide comprising a sequence that encodes an inhibitor of an IRF pathway are present in the same composition.

6. (canceled)

7. The nucleic acid expression system of claim 1, wherein the RNA oligonucleotide of (i) or (ii) is a synthetic RNA oligonucleotide.

8. (canceled)

9. The nucleic acid expression system of claim 1, wherein the RNA oligonucleotide of (i) or (ii) is a messenger RNA (mRNA) oligonucleotide.

10.-11. (canceled)

12. The nucleic acid expression system of claim 1, wherein the inhibitor of a NF-κB pathway is or comprises:
(a) a viral polypeptide, or a nucleic acid encoding the viral polypeptide; and/or
(b) a polypeptide agent that inhibits activity and/or formation of IκB kinase (IKK) complex, or a nucleic acid encoding the polypeptide agent.

13. (canceled)

14. The nucleic acid expression system of claim 12, wherein the polypeptide agent that inhibits activity and/or formation of IKK complex is or comprises a polypeptide agent that binds to and/or inhibits activity and/or interaction of at least one of an IKKα subunit, an IKKβ subunit, and an IKKγ subunit.

15-16. (canceled)

17. The nucleic acid expression system of claim 1, wherein the inhibitor of an IRF pathway is or comprises:
(a) a viral polypeptide, or a nucleic acid encoding the viral polypeptide; and/or
(b) a polypeptide agent that inhibits activity and/or formation of a complex comprising TANK-binding kinase 1 (TBK1) and IκB kinase ε (IKKε), or a nucleic acid encoding the polypeptide agent.

18. (canceled)

19. The nucleic acid expression system of claim 17, wherein the polypeptide agent that inhibits activity and/or formation of the TBK1/IKKε complex is or comprises a polypeptide agent that binds to and/or inhibits activity and/or interaction of DEAD box protein 3 (DDX3) with the TBK1/IKKε complex.

20.-21. (canceled)

22. The nucleic acid expression system of claim 1, wherein the inhibitor of an IRF pathway is or comprises an inhibitor of a JAK-STAT pathway.

23. The nucleic acid expression system of claim 22, wherein the inhibitor of a JAK-STAT pathway is or comprises a polypeptide agent that inhibits activity and/or formation of a complex comprising STAT1, STAT2, and IRF9, or a nucleic acid encoding the polypeptide agent.

24. The nucleic acid expression system of claim 23, wherein the polypeptide agent that inhibits activity and/or formation of the STAT1/STAT2/IRF9 complex is or comprises a polypeptide agent that binds to and/or inhibits activity and/or interaction of at least one of STAT1, STAT2, and IRF9.

25-27. (canceled)

28. A composition comprising the nucleic acid expression system of claim 1.

29-30. (canceled)

31. A cell comprising the nucleic acid expression system of claim 1.

32-33. (canceled)

34. A pharmaceutical composition comprising a composition that delivers an inhibitor of a NF-κB pathway and a composition that delivers an inhibitor of an IRF pathway.

35-54. (canceled)

55. A method comprising: contacting a target cell with at least one of:
(a) an RNA oligonucleotide comprising a payload sequence; and
(b) at least one composition that delivers an inhibitor of a NF-κB pathway and/or an inhibitor of an IRF pathway,
so that the target cell is receiving a nucleic acid expression system of claim 1.

56-77. (canceled)

78. A kit comprising:
a. a container including an RNA oligonucleotide comprising a payload sequence, and
b. at least one container including at least one composition that delivers an inhibitor of a NF-κB pathway and/or an inhibitor of an IRF pathway.

79.-82. (canceled)


SEQUENCE LISTING (12 sequences)

SEQ ID NO: 1 - DNA, 45 bp, Artificial Sequence
  Description of Artificial Sequence Synthetic primer
gaatttaatacgactcactatagggcttgttctttttgcagaagc

SEQ ID NO: 2 - DNA, 149 bp, Artificial Sequence
  Description of Artificial Sequence Synthetic primer
ttttttttttttttttttttttttttttttttttttttttttttttttttttttttttttttttttttttttttttttttttttttttttttttttttttttttttttttttttttttttagaatgtgaagaaactttctttttattag

SEQ ID NO: 3 - DNA, 634 bp, Artificial Sequence
  Description of Artificial Sequence Synthetic polynucleotide
cttgttctttttgcagaagctcagaataaacgctcaactttggccaccatgacggccaactttagtacccacgtcttttcaccacagcactgtggatgtgacagactgaccagtattgatgacgtcagacaatgtttgactgaatatatttattggtcgtcctatgcataccgcaacaggcaatgcgctggacaattgtattccacactcctctcttttagagatgatgcggaattagtgttcatcgacattcgcgagctggtaaaaaatatgccgtgggatgatgtcaaagattgtgcagaaatcatccgttgttatataccggatgagcaaaaaaccatcagagagatttcggccatcatcggactttgtgcatatgctgctacttactggggaggtgaagaccatcccactagtaacagtctgaacgcattgtttgtgatgcttgagatgctcaattacgtggattataacatcatattccggcgtatgaattaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 4 - DNA, 447 bp, Vaccinia virus
atgacggccaactttagtacccacgtcttttcaccacagcactgtggatgtgacagactgaccagtattgatgacgtcagacaatgtttgactgaatatatttattggtcgtcctatgcataccgcaacaggcaatgcgctggacaattgtattccacactcctctcttttagagatgatgcggaattagtgttcatcgacattcgcgagctggtaaaaaatatgccgtgggatgatgtcaaagattgtgcagaaatcatccgttgttatataccggatgagcaaaaaaccatcagagagatttcggccatcatcggactttgtgcatatgctgctacttactggggaggtgaagaccatcccactagtaacagtctgaacgcattgtttgtgatgcttgagatgctcaattacgtggattataacatcatattccggcgtatgaat

SEQ ID NO: 5 - protein, 149 aa, Vaccinia virus
MTANFSTHVFSPQHCGCDRLTSIDDVRQCLTEYIYWSSYAYRNRQCAGQLYSTLLSFRDDAELVFIDIRELVKNMPWDDVKDCAEIIRCYIPDEQKTIREISAIIGLCAYAATYWGGEDHPTSNSLNALFVMLEMLNYVDYNIIFRRMN

SEQ ID NO: 6 - DNA, 45 bp, Artificial Sequence
  Description of Artificial Sequence Synthetic primer
gaatttaatacgactcactataaggcttgttctttttgcagaagc

SEQ ID NO: 7 - DNA, 634 bp, Artificial Sequence
  Description of Artificial Sequence Synthetic polynucleotide
cttgttctttttgcagaagctcagaataaacgctcaactttggccaccatggcgactaaattagattatgaggatgctgttttttactttgtggatgatgataaaatatgtagtcgcgactccatcatcgatctaatagatgaatatattacgtggagaaatcatgttatagtgtttaacaaagatattaccagttgtggaagactgtacaaggaattgatgaagttcgatgatgtcgctatacggtactatggtattgataaaattaatgagattgtcgaagctatgagcgaaggagaccactacatcaattttacaaaagtccatgatcaggaaagtttattcgctaccataggaatatgtgctaaaatcactgaacattggggatacaaaaagatttcagaatctagattccaatcattgggaaacattacagatctgatgaccgacgataatataaacatcttgatactttttctagaaaaaaaattgaattaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 8 - DNA, 447 bp, Vaccinia virus
atggcgactaaattagattatgaggatgctgttttttactttgtggatgatgataaaatatgtagtcgcgactccatcatcgatctaatagatgaatatattacgtggagaaatcatgttatagtgtttaacaaagatattaccagttgtggaagactgtacaaggaattgatgaagttcgatgatgtcgctatacggtactatggtattgataaaattaatgagattgtcgaagctatgagcgaaggagaccactacatcaattttacaaaagtccatgatcaggaaagtttattcgctaccataggaatatgtgctaaaatcactgaacattggggatacaaaaagatttcagaatctagattccaatcattgggaaacattacagatctgatgaccgacgataatataaacatcttgatactttttctagaaaaaaaattgaat

SEQ ID NO: 9 - protein, 149 aa, Vaccinia virus
MATKLDYEDAVFYFVDDDKICSRDSIIDLIDEYITWRNHVIVFNKDITSCGRLYKELMKFDDVAIRYYGIDKINEIVEAMSEGDHYINFTKVHDQESLFATIGICAKITEHWGYKKISESRFQSLGNITDLMTDDNINILILFLEKKLN

SEQ ID NO: 10 - DNA, 640 bp, Artificial Sequence
  Description of Artificial Sequence Synthetic polynucleotide
cttgttctttttgcagaagctcagaataaacgctcaactttggccaccatgaatgcgtataataaagccgattcgttttctttagagtctgattctatcaaagatgttatacacgattatatttgttggctcagtatgactgatgaaatgagaccatctatcggaaacgtctttaaagcgatggaaacgtttaagatagacgcggttagatattacgatggtaacatatatgaattagctaaagatataaatgcgatgtcgtttgacggttttataagatctctacaaactatcgcttcaaagaaagataaactcactgtttatggaaccatgggactgctgtctattgtcgtagatattaacaaaggttgtgatatatccaatatcaagttcgctgccggaataatcattttaatggagtatatttttgatgacacggatatgtctcatcttaaagtagcactctatcgtagaatacagagacgtgatgatgtagatagataatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 11 - DNA, 453 bp, Vaccinia virus
atgaatgcgtataataaagccgattcgttttctttagagtctgattctatcaaagatgttatacacgattatatttgttggctcagtatgactgatgaaatgagaccatctatcggaaacgtctttaaagcgatggaaacgtttaagatagacgcggttagatattacgatggtaacatatatgaattagctaaagatataaatgcgatgtcgtttgacggttttataagatctctacaaactatcgcttcaaagaaagataaactcactgtttatggaaccatgggactgctgtctattgtcgtagatattaacaaaggttgtgatatatccaatatcaagttcgctgccggaataatcattttaatggagtatatttttgatgacacggatatgtctcatcttaaagtagcactctatcgtagaatacagagacgtgatgatgtagataga

SEQ ID NO: 12 - protein, 151 aa, Vaccinia virus
MNAYNKADSFSLESDSIKDVIHDYICWLSMTDEMRPSIGNVFKAMETFKIDAVRYYDGNIYELAKDINAMSFDGFIRSLQTIASKKDKLTVYGTMGLLSIVVDINKGCDISNIKFAAGIIILMEYIFDDTDMSHLKVALYRRIQRRDDVDR